SEQUENCE LISTING (257 sequences)

SEQ ID NO: 1 - DNA, 33 bp, Artificial Sequence
  Description of Artificial Sequence: Synthetic primer
aattgcggccgctggaagggctaatttggtccc

SEQ ID NO: 2 - DNA, 23 bp, Artificial Sequence
  Description of Artificial Sequence: Synthetic primer
tgtcacttccccttggttctctc

SEQ ID NO: 3 - DNA, 32 bp, Artificial Sequence
  Description of Artificial Sequence: Synthetic primer
aaaagaattcgtggatctaccacacacaaggc

SEQ ID NO: 4 - DNA, 30 bp, Artificial Sequence
  Description of Artificial Sequence: Synthetic primer
aaaaggatccaccatttgcccctggaggtt

SEQ ID NO: 5 - DNA, 27 bp, Artificial Sequence
  Description of Artificial Sequence: Synthetic primer
gaaagcgaaagtaaagccagaggagat

SEQ ID NO: 6 - DNA, 26 bp, Artificial Sequence
  Description of Artificial Sequence: Synthetic primer
acacaacagacgggcacacactactt

SEQ ID NO: 7 - DNA, 37 bp, Artificial Sequence
  Description of Artificial Sequence: Synthetic primer
aaaagaattcgacagctacaaccatcccttcagacag

SEQ ID NO: 8 - DNA, 35 bp, Artificial Sequence
  Description of Artificial Sequence: Synthetic primer
aaaaggatccagcagtgggttccctagttagccag

SEQ ID NO: 9 - DNA, 22 bp, Artificial Sequence
  Description of Artificial Sequence: Synthetic primer
ttggcagaactacacaccaggg

SEQ ID NO: 10 - DNA, 24 bp, Artificial Sequence
  Description of Artificial Sequence: Synthetic primer
ccgagagctcccaggctcagatct

SEQ ID NO: 11 - DNA, 24 bp, Artificial Sequence
  Description of Artificial Sequence: Synthetic primer
ttagcagaactacacaccagggcc

SEQ ID NO: 12 - DNA, 23 bp, Artificial Sequence
  Description of Artificial Sequence: Synthetic primer
gctgcttatatgtagcatctgag

SEQ ID NO: 13 - DNA, 20 bp, Artificial Sequence
  Description of Artificial Sequence: Synthetic primer
cccttggacccagaggttct

SEQ ID NO: 14 - DNA, 20 bp, Artificial Sequence
  Description of Artificial Sequence: Synthetic primer
cgagcactttcttgccatga

SEQ ID NO: 15 - DNA, 20 bp, Artificial Sequence
  Description of Artificial Sequence: Synthetic primer
cccttggacccagcggtact

SEQ ID NO: 16 - DNA, 20 bp, Artificial Sequence
  Description of Artificial Sequence: Synthetic primer
gttatcaccttcttgccatg

SEQ ID NO: 17 - DNA, 20 bp, Artificial Sequence
  Description of Artificial Sequence: Synthetic primer
tccagcagagacagggcaag

SEQ ID NO: 18 - DNA, 22 bp, Artificial Sequence
  Description of Artificial Sequence: Synthetic primer
tgccaaattcctgcttgatccc

SEQ ID NO: 19 - DNA, 26 bp, Artificial Sequence
  Description of Artificial Sequence: Synthetic probe
cgcccaccaacaggcggccttaactg

SEQ ID NO: 20 - DNA, 21 bp, Artificial Sequence
  Description of Artificial Sequence: Synthetic primer
tccttgggatgttgatgatct

SEQ ID NO: 21 - DNA, 20 bp, Artificial Sequence
  Description of Artificial Sequence: Synthetic primer
tggcccaaacattatgtacc

SEQ ID NO: 22 - DNA, 23 bp, Artificial Sequence
  Description of Artificial Sequence: Synthetic probe
tggtggttgcttctttccacaca

SEQ ID NO: 23 - DNA, 20 bp, Artificial Sequence
  Description of Artificial Sequence: Synthetic primer
cccttggacccagaggttct

SEQ ID NO: 24 - DNA, 20 bp, Artificial Sequence
  Description of Artificial Sequence: Synthetic primer
cgagcactttcttgccatga

SEQ ID NO: 25 - DNA, 41 bp, Artificial Sequence
  Description of Artificial Sequence: Synthetic probe
gcgagcatctgtccactcctgatgctgttatgggcgctcgc

SEQ ID NO: 26 - DNA, 21 bp, Artificial Sequence
  Description of Artificial Sequence: Synthetic primer
gcagaactacacaccagggcc

SEQ ID NO: 27 - DNA, 21 bp, Artificial Sequence
  Description of Artificial Sequence: Synthetic primer
ggatagatgtaaaagacacca

SEQ ID NO: 28 - DNA, 20 bp, Artificial Sequence
  Description of Artificial Sequence: Synthetic primer
cgccaacaagttgacgagat

SEQ ID NO: 29 - DNA, 20 bp, Artificial Sequence
  Description of Artificial Sequence: Synthetic primer
tcgactacaacctgctgacc

SEQ ID NO: 30 - DNA, 18 bp, Artificial Sequence
  Description of Artificial Sequence: Synthetic primer
ggtgggcttcttctgctt

SEQ ID NO: 31 - DNA, 21 bp, Artificial Sequence
  Description of Artificial Sequence: Synthetic primer
ctacaatgagctgcgtgtggc

SEQ ID NO: 32 - DNA, 21 bp, Artificial Sequence
  Description of Artificial Sequence: Synthetic primer
caggtccagacgcaggatggc

SEQ ID NO: 33 - skipped

SEQ ID NO: 34 - DNA, 20 bp, Artificial Sequence
  Description of Artificial Sequence: Synthetic primer
gagtgaccttcccaaattgc

SEQ ID NO: 35 - DNA, 20 bp, Artificial Sequence
  Description of Artificial Sequence: Synthetic primer
atggtgaggtgagggatgag

SEQ ID NO: 36 - DNA, 20 bp, Artificial Sequence
  Description of Artificial Sequence: Synthetic primer
cagactctgatgggtggcag

SEQ ID NO: 37 - DNA, 20 bp, Artificial Sequence
  Description of Artificial Sequence: Synthetic primer
gctaaggagagagggtggga

SEQ ID NO: 38 - DNA, 21 bp, Artificial Sequence
  Description of Artificial Sequence: Synthetic primer
ccaagtggccctcagattaca

SEQ ID NO: 39 - DNA, 22 bp, Artificial Sequence
  Description of Artificial Sequence: Synthetic primer
tcattcaccccaaatcctacgg

SEQ ID NO: 40 - DNA, 20 bp, Artificial Sequence
  Description of Artificial Sequence: Synthetic primer
cattaaccacctggggaaca

SEQ ID NO: 41 - DNA, 20 bp, Artificial Sequence
  Description of Artificial Sequence: Synthetic primer
tctcagacccaggaatgtca

SEQ ID NO: 42 - DNA, 20 bp, Artificial Sequence
  Description of Artificial Sequence: Synthetic primer
gaggactctccagccaaagg

SEQ ID NO: 43 - DNA, 20 bp, Artificial Sequence
  Description of Artificial Sequence: Synthetic primer
gagctgggggtcttagagga

SEQ ID NO: 44 - DNA, 21 bp, Artificial Sequence
  Description of Artificial Sequence: Synthetic primer
tgcaccagccaaaacttagga

SEQ ID NO: 45 - DNA, 22 bp, Artificial Sequence
  Description of Artificial Sequence: Synthetic primer
tctaattaggtggcagcacgtt

SEQ ID NO: 46 - DNA, 24 bp, Artificial Sequence
  Description of Artificial Sequence: Synthetic primer
tcagcccagaagtaatacccatgt

SEQ ID NO: 47 - DNA, 22 bp, Artificial Sequence
  Description of Artificial Sequence: Synthetic primer
cactgtgtttagcatggtgttt

SEQ ID NO: 48 - DNA, 27 bp, Human immunodeficiency virus 1
gcagaactacacaccagggccagggat

SEQ ID NO: 49 - DNA, 26 bp, Homo sapiens
tctaaactccacaccagggccatgaa

SEQ ID NO: 50 - DNA, 26 bp, Homo sapiens
tcagatctccacaccagagccacgag

SEQ ID NO: 51 - DNA, 26 bp, Homo sapiens
acaggccaacccaccagggcccagag

SEQ ID NO: 52 - DNA, 26 bp, Homo sapiens
gtaggactacgcaccagggcaaagag

SEQ ID NO: 53 - DNA, 26 bp, Homo sapiens
acaaaagtacacaccagagcctgggg

SEQ ID NO: 54 - DNA, 26 bp, Homo sapiens
tgtgaactacgccccagggcctggaa

SEQ ID NO: 55 - DNA, 26 bp, Homo sapiens
acagagctgagcaccagggcccaggg

SEQ ID NO: 56 - DNA, 26 bp, Homo sapiens
ccagttctccaccccagggccatgga

SEQ ID NO: 57 - DNA, 26 bp, Homo sapiens
ccagagctgcttaccagggccatgga

SEQ ID NO: 58 - DNA, 26 bp, Homo sapiens
acagcactccccaccagggcttgggg

SEQ ID NO: 59 - DNA, 26 bp, Homo sapiens
acagaacgtcacaccagggtcaggag

SEQ ID NO: 60 - DNA, 26 bp, Homo sapiens
acaaaactagacagcagggccaggag

SEQ ID NO: 61 - DNA, 26 bp, Homo sapiens
tgagcacttcacagcagggccgggaa

SEQ ID NO: 62 - DNA, 26 bp, Homo sapiens
gcagcactacacatcagggctaagaa

SEQ ID NO: 63 - DNA, 26 bp, Homo sapiens
ccgcaactccacagcagggccaggga

SEQ ID NO: 64 - DNA, 26 bp, Homo sapiens
ctagaggaacacaccagggcctggga

SEQ ID NO: 65 - DNA, 26 bp, Homo sapiens
acagccccagacaccagggcctggag

SEQ ID NO: 66 - DNA, 26 bp, Homo sapiens
ccaggtctacccagcagggccaggag

SEQ ID NO: 67 - DNA, 26 bp, Homo sapiens
acaggagggcacaccagggcccagga

SEQ ID NO: 68 - DNA, 26 bp, Homo sapiens
acagaaataaacaccagggcttcggg

SEQ ID NO: 69 - DNA, 26 bp, Homo sapiens
gcagaactgcagaccaggggctgggg

SEQ ID NO: 70 - DNA, 26 bp, Homo sapiens
ccagagcaccaaaccagggcccagga

SEQ ID NO: 71 - DNA, 26 bp, Homo sapiens
gcagagctccccaccaggggcaggga

SEQ ID NO: 72 - DNA, 26 bp, Homo sapiens
acaggcccacactccagggcccagaa

SEQ ID NO: 73 - DNA, 26 bp, Homo sapiens
gcagtgccacactccagggccttggg

SEQ ID NO: 74 - DNA, 26 bp, Homo sapiens
gcaggagtaggcaccagggccctgag

SEQ ID NO: 75 - DNA, 26 bp, Homo sapiens
gcagcaccacacaccaggcccaggag

SEQ ID NO: 76 - DNA, 26 bp, Homo sapiens
gcagagctagccaccagggcttagga

SEQ ID NO: 77 - DNA, 26 bp, Homo sapiens
gcagagctccagcccagggcctgggg

SEQ ID NO: 78 - DNA, 26 bp, Homo sapiens
ggggaaatacacatcagggccaggaa

SEQ ID NO: 79 - DNA, 26 bp, Homo sapiens
agagaatttcacaacagggccctgaa

SEQ ID NO: 80 - DNA, 26 bp, Homo sapiens
acaaacctacagaccagagcccaggg

SEQ ID NO: 81 - DNA, 26 bp, Homo sapiens
catgagctacacaccaggaccaggag

SEQ ID NO: 82 - DNA, 26 bp, Homo sapiens
gaaaaactacagaccagggacaaggg

SEQ ID NO: 83 - DNA, 26 bp, Homo sapiens
ccagaactcagccccagggccctggg

SEQ ID NO: 84 - DNA, 26 bp, Homo sapiens
gctggcctacacaccaggcccagggg

SEQ ID NO: 85 - DNA, 26 bp, Homo sapiens
cctgaaccacaccccagggctcaggg

SEQ ID NO: 86 - DNA, 26 bp, Homo sapiens
gcagaacaccaagccagggccaggaa

SEQ ID NO: 87 - DNA, 26 bp, Homo sapiens
gaatagctacacactagggccatgga

SEQ ID NO: 88 - DNA, 26 bp, Homo sapiens
gaagaaccacaaaacagggcccagaa

SEQ ID NO: 89 - DNA, 26 bp, Homo sapiens
atagtactacactcctgggcctcgag

SEQ ID NO: 90 - DNA, 26 bp, Homo sapiens
gaagaacaacacagcagggcagagag

SEQ ID NO: 91 - DNA, 26 bp, Homo sapiens
ccagaaacacccaccagtgcccggga

SEQ ID NO: 92 - DNA, 26 bp, Homo sapiens
ccagagctgcagacccgggccccggg

SEQ ID NO: 93 - DNA, 26 bp, Homo sapiens
ccagaccgagacaccaggggcggggg

SEQ ID NO: 94 - DNA, 26 bp, Homo sapiens
ccagatctagactccagggcagtgag

SEQ ID NO: 95 - DNA, 26 bp, Homo sapiens
tcagagctagactccagggctggggg

SEQ ID NO: 96 - DNA, 26 bp, Homo sapiens
ggagaacttaacaccaggtccctggg

SEQ ID NO: 97 - DNA, 26 bp, Homo sapiens
ccagcaccacagagcagggcctggga

SEQ ID NO: 98 - DNA, 27 bp, Human immunodeficiency virus 1
ggatagatgtaaaagacaccaaggaag

SEQ ID NO: 99 - DNA, 26 bp, Homo sapiens
agaaaaatgtaaaagacaccttggaa

SEQ ID NO: 100 - DNA, 26 bp, Homo sapiens
ttatacatttgaaagacaccaaagaa

SEQ ID NO: 101 - DNA, 26 bp, Homo sapiens
ggataaatgggaaagacaccagggga

SEQ ID NO: 102 - DNA, 26 bp, Homo sapiens
tcttagacttaaaagacaccattgaa

SEQ ID NO: 103 - DNA, 26 bp, Homo sapiens
acattgaattaaaagacaccatagag

SEQ ID NO: 104 - DNA, 26 bp, Homo sapiens
ggatagagccaaaagacaccaaagag

SEQ ID NO: 105 - DNA, 26 bp, Homo sapiens
aaatagctcttaaagacaccagcgaa

SEQ ID NO: 106 - DNA, 26 bp, Homo sapiens
agatcaatgtaaaagtcaccatcgaa

SEQ ID NO: 107 - DNA, 26 bp, Homo sapiens
ttttagatgtaaaagacatcagggag

SEQ ID NO: 108 - DNA, 26 bp, Homo sapiens
tgataaatgaaacagacaccagagga

SEQ ID NO: 109 - DNA, 26 bp, Homo sapiens
gaaaagatttaagagacaccaaagag

SEQ ID NO: 110 - DNA, 26 bp, Homo sapiens
agggagatctaagagacaccagagag

SEQ ID NO: 111 - DNA, 26 bp, Homo sapiens
atgcagatgtaacagacaccagggaa

SEQ ID NO: 112 - DNA, 26 bp, Homo sapiens
gtatggatgttaaagactccattgag

SEQ ID NO: 113 - DNA, 26 bp, Homo sapiens
cggtagattttaaagactccaaagag

SEQ ID NO: 114 - DNA, 26 bp, Homo sapiens
agagagatattaaagaccccagtgaa

SEQ ID NO: 115 - DNA, 26 bp, Homo sapiens
ggataaatgtgaaagacatcatagaa

SEQ ID NO: 116 - DNA, 26 bp, Homo sapiens
agaaggaggaaaaagacaccagggag

SEQ ID NO: 117 - DNA, 26 bp, Homo sapiens
taataggtagaaaagacaccagtgaa

SEQ ID NO: 118 - DNA, 26 bp, Homo sapiens
ccaaagatgaaaaagacacccgagaa

SEQ ID NO: 119 - DNA, 26 bp, Homo sapiens
ttataaatgcaaaagacacccatgaa

SEQ ID NO: 120 - DNA, 26 bp, Homo sapiens
ggctgggtgaaaaagacaccatggaa

SEQ ID NO: 121 - DNA, 26 bp, Homo sapiens
ggacagatgtgaaagagaccaaagga

SEQ ID NO: 122 - DNA, 26 bp, Homo sapiens
tgatgcaagtaacagacaccatggga

SEQ ID NO: 123 - DNA, 26 bp, Homo sapiens
caatagttgttcaagacaccagtgaa

SEQ ID NO: 124 - DNA, 26 bp, Homo sapiens
agaaagatacagaagacaccagggag

SEQ ID NO: 125 - DNA, 26 bp, Homo sapiens
tgagacttgtacaagacaccacgggg

SEQ ID NO: 126 - DNA, 26 bp, Homo sapiens
agattgttggtaaagacaccacagag

SEQ ID NO: 127 - DNA, 26 bp, Homo sapiens
ggaaagttataaaagacaccggggaa

SEQ ID NO: 128 - DNA, 26 bp, Homo sapiens
ccattgatctaaaagtcaccactgga

SEQ ID NO: 129 - DNA, 26 bp, Homo sapiens
aaatacctgtaagagacaccactgag

SEQ ID NO: 130 - DNA, 26 bp, Homo sapiens
tggtagatttataagacaccgtaggg

SEQ ID NO: 131 - DNA, 26 bp, Homo sapiens
gaatggatgtgaaaggcaccactgaa

SEQ ID NO: 132 - DNA, 26 bp, Homo sapiens
aaataaatgtgaaagtcaccacagaa

SEQ ID NO: 133 - DNA, 26 bp, Homo sapiens
agatggatggcatagacaccacgggg

SEQ ID NO: 134 - DNA, 26 bp, Homo sapiens
tgaaagatcttaaagccaccaaagga

SEQ ID NO: 135 - DNA, 26 bp, Homo sapiens
gagtagatctaaaagacagcaaggaa

SEQ ID NO: 136 - DNA, 26 bp, Homo sapiens
tcatatgtgtaaaagacacaaaggag

SEQ ID NO: 137 - DNA, 26 bp, Homo sapiens
ggttagcgggaaaagacaccacaggg

SEQ ID NO: 138 - DNA, 26 bp, Homo sapiens
ggattcatgcaaaagacactataggg

SEQ ID NO: 139 - DNA, 26 bp, Homo sapiens
agaaatatctaaaagacaacaaagag

SEQ ID NO: 140 - DNA, 26 bp, Homo sapiens
ggaaaggagcaaaagacaccagaggg

SEQ ID NO: 141 - DNA, 26 bp, Homo sapiens
agattcatttaaaagacaacaaagaa

SEQ ID NO: 142 - DNA, 26 bp, Homo sapiens
agagatatgtataagacacaatagga

SEQ ID NO: 143 - DNA, 26 bp, Homo sapiens
agatagaaatgaaagacactagtgaa

SEQ ID NO: 144 - DNA, 26 bp, Homo sapiens
tgataaatgggaatgacaccagagag

SEQ ID NO: 145 - DNA, 27 bp, Homo sapiens
acagcactccccaccagggcttggggg

SEQ ID NO: 146 - DNA, 27 bp, Homo sapiens
acaaacctacagaccagagcccagggt

SEQ ID NO: 147 - DNA, 27 bp, Homo sapiens
ccagaccgagacaccaggggcggggga

SEQ ID NO: 148 - DNA, 27 bp, Homo sapiens
ccaaagatgaaaaagacacccgagaaa

SEQ ID NO: 149 - DNA, 27 bp, Homo sapiens
tgagacttgtacaagacaccacggggc

SEQ ID NO: 150 - DNA, 27 bp, Homo sapiens
agaaaaatgtaaaagacaccttggaaa

SEQ ID NO: 151 - DNA, 29 bp, Artificial Sequence
  Description of Artificial Sequence: Synthetic oligonucleotide
gcagactacacaccaggccaaggaagcct

SEQ ID NO: 152 - DNA, 31 bp, Artificial Sequence
  Description of Artificial Sequence: Synthetic oligonucleotide
aaaggatagatgtaaaagacagccaggggtc

SEQ ID NO: 153 - DNA, 30 bp, Artificial Sequence
  Description of Artificial Sequence: Synthetic oligonucleotide
ggcagaactacacaccagggccaggggtca

SEQ ID NO: 154 - DNA, 41 bp, Artificial Sequence
  Description of Artificial Sequence: Synthetic oligonucleotide
aaagtagcaggaccacttctgcgctcggcccttccggctgg

SEQ ID NO: 155 - DNA, 160 bp, Artificial Sequence
  Description of Artificial Sequence: Synthetic polynucleotide
gttacatcgaactggatctcaacagcggtaagatccttgatgtaacccactcgtgcacccaactgatcttcagcatctggtgctgtgctgacaactggtagttcctgccctacatcttccccagaagcgtgtccttcgacaacagcttcaacaacaaggt

SEQ ID NO: 156 - DNA, 31 bp, Artificial Sequence
  Description of Artificial Sequence: Synthetic oligonucleotide
ggcagaactacacaccaggccaaggaagcct

SEQ ID NO: 157 - DNA, 54 bp, Artificial Sequence
  Description of Artificial Sequence: Synthetic oligonucleotide
ttggcagaactacacaccagggccagggatcagatatccactgacctttggatg

SEQ ID NO: 158 - DNA, 29 bp, Artificial Sequence
  Description of Artificial Sequence: Synthetic oligonucleotide
catccggagtactacaaagactgctgaca

SEQ ID NO: 159 - DNA, 26 bp, Artificial Sequence
  Description of Artificial Sequence: Synthetic oligonucleotide
gccctcagatgctacatataagcagc

SEQ ID NO: 160 - DNA, 45 bp, Artificial Sequence
  Description of Artificial Sequence: Synthetic oligonucleotide
ttagcagaactacacaccagggccaggggtcagatatccactgac

SEQ ID NO: 161 - DNA, 20 bp, Artificial Sequence
  Description of Artificial Sequence: Synthetic oligonucleotide
tacttcaagaactgctgaca

SEQ ID NO: 162 - DNA, 40 bp, Artificial Sequence
  Description of Artificial Sequence: Synthetic oligonucleotide
ggggtcagatatccactgactacttcaagaactgctgaca

SEQ ID NO: 163 - DNA, 54 bp, Artificial Sequence
  Description of Artificial Sequence: Synthetic oligonucleotide
ttagcagaactacacaccagggccagggatcagatatccactgacctttggatg

SEQ ID NO: 164 - DNA, 29 bp, Artificial Sequence
  Description of Artificial Sequence: Synthetic oligonucleotide
catccggagtacttcaagaactgctgaca

SEQ ID NO: 165 - DNA, 26 bp, Artificial Sequence
  Description of Artificial Sequence: Synthetic oligonucleotide
gccctcagatcctgcatataagcagc

SEQ ID NO: 166 - DNA, 45 bp, Artificial Sequence
  Description of Artificial Sequence: Synthetic oligonucleotide
ttagcagaactacacaccagggccagggatcagatatccactgac

SEQ ID NO: 167 - DNA, 40 bp, Artificial Sequence
  Description of Artificial Sequence: Synthetic oligonucleotide
ttagcagaactacacaccagggccagggatcagatatcca

SEQ ID NO: 168 - DNA, 40 bp, Artificial Sequence
  Description of Artificial Sequence: Synthetic oligonucleotide
gggatcagatatccactgactacttcaagaactgctgaca

SEQ ID NO: 169 - DNA, 15 bp, Artificial Sequence
  Description of Artificial Sequence: Synthetic oligonucleotide
gggatcagatatcca

SEQ ID NO: 170 - DNA, 33 bp, Artificial Sequence
  Description of Artificial Sequence: Synthetic oligonucleotide
aggcttccttggcctggtgtgtagttctgccaa

SEQ ID NO: 171 - DNA, 39 bp, Artificial Sequence
  Description of Artificial Sequence: Synthetic oligonucleotide
aaaggatagatgtaaaagacagccaggggtcagatatcc

SEQ ID NO: 172 - DNA, 74 bp, Artificial Sequence
  Description of Artificial Sequence: Synthetic oligonucleotide
ttggcagaactacacaccaggaaagttgcaggaccacttctgcgctcggcccttccggctggccaaggaagcct

SEQ ID NO: 173 - DNA, 202 bp, Artificial Sequence
  Description of Artificial Sequence: Synthetic polynucleotide
aggcttccttggaccttgttgttgaagctgttgtcgaaggacacgcttctggggaagatgtagggcaggaactaccagttgtcatgtcagcacagcaccagatgctgaagatcagttgggtgcacgagtgggttacatcaaggatcttaccgctgttgagatccagttcagttcgatgtaaccctggtgtgtagttctgcca

SEQ ID NO: 174 - DNA, 33 bp, Artificial Sequence
  Description of Artificial Sequence: Synthetic oligonucleotide
aaaggatagatgtaaaagacagccaggggtcag

SEQ ID NO: 175 - DNA, 33 bp, Artificial Sequence
  Description of Artificial Sequence: Synthetic oligonucleotide
  modified_base (33)..(33): a, c, t, g, unknown or other
aaaggatagatgtaaaagacagccaggggtcan

SEQ ID NO: 176 - DNA, 33 bp, Artificial Sequence
  Description of Artificial Sequence: Synthetic oligonucleotide
aaaggatagatgtaaaagacagccagggatcag

SEQ ID NO: 177 - DNA, 27 bp, Artificial Sequence
  Description of Artificial Sequence: Synthetic oligonucleotide
aaaggatagatgtaaaagacaatatcc

SEQ ID NO: 178 - DNA, 28 bp, Artificial Sequence
  Description of Artificial Sequence: Synthetic oligonucleotide
aaaggatagatgtaaaagacacaaggag

SEQ ID NO: 179 - DNA, 28 bp, Artificial Sequence
  Description of Artificial Sequence: Synthetic oligonucleotide
aggcttccttggtgtgtagttctgccaa

SEQ ID NO: 180 - DNA, 116 bp, Artificial Sequence
  Description of Artificial Sequence: Synthetic polynucleotide
tggatctaccacacacaaggctacttccctgattggcagaactacacaccagggccagggatcagatatccactgacctttggatggtgcttcaagttagtaccagttgaaccaga

SEQ ID NO: 181 - DNA, 160 bp, Artificial Sequence
  Description of Artificial Sequence: Synthetic polynucleotide
aaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggt

SEQ ID NO: 182 - DNA, 105 bp, Artificial Sequence
  Description of Artificial Sequence: Synthetic polynucleotide
ccacacacaaggctacttccctgattggcagaactacacaccagggccaggggtcagatatccactgacctttggatggtgctacaagctagtaccagttgagcc

SEQ ID NO: 183 - DNA, 126 bp, Artificial Sequence
  Description of Artificial Sequence: Synthetic polynucleotide
agataaggtagaggaagagcaaaacaaaagtaagaaaaaggcacagcaagcagcagctgacacaggaaacaacagccaggtcagccaaaattaccctatagtgcagaacctccaggggcaaatggt

SEQ ID NO: 184 - DNA, 110 bp, Artificial Sequence
  Description of Artificial Sequence: Synthetic polynucleotide
  modified_base (4)..(5): a, c, t, g, unknown or other
aggnnccacacacaaggctacttccctgattggcagaactacacaccagggccaggggtcagatatccactgacctttggatggtgctacaagctagtaccagttgagcc

SEQ ID NO: 185 - DNA, 118 bp, Artificial Sequence
  Description of Artificial Sequence: Synthetic polynucleotide
  modified_base (80)..(80): a, c, t, g, unknown or other
  modified_base (85)..(85): a, c, t, g, unknown or other
agataaggtagaggaggagcaaaacaaaagtaagaaaaaggcacagcaagcagcagctgacacaggaaacaacagccagntcagncaatatagtgcagaacatccaggggcaaatggt

SEQ ID NO: 186 - DNA, 33 bp, Artificial Sequence
  Description of Artificial Sequence: Synthetic oligonucleotide
ttggcagaactacacaccagggccaggggtcag

SEQ ID NO: 187 - DNA, 404 bp, Artificial Sequence
  Description of Artificial Sequence: Synthetic polynucleotide
gatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 188 - DNA, 546 bp, Artificial Sequence
  Description of Artificial Sequence: Synthetic polynucleotide
  modified_base (1)..(6): a, c, t, g, unknown or other
  modified_base (9)..(9): a, c, t, g, unknown or other
  modified_base (11)..(11): a, c, t, g, unknown or other
  modified_base (62)..(62): a, c, t, g, unknown or other
  modified_base (65)..(67): a, c, t, g, unknown or other
  modified_base (70)..(70): a, c, t, g, unknown or other
  modified_base (74)..(74): a, c, t, g, unknown or other
  modified_base (133)..(133): a, c, t, g, unknown or other
  modified_base (224)..(224): a, c, t, g, unknown or other
  modified_base (248)..(248): a, c, t, g, unknown or other
  modified_base (253)..(253): a, c, t, g, unknown or other
  modified_base (256)..(256): a, c, t, g, unknown or other
  modified_base (261)..(262): a, c, t, g, unknown or other
  modified_base (274)..(274): a, c, t, g, unknown or other
  modified_base (286)..(286): a, c, t, g, unknown or other
  modified_base (294)..(298): a, c, t, g, unknown or other
  modified_base (303)..(303): a, c, t, g, unknown or other
  modified_base (321)..(321): a, c, t, g, unknown or other
  modified_base (337)..(337): a, c, t, g, unknown or other
  modified_base (358)..(358): a, c, t, g, unknown or other
  modified_base (360)..(360): a, c, t, g, unknown or other
  modified_base (365)..(365): a, c, t, g, unknown or other
  modified_base (383)..(383): a, c, t, g, unknown or other
  modified_base (385)..(385): a, c, t, g, unknown or other
  modified_base (396)..(396): a, c, t, g, unknown or other
  modified_base (401)..(401): a, c, t, g, unknown or other
  modified_base (418)..(418): a, c, t, g, unknown or other
  modified_base (421)..(421): a, c, t, g, unknown or other
  modified_base (446)..(446): a, c, t, g, unknown or other
  modified_base (451)..(451): a, c, t, g, unknown or other
  modified_base (455)..(455): a, c, t, g, unknown or other
  modified_base (466)..(466): a, c, t, g, unknown or other
  modified_base (470)..(470): a, c, t, g, unknown or other
  modified_base (472)..(472): a, c, t, g, unknown or other
  modified_base (489)..(489): a, c, t, g, unknown or other
  modified_base (495)..(495): a, c, t, g, unknown or other
  modified_base (501)..(501): a, c, t, g, unknown or other
  modified_base (503)..(506): a, c, t, g, unknown or other
  modified_base (508)..(508): a, c, t, g, unknown or other
  modified_base (518)..(518): a, c, t, g, unknown or other
  modified_base (525)..(525): a, c, t, g, unknown or other
  modified_base (529)..(529): a, c, t, g, unknown or other
  modified_base (534)..(534): a, c, t, g, unknown or other
  modified_base (540)..(546): a, c, t, g, unknown or other
nnnnnncananacttagatcattatataatacaatagcagtcctctattgtgtgcacctggnccnnntgntgcntccggattacttcaagaactgctgacatctagcttgctacaagggactttccgctgggnactttccagggaggcgtggcctgggcggtactggctagtgccgatccctccaaagctggaaaaaaacgcctgctttttacatgacctgggnctcttacaccctgccagatttganggaanggnctctnnggctaactaagnaacccactgctnaattcttnnnnnctacntttcatccgtggccccancgctgcatcctgagtnggtgaaaaactgctgacatcnancttgntacaagggattttccacngnggggtttcccnggggnggtgtgccgggggcganagnggggagtggggacccctcatatgcnacaanaaanagccgcttttngcgngnaagggctctctctttancaccanatctgntnnnngnctctctggtngtggggncccntgctnatttcnnnnnnn

SEQ ID NO: 189 - DNA, 30 bp, Artificial Sequence
  Description of Artificial Sequence: Synthetic oligonucleotide
gacagctacaaccatcccttcagacaggat

SEQ ID NO: 190 - DNA, 33 bp, Artificial Sequence
  Description of Artificial Sequence: Synthetic oligonucleotide
aaaggatagatgtaaaagacaccaaggaagcct

SEQ ID NO: 191 - DNA, 249 bp, Artificial Sequence
  Description of Artificial Sequence: Synthetic polynucleotide
aagagaccatcaatgaggaagctgcagaatgggatagattgcatccagtgcatgcagggcctattgcaccaggccagatgagagaaccaaggggaagtgacatagcaggaactactagtaaagaccaatgacttacaaggcagctgtagatcttagccactttttaaaagaaaaggggggactggaagggctaattcactcccaaagaagacaagatatccttgatctgtggatctaccacacacaagg

SEQ ID NO: 192 - DNA, 28 bp, Artificial Sequence
  Description of Artificial Sequence: Synthetic oligonucleotide
tctctggctaactagggaacccactgct

SEQ ID NO: 193 - DNA, 72 bp, Artificial Sequence
  Description of Artificial Sequence: Synthetic oligonucleotide
  modified_base (53)..(53): a, c, t, g, unknown or other
  modified_base (56)..(56): a, c, t, g, unknown or other
aagagaccatctctctggttagaccaaatctgagcctgggagctctctggctnctngggaacccgctgctgg

SEQ ID NO: 194 - DNA, 109 bp, Artificial Sequence
  Description of Artificial Sequence: Synthetic polynucleotide
  modified_base (1)..(5): a, c, t, g, unknown or other
  modified_base (7)..(10): a, c, t, g, unknown or other
  modified_base (12)..(16): a, c, t, g, unknown or other
  modified_base (18)..(26): a, c, t, g, unknown or other
  modified_base (29)..(30): a, c, t, g, unknown or other
  modified_base (32)..(33): a, c, t, g, unknown or other
  modified_base (35)..(37): a, c, t, g, unknown or other
  modified_base (40)..(40): a, c, t, g, unknown or other
  modified_base (42)..(42): a, c, t, g, unknown or other
  modified_base (50)..(50): a, c, t, g, unknown or other
  modified_base (53)..(54): a, c, t, g, unknown or other
  modified_base (61)..(61): a, c, t, g, unknown or other
  modified_base (63)..(63): a, c, t, g, unknown or other
nnnnnannnnannnnncnnnnnnnnnatnnannannnatntnggaagggntanntcactcncnaagcaagacaagatatccttgatctgtggatctaccacacacaagg

SEQ ID NO: 195 - DNA, 23 bp, Artificial Sequence
  Description of Artificial Sequence: Synthetic oligonucleotide
  modified_base (5)..(5): a, c, t, g, unknown or other
  modified_base (12)..(12): a, c, t, g, unknown or other
  modified_base (19)..(21): a, c, t, g, unknown or other
tctcnggctaanggggggnnnac

SEQ ID NO: 196 - DNA, 518 bp, Artificial Sequence
  Description of Artificial Sequence: Synthetic polynucleotide
tggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgct

SEQ ID NO: 197 - DNA, 480 bp, Artificial Sequence
  Description of Artificial Sequence: Synthetic polynucleotide
  modified_base (1)..(34): a, c, t, g, unknown or other
  modified_base (38)..(45): a, c, t, g, unknown or other
  modified_base (47)..(47): a, c, t, g, unknown or other
  modified_base (49)..(57): a, c, t, g, unknown or other
  modified_base (89)..(89): a, c, t, g, unknown or other
  modified_base (91)..(91): a, c, t, g, unknown or other
  modified_base (215)..(215): a, c, t, g, unknown or other
  modified_base (283)..(283): a, c, t, g, unknown or other
  modified_base (312)..(312): a, c, t, g, unknown or other
  modified_base (317)..(317): a, c, t, g, unknown or other
  modified_base (322)..(322): a, c, t, g, unknown or other
  modified_base (326)..(326): a, c, t, g, unknown or other
  modified_base (345)..(346): a, c, t, g, unknown or other
  modified_base (351)..(351): a, c, t, g, unknown or other
  modified_base (362)..(362): a, c, t, g, unknown or other
  modified_base (387)..(387): a, c, t, g, unknown or other
  modified_base (409)..(409): a, c, t, g, unknown or other
  modified_base (417)..(417): a, c, t, g, unknown or other
  modified_base (419)..(419): a, c, t, g, unknown or other
  modified_base (421)..(423): a, c, t, g, unknown or other
  modified_base (425)..(425): a, c, t, g, unknown or other
  modified_base (427)..(427): a, c, t, g, unknown or other
  modified_base (442)..(442): a, c, t, g, unknown or other
  modified_base (449)..(449): a, c, t, g, unknown or other
  modified_base (451)..(451): a, c, t, g, unknown or other
  modified_base (454)..(454): a, c, t, g, unknown or other
  modified_base (457)..(457): a, c, t, g, unknown or other
  modified_base (469)..(469): a, c, t, g, unknown or other
  modified_base (474)..(474): a, c, t, g, unknown or other
nnnnnnnnnnnnnnnnnnnnnnnnnnnnnnnnnnaacnnnnnnnngngnnnnnnnnntcccaaagaacacaatatatccttgatctaananatctaccacacacaaggctacttccctgattggcagaactacacaccagggccaggggtcagatacccactgatctttggatggtgctacaagctagtaccagttgagccagataaggtagaanaggccaataaaggaaagaacaccagcttgttacaccctgtgagcctgcatggaatggatgaccctganagagaaatgttagagtggaggtttgacanccccntagcntttnttcacgtggccccagagcnncttcnggagtacttcncaagaagcgcagacctagcttgttngggtggagaatccccggggggnttttcagngnannntncngggagggggactggngagtgananccncanatgctgatatanctctntttttg

SEQ ID NO: 198 - DNA, 13 bp, Artificial Sequence
  Description of Artificial Sequence: Synthetic oligonucleotide
tgcaaatgttaaa

SEQ ID NO: 199 - DNA, 13 bp, Artificial Sequence
  Description of Artificial Sequence: Synthetic oligonucleotide
gaaatctataaaa

SEQ ID NO: 200 - DNA, 13 bp, Artificial Sequence
  Description of Artificial Sequence: Synthetic oligonucleotide
ggagtggcgagcc

SEQ ID NO: 201 - DNA, 33 bp, Artificial Sequence
  Description of Artificial Sequence: Synthetic oligonucleotide
ggagctctctggctaactagggaacccactgct

SEQ ID NO: 202 - DNA, 33 bp, Artificial Sequence
  Description of Artificial Sequence: Synthetic oligonucleotide
  modified_base (9)..(11): a, c, t, g, unknown or other
  modified_base (14)..(14): a, c, t, g, unknown or other
  modified_base (18)..(18): a, c, t, g, unknown or other
aaaggatannnttnaaanacacccaggagtcct

SEQ ID NO: 203 - DNA, 33 bp, Artificial Sequence
  Description of Artificial Sequence: Synthetic oligonucleotide
  modified_base (2)..(2): a, c, t, g, unknown or other
  modified_base (17)..(18): a, c, t, g, unknown or other
  modified_base (20)..(20): a, c, t, g, unknown or other
  modified_base (22)..(22): a, c, t, g, unknown or other
gnacctctctggctaannangnaacccactgtg

SEQ ID NO: 204 - DNA, 33 bp, Artificial Sequence
  Description of Artificial Sequence: Synthetic oligonucleotide
aaaggataatagtaaaagacaccaaggaagcct

SEQ ID NO: 205 - DNA, 10 bp, Artificial Sequence
  Description of Artificial Sequence: Synthetic oligonucleotide
gaaatctata

SEQ ID NO: 206 - DNA, 30 bp, Artificial Sequence
  Description of Artificial Sequence: Synthetic oligonucleotide
  modified_base (29)..(29): a, c, t, g, unknown or other
gctctctggctaactagggaacccactgng

SEQ ID NO: 207 - DNA, 33 bp, Artificial Sequence
  Description of Artificial Sequence: Synthetic oligonucleotide
aaaggataaatgtaaaagacaccaaggaagcct

SEQ ID NO: 208 - DNA, 10 bp, Artificial Sequence
  Description of Artificial Sequence: Synthetic oligonucleotide
tgcaaatgtt

SEQ ID NO: 209 - DNA, 10 bp, Artificial Sequence
  Description of Artificial Sequence: Synthetic oligonucleotide
gtggcgagcc

SEQ ID NO: 210 - DNA, 29 bp, Artificial Sequence
  Description of Artificial Sequence: Synthetic oligonucleotide
acagctacaaccatcccttcagacaggat

SEQ ID NO: 211 - DNA, 34 bp, Artificial Sequence
  Description of Artificial Sequence: Synthetic oligonucleotide
aaaggatagatgtaaaagacaccaaggaagcctt

SEQ ID NO: 212 - DNA, 10 bp, Artificial Sequence
  Description of Artificial Sequence: Synthetic oligonucleotide
agaccatcaa

SEQ ID NO: 213 - DNA, 64 bp, Artificial Sequence
  Description of Artificial Sequence: Synthetic oligonucleotide
ggtctctctggttagaccagatctgagcctgggagctctctggctaactagggaacccactgct

SEQ ID NO: 214 - DNA, 33 bp, Artificial Sequence
  Description of Artificial Sequence: Synthetic oligonucleotide
  modified_base (10)..(12): a, c, t, g, unknown or other
  modified_base (14)..(15): a, c, t, g, unknown or other
aaaggatagnnntnnaagacaccaaggaagcct

SEQ ID NO: 215 - DNA, 61 bp, Artificial Sequence
  Description of Artificial Sequence: Synthetic oligonucleotide
ctctctggttagaccagatctgagcctgggagctctctggctaactagggaacccactgct

SEQ ID NO: 216 - DNA, 27 bp, Artificial Sequence
  Description of Artificial Sequence: Synthetic oligonucleotide
tggaagggctaatttggtcccaaaaaa

SEQ ID NO: 217 - DNA, 37 bp, Artificial Sequence
  Description of Artificial Sequence: Synthetic oligonucleotide
ttggcagaactacacaccagggccagggatcagatat

SEQ ID NO: 218 - DNA, 27 bp, Artificial Sequence
  Description of Artificial Sequence: Synthetic oligonucleotide
accagatctgagcctgggagctctctg

SEQ ID NO: 219 - DNA, 27 bp, Artificial Sequence
  Description of Artificial Sequence: Synthetic oligonucleotide
tggaagggctaattcactcccaacgaa

SEQ ID NO: 220 - DNA, 27 bp, Artificial Sequence
  Description of Artificial Sequence: Synthetic oligonucleotide
accagatctgagcctgggagctctcgg

SEQ ID NO: 221 - DNA, 35 bp, Artificial Sequence
  Description of Artificial Sequence: Synthetic oligonucleotide
ttggcagaactacacaccagccagggatcagatat

SEQ ID NO: 222 - DNA, 31 bp, Artificial Sequence
  Description of Artificial Sequence: Synthetic oligonucleotide
ttggcagaactacacaccagggatcagatat

SEQ ID NO: 223 - DNA, 36 bp, Artificial Sequence
  Description of Artificial Sequence: Synthetic oligonucleotide
ttggcagaactacacaccaggccagggatcagatat

SEQ ID NO: 224 - DNA, 24 bp, Artificial Sequence
  Description of Artificial Sequence: Synthetic oligonucleotide
ttggcagaactacacaccaggtat

SEQ ID NO: 225 - DNA, 33 bp, Artificial Sequence
  Description of Artificial Sequence: Synthetic oligonucleotide
ttggcagaactacacacgccagggatcagatat

SEQ ID NO: 226 - DNA, 334 bp, Artificial Sequence
  Description of Artificial Sequence: Synthetic polynucleotide
ttggcagaactacacaccaggcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcctcgagtctagaggccgcaggaacccctagtgatggagttggccactccctctctgcgcgctcgctcgctcactgaggccgggcgaccaaaggtcgcccggggccagggatcagatat

SEQ ID NO: 227 - DNA, 297 bp, Artificial Sequence
  Description of Artificial Sequence: Synthetic polynucleotide
caatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcctcgagtctagaggccgcaggaacccctagtgatggagttggccactccctctctgcgcgctcgctcgctcactgaggccgggcgaccaaaggtcgcccggg

SEQ ID NO: 228 - DNA, 118 bp, Artificial Sequence
  Description of Artificial Sequence: Synthetic polynucleotide
ttggcagaactacacaccaggaaaggtcgcccgacgcccgggcggcctcagtgagcgagcgagcgcgcagctgcctgcaggacatgtgagcaaaaggccagcgccagggatcagatat

SEQ ID NO: 229 - DNA, 118 bp, Artificial Sequence
  Description of Artificial Sequence: Synthetic polynucleotide
ttggcagaactacacaccaggaaaggtcgcccgacgcccgggcggcctcagtgagcgggcgagcgcgcagctgcctgcaggacatgtgagcaaaaggccagcgccagggatcagatat

SEQ ID NO: 230 - DNA, 81 bp, Artificial Sequence
  Description of Artificial Sequence: Synthetic oligonucleotide
aaaggtcgcccgacgcccgggcggcctcagtgagcgagcgagcgcgcagctgcctgcaggacatgtgagcaaaaggccagc

SEQ ID NO: 231 - DNA, 81 bp, Artificial Sequence
  Description of Artificial Sequence: Synthetic oligonucleotide
aaaggtcgcccgacgcccgggcggcctcagtgagcgggcgagcgcgcagctgcctgcaggacatgtgagcaaaaggccagc

SEQ ID NO: 232 - DNA, 33 bp, Artificial Sequence
  Description of Artificial Sequence: Synthetic oligonucleotide
ttggcagaactacacaccagggccagggatcag

SEQ ID NO: 233 - DNA, 27 bp, Artificial Sequence
  Description of Artificial Sequence: Synthetic oligonucleotide
ttggcagaactacacaccagggatcag

SEQ ID NO: 234 - DNA, 32 bp, Artificial Sequence
  Description of Artificial Sequence: Synthetic oligonucleotide
ttggcagaactacacaccaggccagggatcag

SEQ ID NO: 235 - DNA, 29 bp, Artificial Sequence
  Description of Artificial Sequence: Synthetic oligonucleotide
ttggcagaactacacacgccagggatcag

SEQ ID NO: 236 - DNA, 31 bp, Artificial Sequence
  Description of Artificial Sequence: Synthetic oligonucleotide
ttggcagaactacacaccagccagggatcag

SEQ ID NO: 237 - DNA, 33 bp, Artificial Sequence
  Description of Artificial Sequence: Synthetic oligonucleotide
gtcacagcactccccaccagggcttgggggcaa

SEQ ID NO: 238 - DNA, 33 bp, Artificial Sequence
  Description of Artificial Sequence: Synthetic oligonucleotide
actaccctgggctctggtctgtaggtttgtagt

SEQ ID NO: 239 - DNA, 33 bp, Artificial Sequence
  Description of Artificial Sequence: Synthetic oligonucleotide
gaacttcatggccctggtgtggagtttagagtt

SEQ ID NO: 240 - DNA, 33 bp, Artificial Sequence
  Description of Artificial Sequence: Synthetic oligonucleotide
aggccaaagatgaaaaagacacccgagaaactt

SEQ ID NO: 241 - DNA, 32 bp, Artificial Sequence
  Description of Artificial Sequence: Synthetic oligonucleotide
taatgagacttgtacaagacaccacggggctt

SEQ ID NO: 242 - DNA, 32 bp, Artificial Sequence
  Description of Artificial Sequence: Synthetic oligonucleotide
tgcagaaaaatgtaaaagacaccttggaaaaa

SEQ ID NO: 243 - DNA, 21 bp, Artificial Sequence
  Description of Artificial Sequence: Synthetic oligonucleotide
tcggcagaactacacaccagg

SEQ ID NO: 244 - DNA, 12 bp, Artificial Sequence
  Description of Artificial Sequence: Synthetic oligonucleotide
ccaaggaagcct

SEQ ID NO: 245 - DNA, 21 bp, Artificial Sequence
  Description of Artificial Sequence: Synthetic oligonucleotide
ttggcagaactacacaccagg

SEQ ID NO: 246 - DNA, 39 bp, Artificial Sequence
  Description of Artificial Sequence: Synthetic oligonucleotide
ttggcagaactacacaccagggccaggggtcagatatcc

SEQ ID NO: 247 - DNA, 21 bp, Artificial Sequence
  Description of Artificial Sequence: Synthetic oligonucleotide
aaaggatagatgtaaaagaca

SEQ ID NO: 248 - DNA, 18 bp, Artificial Sequence
  Description of Artificial Sequence: Synthetic oligonucleotide
gccaggggtcagatatcc

SEQ ID NO: 249 - DNA, 18 bp, Artificial Sequence
  Description of Artificial Sequence: Synthetic oligonucleotide
gccagggatcagatatcc

SEQ ID NO: 250 - DNA, 22 bp, Artificial Sequence
  Description of Artificial Sequence: Synthetic oligonucleotide
aaaggatagatgtaaaagacac

SEQ ID NO: 251 - DNA, 53 bp, Artificial Sequence
  Description of Artificial Sequence: Synthetic oligonucleotide
aaagtagcaggaccacttctgcgctcggcccttccggctggccaaggaagcct

SEQ ID NO: 252 - DNA, 21 bp, Artificial Sequence
  Description of Artificial Sequence: Synthetic oligonucleotide
ttggcagaactccgcaccagg

SEQ ID NO: 253 - DNA, 21 bp, Artificial Sequence
  Description of Artificial Sequence: Synthetic oligonucleotide
  modified_base (13)..(13): a, c, t, g, unknown or other
aaaggatagatgnaaaagaca

SEQ ID NO: 254 - DNA, 16 bp, Artificial Sequence
  Description of Artificial Sequence: Synthetic oligonucleotide
ttggcagaactacaca

SEQ ID NO: 255 - DNA, 172 bp, Artificial Sequence
  Description of Artificial Sequence: Synthetic polynucleotide
gttacatcgaactggatctcaacagcggtaagatccttgatgtaacccactcgtgcacccaactgatcttcagcatctggtgctgtgctgacaactggtagttcctgccctacatcttccccagaagcgtgtccttcgacaacagcttcaacaacaaggtccaaggaagcct

SEQ ID NO: 256 - DNA, 18 bp, Artificial Sequence
  Description of Artificial Sequence: Synthetic oligonucleotide
  modified_base (12)..(12): a, c, t, g, unknown or other
gccaggggtcanatatcc

SEQ ID NO: 257 - DNA, 21 bp, Artificial Sequence
  Description of Artificial Sequence: Synthetic oligonucleotide
aaaggatagatgtaaaaaaca

(19) United States
(12) Patent Application Publication
(10) Pub. No.: US 2021/0060138 A1
(43) Pub. Date: Mar. 4, 2021

(54) CRISPR and LASER ART Eliminates HIV

(71) Applicants: Temple University - of the Commonwealth System of Higher Education, Philadelphia, PA (US); BOARD OF REGENTS OF THE UNIVERSITY OF NEBRASKA, Lincoln, NE (US)

(72) Inventors: Kamel Khalili, Bala Cynwyd, PA (US); Howard E. Gendelman, Omaha, NE (US); Benson Edagwa, Omaha, NE (US)

(73) Assignees: Temple University - of the Commonwealth System of Higher Education, Philadelphia, PA (US); BOARD OF REGENTS OF THE UNIVERSITY OF NEBRASKA, Lincoln, NE (US); BOARD OF REGENTS OF THE UNIVERSITY OF NEBRASKA, Lincoln, NE (US)

(21) Appl. No.: 16/812,140

(22) Filed: Mar. 6, 2020

Related U.S. Application Data
(60) Provisional application No. 62/814,591, filed on Mar. 6, 2019.

(51) Int. Cl. A61K 38/46; A61K 35/761; A61P 31/18; A61K 31/5365; A61K 31/7068; A61K 31/7076; A61K 31/505; A61K 31/7105

(52) U.S. Cl. CPC A61K 38/465; A61K 35/761; A61P 31/18; A61K 31/7105; A61K 31/7068; A61K 31/7076; A61K 31/505; A61K 31/5365

(57) ABSTRACT

Methods of eliminating a retrovirus from a subject utilize nanoformulated anti-retroviral compounds and gene editing agents. Compositions comprise at least one anti-retroviral compounds, at least one gene-editing agent, or combinations thereof.

### CROSS REFERENCE TO RELATED APPLICATIONS

This Application claims the benefit of U.S. Provisional Application 62/814,591 filed on Mar. 6, 2019. The entire contents of this application is incorporated herein by reference in its entirety.

### STATEMENT REGARDING FEDERALLY SPONSORED RESEARCH

This invention was made with government support under Grant Nos. P30MH092177, R01MH104147, R01N536126, R01N5034239, P01N543985, P30MH062261, P30AI078498, R01AG043540, P01DA028555, P01DA037830, R01MH110360, R01DA013137, R01NS087971, R24OD018546 and R01DA42706 awarded by the National Institutes of Health. The government has certain rights in this invention.

### SEQUENCE LISTING

The instant application contains a Sequence Listing which has been submitted electronically in ASCII format and is hereby incorporated by reference in its entirety. Said ASCII copy, created on Aug. 21, 2020, is named 052851-542001US_SL.txt and is 65,361 bytes in size.

### FIELD OF THE INVENTION

A combination therapy for the elimination and eradication of a retrovirus, for example, HIV, from an infected subject. In particular, the therapeutic approach utilizes long-acting slow effective release antiretroviral therapy (called LASER ART) and a gene editing agent.

### BACKGROUND

The elimination of the human immunodeficiency virus (HIV) from its viral reservoirs is a requirement for disease cure. Cure is defined as undetectable viremia measured in time periods of years in the absence of antiretroviral therapy (ART).

### SUMMARY

Embodiments of the invention are directed to a combination therapy comprising antiretroviral therapy (ART) along with gene editing.

In certain embodiments, a method of eradicating a retrovirus in a subject, comprises administering to a patient a composition comprising a therapeutically effective amount of at least one antiretroviral agent and/or a composition comprising a therapeutically effective amount of at least one gene editing agent. In certain embodiments, the antiretroviral or anti-viral agent is formulated as a long-acting slow effective release (LASER) antiretroviral agent. In certain embodiments, the at least one antiretroviral or anti-viral agent agent is nanoformulated. In certain embodiments, the at least one antiretroviral or anti-viral agent comprises: myristolyated dolutegravir, lamivudine, abacavir, rilpivirine or combinations thereof.

In certain embodiments, at least one antiretroviral agent is administered to the subject prior to administering the at least one gene editing agent. In certain embodiments, the at least one antiretroviral agent and at least one gene-editing agent are co-administered. In certain embodiments, the at least one antiretroviral agent and at least one gene-editing agent are administered sequentially.

In certain embodiments, the at least one gene editing agent comprises: an isolated nucleic acid sequence encoding a Clustered Regularly Interspaced Short Palindromic Repeat (CRISPR)-associated endonuclease/Cas (CRISPR/Cas) and at least one guide RNA (gRNA), the gRNA being complementary to a target nucleic acid sequence in a retroviral genome.

In certain embodiments, the CRISPR/Cas fusion protein comprises catalytically deficient Cas protein (dCas), orthologs, homologs, mutants variants or fragments thereof.

In certain embodiments, the at least one gRNA includes at least a first gRNA that is complementary to a target sequence in the integrated retroviral DNA; and a second gRNA that is complementary to another target sequence in the integrated retroviral DNA, whereby the intervening sequences between the two gRNAs are removed.

In certain embodiments, the isolated nucleic acid is included in at least one expression vector. In certain embodiments, the expression vector comprises a lentiviral vector, an adenoviral vector, or an adeno-associated virus vector. In certain embodiments the vector is an adeno-associated vector, e.g. AAV₉.

In certain embodiments, the retrovirus is a human immunodeficiency virus (HIV).

In certain embodiments, the target sequences comprise one or more nucleic acid sequences in HIV comprising: long terminal repeat (LTR) nucleic acid sequences, nucleic acid sequences encoding structural proteins, non-structural proteins or combinations thereof.

In certain embodiments, the sequences encoding structural proteins comprise nucleic acid sequences encoding: Gag, Gag-Pol precursor, Pro (protease), Reverse Transcriptase (RT), integrase (In), Env or combinations thereof. In certain embodiments, the sequences encoding non-structural proteins comprise nucleic acid sequences encoding: regulatory proteins, accessory proteins or combinations thereof. In certain embodiments, the regulatory proteins comprise: Tat, Rev or combinations thereof. In certain embodiments, the accessory proteins comprise Nef, Vpr, Vpu, Vif or combinations thereof.

In certain embodiments, a gRNA comprises at least one nucleic acid sequence set forth in Tables 1-5 or combinations of gRNAs.

In certain embodiments, a composition further comprises a therapeutically effective amount of a non-nucleoside reverse transcriptase inhibitor (NNRTI), and/or a nucleoside reverse transcriptase inhibitor (NRTI) and/or a protease inhibitor. In certain embodiments, the NNRTI comprises: etravirine, efavirenz, nevirapine, rilpivirine, delavirdine, or nevirapine. In certain embodiments, the NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

In certain embodiments, the pharmaceutical composition comprising a therapeutically effective amount of a nanoformulated long-acting slow effective release antiretroviral agent. In certain embodiments, the nanoformulated antiretroviral agent comprises: myristolyated dolutegravir, lamivudine, abacavir, rilpivirine or combinations thereof. In certain embodiments, the pharmaceutical composition comprises at least one an isolated nucleic acid sequence encoding a Clustered Regularly Interspaced Short Palindromic Repeat (CRISPR)-associated endonuclease; at least one isolated nucleic acid sequence encoding at least one guide RNA (gRNA) that is complementary to a target sequence in retroviral DNA; said isolated nucleic acid sequences being included in at least one expression vector. In certain embodiments the pharmaceutical composition comprise the gene-editing agent.

In certain embodiments, the integrated retroviral DNA is human immunodeficiency virus (HIV) DNA, and said at least one gRNA includes a first gRNA that is complementary to a first target sequence in the HIV DNA, and a second gRNA that is complementary to a second target sequence in the HIV DNA.

Other aspects are described infra.

Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” or “anti-retroviral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “eradication” of a retrovirus, e.g. human immunodeficiency virus (HIV), as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc. The presence or absence of the HIV virus can be determined via any means, such as for example, p24 detection or lack thereof, etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrastemal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “percent sequence identity” or having “a sequence identity” refers to the degree of identity between any given query sequence and a subject sequence.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

To “treat” a disease as the term is used herein, means to reduce the frequency or severity of at least one sign or symptom of a disease or disorder experienced by a subject. Treatment of a disease or disorders includes the eradication of a virus.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1G show the viral and immune profiles from sequential LASER ART and AAV₉-CRISPR-Cas9 treatments of HIV-1 infected humanized mice. FIG. 1A: After infection at week 0 and confirmation of VL, mice were administered 45 mg/kg nanoformulated myristoylated DTG (NMDTG), nanoformulated RPV (NRPV) and 40 mg/kg NM3TC, NMABC. Three weeks after the last LASER ART treatment, a single IV dose of AAV₉-CRISPR-Cas9 (10¹² GC units) was administered and left without antiretroviral drugs for an additional five weeks. FIG. 1B: Evaluation of human CD4⁺ T cell numbers in humanized mice by flow cytometry tests on days 0, 3, 5, 7, and 14 of infection. FIG. 1C: Viral load assessment by determining viral RNA copies in plasma at day-14 after HIV-1 NL₄₋₃ infection and prior to LASER ART treatment. FIG. 1D: Detection of human cells and viral infection in various tissues at day-14 after infection. Stains of human HLA-DR in lymph nodes, spleen, and lung show significant human immune cell reconstitution in infected animals. Replicate slides demonstrate HIV-1 p24⁺ stained cells in tissue sections. FIG. 1E: Detection of HIV-1 DNA by semi-nested real-time q-PCR assay in different tissues of HIV-1 infected animals at day-14 of infection. FIG. 1F: Evaluation of viral load shows that after administration of AAV₉-CRISPR-Cas9, two out of seven mice showed no evidence for viral rebound at week-14. Viral load in untreated animals remained high during the course of study. FIG. 1G: FACS analyses of human CD4⁺ T cells are shown with increased numbers in the LASER ART and AAV₉-CRISPR-Cas9 groups. A one-way ANOVA and Bonferroni's post-hoc tests for multiple comparisons and a two-tailed Student's t-test was used for statistical analyses in FIG. 1B. *P<0.05, **P<0.01.

FIGS. 2A-2C show the excision of the viral DNA fragments by CRISPR-Cas9 in tissues from HIV-1 infected humanized mice treated with LASER ART. FIG. 2A: Schematic illustration of HIV-1_{NL4-3} DNA highlighting the positions of gRNA LTR1 and gRNA Gag D target sites, their nucleotide compositions, and the three possible CRISPR-Cas9 induced break points leading to the excisions of various length of viral DNA fragments. Figure discloses SEQ ID NOS 151-153, respectively, in order of appearance. FIG. 2B: Total DNA from spleen, GALT, and kidney from three groups of animals were used for PCR genotyping using a set of primers derived from the 5′LTR, 3′LTR, and gag gene in reaction conditions that are calibrated for efficient amplification of short (less than 600 bp) or large DNA fragments. Predicted amplicons of 193 bp and 523 bp, which result from the excisions of DNA fragments between 5′ LTR to gag and gag to 3′LTR, respectively, were selected for DNA sequencing. The fragment of 396 bp represents both populations of full length LTRs, as well as the chimeric of both 5′ and 3′ LTR after excision of entire genome by gRNA LTR1/Cas9 and re-joining of the residual segments of cleaved 5′ LTR and 3′ LTRs. Several other fragments with closely similar size, caused by InDel mutations, were detected and further analyzed by sequencing. Single asterisks on top of the bands point to the specificity of the fragmental HIV DNA excision by CRISPR-Cas9 as verified by Sanger sequencing (also illustrated in FIGS. 12A-12C and 13A-13M). The double asterisk depicts non-specific amplification of unrelated DNA or randomly amplified segment of truncated HIV-1 sequence (also see FIGS. 14A-14F). FIG. 2C: Representative DNA sequences from each group were aligned to the reference LTR-Gag region of the HIV-1_{NL4-3} sequence. The positions and nucleotide compositions of targets for gRNAs LTR1 and GagD are shown in green, PAM in red and insertion sequences in yellow. Arrows highlighted positions of small and large deletions. Figure discloses the Spleen sequences as SEQ ID NOS 232, 190, 243-245, 244-245, 244-245, 244-245, 244, 190, 246-247, 247-248, 247, 249, 247-248, 247, 249, 247-248 and 250, the Galt sequences as SEQ ID NOS 232, 190, 245, 244-245, 244-245, 251, 245, 244-245, 244, 252, 244, 190, 246-248, 253, 248 and 247-248, and the Kidney sequences as SEQ ID NOS 232, 190, 254, 244-245, 255, 245, 244, 190, 246-247, 256, 247, 249, 257 and 249, all respectively, in order of appearance. Figure also discloses the first “Insertion” sequence as SEQ ID NO: 154 and the second “Insertion” sequence as SEQ ID NO: 155.

FIGS. 3A-3E show the detection of viral DNA and RNA in various tissues after sequential LASER ART and AAV₉-CRISPR-Cas9 treatments of infected humanized mice. (FIG. 3A) HIV-1 DNA and (FIG. 3D) HIV-1 RNA analyses using ultrasensitive semi-nested real-time PCR assays from spleen, bone marrow (BM), GALT, brain, liver, kidney, and lung from treatment groups. The data are expressed as total HIV-1 DNA (FIG. 3A) or HIV-1 RNA (FIG. 3D) copies/10⁶ human CD45⁺ cells. Two animals, #4346 and #4349 [shown by the red squares below the dashed lines (detection limit)], with dual treatments, showed sterilization of virus from all tissues analyzed. FIGS. 3B and 3C:Quantitative PCR showed complete elimination of signals corresponding to pol (FIG. 3B) and env (FIG. 3C) DNA sequences of HIV-1 in mice #4346 and #4349 (shown by red arrows). FIG. 3E: Representative results from RNAscope assay revealed the detection of single or clusters of brown dots corresponding to HIV-1 RNA in 5 pm-thick spleen sections of infected animals receiving either LASER ART or CRISPR-Cas9, but not both (#4346). E1 are representative spleen sections obtained from humanized mice infected with HIV-1 (controls); E2 are HIV-1 infected animals treated only with CRISPR-Cas9; E3 are HIV-1 infected LASER ART treated animals demonstrating viral rebound after cessation of therapy, and E4 are infected animals treated first with LASER ART followed by CRISPR-Cas9. E1-E4 are representative tissue sections taken from each of the animal groups. In these assays, we used the antisense V-HIV1-Clade-B targeting 854-8291 base pairs of HIV-1 as the probe. Human peptidylprolyl isomerase B (PPIB) was used as a positive control for all tissues analyzed. Images are 40× magnification. A one-way ANOVA and Bonferroni's post-hoc tests for multiple comparisons and a two-tailed Student's t-test was used for comparisons between two groups as in FIGS. 3A and 3D for statistical analyses. *P<0.05, **P<0.01, ***P<0.001.

FIGS. 4A-4G show the viral sterilization in HIV-1_{ADA} infected humanized mice in LASER ART and CRISPR-Cas9 (dual treated) by measures of viral, immune profile and excision profiles. FIG. 4A: The timeline of the experiment showing the temporal administration of LASER ART and CRISPR-Cas9 treatments, and animal sacrifice. FIG. 4B: The percentage of human CD4⁺ T-cells and (FIG. 4C) viral loads measured in the HIV-1 infected and HIV-1 infected and dual treated animal groups. Dual treated animals that showed no or viral rebound are illustrated. FIG. 4D: HIV-1 DNA analysis was performed using ultrasensitive semi-nested real-time q-PCR assays from spleen, GALT, liver, lung, brain and bone marrow from infected and infected and dual treated mice. The data are expressed as total HIV-1 DNA copies/10⁶ human CD45⁺ cells. Two animals, #3319 and #3336 (illustrated by the red squares) were below the dashed lines for virus detection as measured by plasma VL. These animals had no detectable viral DNA after dual treatments demonstrating viral sterilization from all analyzed tissues. A single animal (#3324) is illustrated by a half-red-black designation that had an undetectable VL but viral DNA was observed. FIG. 4E: Ultrasensitive ddPCR, with sensitivity of detecting 1-2 viral copies, was used in cross validation tests for viral DNA detections and performed in all tissues of infected and infected/dual treated animals. As a positive control, one animal each from the HIV-1 infected and HIV-1 and LASER ART groups are illustrated as open structures together. These were placed together with the six infected animals from the dual treatment group illustrated as closed structures. Dashed line represents the limit of detection. FIG. 4F: Agarose gel analyses of the PCR assay of DNA from various tissues of two animals with no rebound shows the presence of segments of HIV-1 LTR DNA and detection of a 121 bp amplicon, indicative of excision of a DNA fragment between the LTR and the gag gene (top). The histogram illustrates representative results from sequencing of the 121 bp fragment highlighting the position of the 5′ LTR breakpoint, and Gag and PAM trinucleotide on the GagD RNA. Figure discloses SEQ ID NO: 156. FIG. 4G: Splenocytes and bone marrow cells were isolated from HIV-1 infected mice with or without prior LASER ART and/or CRISPR-Cas9 treatments. These cells were then used in adoptive transfers performed in uninfected and drug naive mice. These transfer experiments performed in CD34⁺ HSC humanized mice were used to examine potential rebound from latent reservoirs not detected by standard ddPCR and nested PCR tests. In addition, as positive controls, two animals from an HIV-1 infected group and one from the LASER ART “alone” treatment group are shown as open circles and boxes. Five animals from the dual treatment group are illustrated as closed circles and boxes. Mice were sacrificed after 30 days and analyzed for plasma viral RNA using the Roche Ampliprep/Taqman-48 V2.0 detection assay. Virus was not detected in 2 “dual-treated” animals (#3319 and #3336, red circles and boxes below the dotted line for the cutoffs for viral detection) in all tests. This was used as the definition of “viral eradication” in these experiments. In contrast, virus was readily identified in all other infected and treated groups. A one-way ANOVA and Bonferroni's post-hoc tests for multiple comparisons and a two-tailed Student's t-test was used for comparisons between two groups as in FIGS. 4B and 4D for statistical analyses. *P<0.05.

FIGS. 5A-5F show the results from a study of viral and CD4⁺ T cell profiles from simultaneously treated HIV-1 infected Hu-PBL mice with LASER ART and AAV₉-CRISPR-Cas9. FIG. 5A: Study scheme illustrates time of human cells reconstitution, HIV-1 infection, LASER ART administration, AAV₉-CRISPR-Cas9 injection (50 μl of 2×10¹³ GC/ml), and time of sacrifice and flow cytometric evaluation of pan-human CD4⁺ T cells. FIG. 5B: Peripheral blood cells were assayed prior to and after (day 14) HIV-1 infection. FIG. 5C: Plasma viral load was detected using Roche ampliprep V2.0/Taqman 48 system from different mice groups. FIG. 5D: The DNA analysis from gag region from spleen tissues showed reduced HIV-1 in LASER ART alone which were further decreased in LASER ART plus AAV₉-CRISPR-Cas9 treated groups as compared to HIV-infected but untreated controls and the AAV₉-CRISPR-Cas9 group. FIG. 5E: HIV-1 RNA was analyzed using highly sensitive semi-nested real-time PCR assays from spleen samples of all four groups of mice at the end of the study (day-14 after infection). Significant decreases in HIV-1 RNA in LASER ART alone and LASER ART plus AAV₉-CRISPR-Cas9 treated groups compared to HIV-1 infected but untreated controls were observed. The data are expressed as the ratio of total HIV-1 RNA copies/10⁶ human CD45⁺ cells. FIG. 5F: Quantitative PCR of viral RNA from 14 days HIV-1 infected humanized mice. HIV-1 RNA was analyzed using ultrasensitive semi-nested real-time PCR assays from spleen, lymph node, bone marrow, lung, liver, and GALT obtained from HIV-1 infected humanized mice at day 14.

FIGS. 6A-6C show the combined effect of ART and CRISPR/Cas9 on HIV-1 infection of Jurkat T cell line. FIG. 6A: Experimental design and procedure. Jurkat cells were infected with HIV-1NL₄₋₃-GFP-P2A-Nef at multiplicity of infection (MOI) 0.001 and 0.01. Next, cells were divided into four groups: one control, DMSO treated and three treated with the cocktail composed of four antiretroviral drugs (ART) at the concentrations of 5×EC90 values (dolutegravir (DTG) 11.1 ng/ml, rilpivirine (RPV) 3.3 ng/ml, lamivudine (3TC) 17.2 μg/ml and abacavir (ABC) 8.3 μg/ml. Second set of experiments was performed using myristoylated, precursor antiretroviral drugs (LASER ART) similarly, at the doses 5×EC90 values (myristoylated dolutegravir (MDTG) 16.7 ng/ml, rilpivirine (RPV) 3.3 ng/ml, myristoylated lamivudine (M3TC) 32.9 μg/ml and myristoylated abacavir (MABC) 14.4 μg/ml). ART/LASER ART treatment was started at day 1, 3 or 5 after infection and fresh drugs were added daily. At day 6 of infection the drugs were removed to allow efficient lentiviral transduction of Cas9 and gRNAs LTR A and LTR B which was conducted at day 7. At day 8 antiretrovirals were added back and continued for another 4 days. Twelve days after HIV-1 infection cells were collected, genomic DNA was extracted and analyzed by PCR for CRISPR-Cas9 mediated cleavage of viral LTR sequences. FIG. 6B: Quantification of the level of infection at day 7. Cells were fixed with 2% PFA and FACS analysis was performed to measure GFP expressing population for HIV infection/replication in vitro. FIG. 6C: Similar to FIG. 6B with exception that cells were treated with modified ART.

FIGS. 7A-7E show the excision of HIV DNA fragment by CRISPR-Cas9 in ART treated T cells and Patient driven PBMCs. Results from standard PCRs of genomic DNA obtained from infected and treated T cells. The presence of full length LTR (357 bp) and truncated, CRISPR-Cas9 induced products (167 bp) was examined (FIGS. 7A and 7B) and aligned to HIV genome after Sanger sequencing. FIG. 7C: Results of the truncated PCR product obtained after purification from the agarose gel and TA cloning. gRNAs target sequences are shown in green, PAM sequences in red and PCR primers in blue. Below, a representative example of Sanger sequence tracing of truncated product. The HIV-1 LTR sequence was cleaved by Cas9 at target sites LTR A and LTR B and then joined together, resulting in deletion of 190 bp proviral DNA segment. The double cleaved/end-joined site is shown as a breaking point in red. Figure discloses SEQ ID NOS 157-161, 159 and 162, respectively, in order of appearance. FIG. 7D: PCR results of genomic DNA from PBMC's obtained from HIV positive individual. The presence of full length LTR (357bp) and truncated, CRISPR-Cas9 induced products (167bp) was examined. The cells were pretreated with ART for 5 (line 4.), 3 (line 3.) or 1 day (line 2.) or control, DMSO treated (line 1.). At day 6 drugs were removed and next day Cas9 and gRNAs were delivered by lentiviral transduction. At day-8 ART was resumed for another 4 days when cells were collected and processed same way like Jurkat cells above. FIG. 7E: Alignment of a representative Sanger sequencing results of the truncated PCR products obtained after purification from the agarose gel and TA cloning. gRNAs target sequences are shown in green, PAM sequences in red and PCR primers in blue. Below a representative examples of Sanger sequence tracing of truncated products. The HIV-1 LTR sequence was cleaved by Cas9 at target sites LTR A and LTR B and then joined together, resulting in deletion of 190bp proviral DNA segment. The double cleaved/end-joined site is shown as a breaking point in red. In the case of second clone a short: 5 bp deletion was detected at the cut site (in grey). Figure discloses SEQ ID NOS 163-166, 161, 159, 167, 161, 159, 168, 169 and 161, respectively, in order of appearance.

FIGS. 8A, 8B are flow cytometric evaluations of human leukocyte reconstitution in humanized mice. Peripheral blood of human stem cell reconstituted mice was assayed before and after (weeks 2, 6, 9, and 14) HIV-1 infection for the presence of human CD45⁺ (FIG. 8A) and CD3⁺ (FIG. 8B) cells. These experiments were performed to assess levels of humanization throughout the study. Numbers of human CD45⁺ and CD3⁺ cells were consistent within all the treated groups. These included animals not treated, treated with LASER ART or CRISPR-Cas9 alone or in combinations of LASER ART and CRISPR-Cas9. Notably, in the HIV-1 infected mice group, the numbers of CD45⁺ and CD3⁺ human cells in blood of mice were comparable to each of the treatment groups.

FIG. 9 shows the immunohistology of spleens from HIV-1 infected humanized mice. These mice were administered LASER ART or were left untreated. Animals were sacrificed at the time of CRISPR-Cas9 treatment to determine the presence of human CD4⁺ viral target T cells. Representative images are shown from mice infected with HIV-1NL₄₋₃ with or without LASER ART. Significant reductions in CD4⁺ T cells numbers (brown stained cells) are readily seen in the HIV-1-infected group compared to HIV-1 infected animals treated with LASER ART. Duplicate treatments groups demonstrate adequacy or randomization for CRISPR-Cas9 therapy.

FIG. 10 shows the verification of the presence of human cells in the spleens of humanized mice. PCR analysis of genomic DNA isolated from the spleens of humanized animals using primer sets specific to human and mouse (for a control) beta-globin genes.

FIG. 11 shows the excision of the viral DNA fragments by CRISPR-Cas9 in tissues from HIV-1 infected humanized mice with and without treatments with LASER ART. Results from standard PCRs of genomic DNA obtained from lungs, livers and brains of treated animals. The presence of full length LTR (396bp) and truncated, CRISPR-Cas9 induced products (193 bp for 5′LTR-gag and 523 bp for gag-3′LTR) were tested. *CRISPR-Cas9 mediated excision products. **Non-related.

FIGS. 12A-12C show the Sanger sequencing results of the truncated, CRISPR-Cas9 excised HIV-1 genomes. FIG. 12A: Representative examples of canonical, InDel free, CRISPR-Cas9 induced, double cleaved/end-joined HIV-1 genome truncations observed in majority of the tissues of AAV₉-CRISPR-Cas9/gRNA treated animals. On the left, result obtained from the spleen of mouse #4356 using 5′LTR-gag specific primers and on the right sequence from the spleen of mouse #4375 using gag-3′LTR specific amplification. Figure discloses SEQ ID NOS 170-171, respectively, in order of appearance. FIG. 12B: Verification of the presence of 41 bp insertion at the CRISPR-Cas9 mediated cleavage site in the viral sequence observed in GALT sample from mouse #4349. Figure discloses SEQ ID NO: 172. FIG. 12C: Sequence of the longer, 160 bp insertion found at the Cas9 cleavage site in the kidney sample from the same mouse #4349. Figure discloses SEQ ID NO: 173.

FIGS. 13A-13M show the Sanger sequencing tracing results of the truncated, CRISPR-Cas9 excised HIV-1 genomes. Representative examples of canonical, InDel free, CRISPR-Cas9 induced, double cleaved/end-joined HIV-1 genome truncations observed in majority of the tissues of AAV₉-Cas9/gRNA treated animals (FIGS. 13A (SEQ ID NO: 170), 13B (SEQ ID NO: 174): GALT; FIGS. 13C (SEQ ID NO: 170), 13D (SEQ ID NO: 175): Kidney; FIGS. 13E (SEQ ID NO: 170), 13F (SEQ ID NO: 176): Lung; FIGS. 13G (SEQ ID NO: 170), 13H (SEQ ID NO: 174): Liver and FIGS. 131 (SEQ ID NO: 170), 13J: Brain (SEQ ID NO: 174)). InDel mutation detected at the cleavage/end-joining sites in several tissues are shown in FIGS. 13K (SEQ ID NO: 177), 13L (SEQ ID NO: 178) for spleen, FIG. 13M (SEQ ID NO: 179) for kidney.

FIGS. 14A-14F show Sanger sequencing results of a few, non-related to CRISPR-Cas9, truncated HIV-1 amplicons detected in some of the samples. Sequences were aligned to HIV-1NL₄₋₃ sequence as a reference. The positions and nucleotide compositions of targets for gRNAs LTR1 and GagD are shown in green, PAMs in red. The sequencing data revealed lack of CRISPR-Cas9 specific cleavage (3 nucleotides from PAM) at the target sites LTR 1 (5′LTR in FIG. 14A for spleen lane 3 and 7, 3′LTR for kidney in FIG. 14C, lane 11, in FIG. 14D for lung lane 4 and FIG. 14F for brain, lane 9) or GagD (in FIG. 14C for kidney lane 7 and in FIG. 14E for liver lanes 2 and 16). Partial 3′LTR sequence was obtained for spleen lane 16 (FIG. 14B). FIG. 14A discloses the NL₄₋₃ sequences as SEQ ID NOS 180 and 181, the Lane 3 sequences as SEQ ID NOS 182 and 183, and the Lane 7 sequences as SEQ ID NOS 184 and 185, all respectively, in order of appearance. FIG. 14B discloses the NL₄₋₃ sequences as SEQ ID NOS 186 and 187 and the Lane 16 sequence as SEQ ID NO: 188, all respectively, in order of appearance. FIG. 14C discloses the NL₄₋₃ sequences as SEQ ID NOS 189-191, 186 and 192, the Lane 7 sequences as SEQ ID NOS 190 and 193, and the Lane 11 sequences as SEQ ID NOS 194, 186 and 195, all respectively, in order of appearance. FIG. 14D discloses the NL₄₋₃ sequence and the Lane 4 sequence as SEQ ID NOS 196 and 197, respectively, in order of appearance. FIG. 14E discloses the NL₄₋₃ sequences as SEQ ID NOS 189-190, 198-199, 186 and 200-201, the Lane 2 sequences as SEQ ID NOS 202 and 203, the Lane 16 top sequences as SEQ ID NOS 204, 198 and 205-206, and the Lane 16 bottom sequences as SEQ ID NOS 207-209 and 201, all respectively, in order of appearance. FIG. 14F discloses the NL₄₋₃ sequences as SEQ ID NOS 210-212, 186 and 213 and the Lane 9 sequences as SEQ ID NOS 214 and 215, all respectively, in order of appearance.

FIG. 15 shows the Cas9/gRNAs expression in the spleens of treated animals. Reverse transcription-PCR analysis of RNA extracted from spleens of treated animals to represent SaCas9 mRNA (top panels), single guide RNAs: LTR 1 (second row panels) and Gag D (third row panels) and a control beta-actin mRNA (bottom panels) were detected using primer sets specific to each target.

FIGS. 16A-16C show the hierarchical clustering analysis of the truncation efficiencies across different animals, treatments, tissues and HIV-1 gene segments. Probabilities are shown with the numbers as well as the heat-map intensities. Most similar groups are clustered together. Dendrograms indicate the hierarchy of clusters for each axis. FIG. 16A: Clustering of truncation efficiencies of different HIV-1 segments in different tissues under ART, CRISPR-Cas9 and ART plus CRISPR-Cas9 treatments. The clustering reveals the most similarity between ART plus CRISPR-Cas9-mediated editing in GALT, spleen and lung. FIG. 16B: Clustering of truncation efficiencies of different HIV-1 segments and qPCR data in different animals under ART, CRISPR-Cas9 and ART plus CRISPR-Cas9 treatments. The clustering scheme has recognized the similarity patterns and grouped the animals with the similar treatments under the same clusters. FIG. 16C: Clustering of truncation efficiencies in different tissues of the animals under the aforementioned treatments. Note that the animals with no rebound (treated with both LASER ART and AAV₉-CRISPR-Cas9) exhibit similar patterns in excision probabilities both across different HIV-1 segments and across different tissues. These analyses are later used in drawing the significance levels of combined treatment in viral genome eradication compared to the control groups. S1 refers to 5′ LTR-Gag and S2 refers to Gag-3′ LTR of the HIV-1 gene, respectively.

FIGS. 17A-17C show the Off target effect in cell model (FIG. 17A) of genomic DNA obtained from TZM-bl single cell clones: two controls (C1-2) and six Cas9/gRNA LTR 1+Gag D treated (E1-6). The presence of full length LTR −454/+43 (497 bp) was examined. Amplicons containing CRISPR-Cas9 specific InDel mutations at the LTR 1 target site in integrated HIV-1 LTR sequence are pointed by asterisks. Single asterisks indicate deletions, double asterisks insertions. FIG. 17B: Alignment of a representative Sanger sequencing results of HIV-1 LTR specific amplicons. The positions and nucleotide compositions of target for gRNA LTR1 is shown in green, PAM in red, sequence deletions in grey and sequence insertions in yellow, PCR primers in blue. Figure discloses SEQ ID NOS 216-219, 217, 220, 219, 217, 220, 219, 217, 220, 219, 217, 220, 219, 221, 220, 219, 222, 220, 219, 223, 220, 219, 223, 220, 219, 224, 220, 219, 225, 220, 219, 221, 220, 219, 221, 220, 219, 225, 220, 219, 223, 220, 219, 221, 220, 219, 221, 220, 219, 226, 220, 227, 219, 228, 220, 219, 229, 220, 230-231, 219, 229, 220 and 231, respectively, in order of appearance. FIG. 17C: Representative Sanger sequencing tracing of LTR 1 region of HIV-1 LTRs obtained for each single cell clone. The positions and nucleotide compositions of target for gRNAs LTR1 is shown in green, PAM in red, sequence deletions in grey. Figure discloses SEQ ID NOS 232, 232-236 and 235-236, respectively, in order of appearance.

FIGS. 18A-18F show representative Sanger sequencing tracing of predicted three Off target regions for gRNAs LTR 1 and Gag D obtained for each single cell clone. The positions and nucleotide compositions of Off target sites are shown in green, PAMs in red. Red squares point mismatched nucleotides comparing to target sequences. LTR 1 off target sites: TSC2 (FIG. 17A), TUB (FIG. 17B) and ch8 (FIG. 17C). Gag D off target sites: TACC2 (FIG. 17D), ADNP (FIG. 17E) and ch3 (FIG. 17F). No any InDel mutations at the predicted off target sites was detected. See also Tables 4 and 5. FIG. 18A discloses SEQ ID NOS 237 and 237, respectively, in order of appearance. FIG. 18B discloses SEQ ID NOS 238 and 238, respectively, in order of appearance. FIG. 18C discloses SEQ ID NOS 239 and 239, respectively, in order of appearance. FIG. 18D discloses SEQ ID NOS 240 and 240, respectively, in order of appearance. FIG. 18E discloses SEQ ID NOS 241 and 241, respectively, in order of appearance. FIG. 18F discloses SEQ ID NOS 242 and 242, respectively, in order of appearance.

FIGS. 19A-19C show the appearance of Somatic mutations in humanized mice. FIG. 19A: Sequence of NGS data analysis steps used for off target detection. FIG. 19B: Number of different types of somatic structural variations (SV) in each sample. Abbreviations: TRA: (Translocation) the number of translocations, INV: (Inversions) the number of inversions, DEL: (Deletion) the number of deletions, DUP: (Tandem duplication) the number of tandem duplications, INS: (Insertion) the number of insertions. FIG. 19C: The size of genomic regions affected by somatic CNVs in each sample.

FIGS. 20A-20D are Circos diagrams of the animals (FIG. 20A), #3539 ((LASER ART), (FIG. 20B) #4346 and, (FIG. 20C) #4349 (CRISPR-Cas9+LASER ART), and (FIG. 20D) #4356 (CRISPR-Cas9). The diagrams consist of seven rings. From outer to inner rings: (1) the outer circle (the first circle) is chromosome information. (2) The second ring represents the read coverage in histogram style. A histogram is the average coverage of a 0.5 Mbp region. (3) The third ring represents InDel density in scatter style. A black dot is calculated as InDel number in a range of 1 Mbp. (4) the fourth ring represents SNP density in scatter style. A green dot is calculated as SNP number in a range of 1 Mbp. (5) the fifth ring represents the proportion of homozygous SNP (orange) and heterozygous SNP (grey) in histogram style. A histogram is calculated from a 1 Mbp region. (6) The sixth ring represents the CNV inference. Red means gain, and green means loss. (7) The most central ring represents the SV inference in exonic and splicing regions. TRA (orange), INS (green), DEL (grey), DUP (pink) and INV (blue).

FIGS. 21A, 21B are an analysis of humanized mice tissues using highly sensitive ddPCR assay to detect HIV-1. Ultrasensitive droplet digital PCR (ddPCR) with sensitivity of detecting 1-2 copies was used to detect viral DNA in spleen of the infected animals belonging to 4 groups, control infected, LASER ART or AAV₉-CRISPR-CAs9 alone treated and dual treatment (LASER ART+Cas9) (FIG. 21A) and the various organs of the two mice with no viral rebound (FIG. 21B). Note that the two animals with double treatment group (group-4, #4346 and #4349) showed complete elimination of virus in spleen and the other tissues (Lung, liver, GALT, brain and Kidney) tested.

FIGS. 22A and 22B show a viral recovery assay using co-culture method. FIG. 22A: Splenocytes and bone marrow cells were isolated from HIV-1 infected mice with or without prior LASER ART and/or CRISPR-Cas9 treatments then co-cultivated with PHA/IL-2 stimulated human PBMCs. Cells were harvested 12 days post-cocultivation for HIV-1 DNA (FIG. 22A) and (FIG. 22B) RNA and looked to examine rebound virus using highly sensitive semi-nested real-time q-PCR assay. Data are expressed as total viral copies/10⁶ human CD45+ cells. Dual LASER ART and CRISPR-Cas9 treatments mice resulted in no detection of viral nucleic acids, which were also confirmed by reverse transcriptase assay of culture supernatants. Virus was detected in all other groups of animals.

FIG. 23 shows tissue analyses of HIV-1_{ADA} infected and treated humanized mice by RNAscope. RNAscope was used to detect viral RNA in spleens and demonstrating single brown dots or cluster of dots in 5-μm thick sections. The assays used antisense probeV-HIV-1-Calde-B targeting 854-8291 base pairs of the HIV-1 genome. Mouse #3319 which received LASER ART and AAV₉-CRISPR-Cas9, showed no viral detection signals. Viral RNA was detected in other 2 groups of humanized mice spleen (HIV-1_{ADA} infected and infected+LASER ART treated) as shown. The photomicrographs are representative images from each group. Human peptidyl Isomerase B (PPIB) was used as a positive control for every tissue analyzed. Images are 40× magnifications.

FIGS. 24A-24E show the detection of HIV-1_{ADA} DNA and RNA in spleen tissues in adoptively transferred humanized mice. Splenocytes and bone marrow cells were isolated from HIV-1 infected mice with or without prior LASER ART and or CRISPR-Cas9 treatments. These were for adoptive transfers into “new” CD34+ NSG-humanized mice. The intent was to perform cross disciplinary viral amplification from known infectious cell reservoirs. FIGS. 24A, 24B and 24C: HIV-1 DNA and (FIG. 24D) RNA analyses using ultrasensitive semi-nested real-time qPCR assays from spleen, bone marrow and lung tissues of adoptively transferred humanized mice. The data are expressed as total HIV-1 DNA or RNA copies/10⁶ human CD45⁺ cells. Four animals (splenocyte and bone marrow cells isolated and adoptively transferred from #3319 and #3336) mice (shown by red circles and squares below dotted line), showed no viral recovery. The above data was further confirmed using ultrasensitive ddPCR assay (with sensitivity of 1-2 copies), where the same four target adoptively transferred recipient animals showed no HIV-1 and (FIG. 24E) indicating complete elimination of virus. In mice from HIV-1_{ADA} infected with or without LASER ART treatment showed easily recovered virus in the spleen tissues. These results provide definitive testing of viral eradication in the two tested and the assayed mice (#3319 and #3336).

FIGS. 25A-25C show the excision of HIV proviral DNA by CRISPR-Cas9 in HIV_{ADA}-infected humanized mice. A much shorter fragment (193 bp) of excised HIV proviral DNA from the 5′LTR to gag region was amplified by nested-PCR in total genomic DNA extracted from various tissues of each humanized mice (#3324 and #3349) (FIG. 25A) along with the presence of SaCas9 DNA in each tissue (FIG. 25B). HIV excision was not detected in the humanized mouse treated with LASER ART only (#3357) even though a full length of HIV-1 LTR could be amplified abundantly to reveal the existence of HIV proviral DNA (FIG. 25C).

FIG. 26 shows liver tissue histology following therapy in humanized mice. Hematoxylin and eosin staining of representative sections from liver tissues in uninfected, HIV-1ADA-infected, infected and LASER ART treated and dual treated (LASER ART+AAV₉-CRISPR-Cas9) humanized mice at the endpoint of the study. Tissue pathology was not observed in LASER ART alone nor the dual treatment mice group. All images were captured at 10-× magnification.

FIG. 27 shows the gating strategy. Blood cells were first gated for mononuclear cells and lymphocytes using forward and side scattered panels (FSC and SSC). From the gated lymphocyte population, human CD45⁺ cells were re-gated in side-scatter panel. Gated human CD45⁺ mononuclear cells were assessed for expression of human CD3 (T cells) and CD19 (B cells). CD3⁺ T cells were further gated to assess the expression of CD4 and CD8 cells.

FIGS. 28A-28G are a series of graphs and stained tissue sections showing the viral and immune profiles following sequential LASER ART and AAV₉-CRISPR-Cas9 treatments of HIV-1 infected NSG-humanized mice. FIG. 28A: Human CD4⁺ T cells (%) in mice were enumerated by flow cytometry tests on days 0, 3, 5, 7, and 14 post-infection in HIV-1 infected animals (red line). Uninfected control animals are shown by the blue line. FIG. 28B: Representative data of virus in blood (plasma viral RNA copies/ml) are shown 14 days after HIV-1 infection (n=4). FIG. 28C: HIV-1 DNA was observed by semi-nested real-time qPCR in tissues of all HIV-1 infected animals 14 days after viral infection (n=4). FIG. 28D: Representative data sets of human HLA-DR in lymph nodes, spleen and lung demonstrating significant human cell reconstitution in all animals. Tissue sections stained for HIV-1p24 readily show large numbers of infected cells in tissues at day-14. FIG. 28E: The study scheme illustrates time points of infection and treatment. After confirmation for the establishment of the viral infection (shown in FIGS. 28A-28D) the rest 29 replicate humanized mice were subdivided into four groups. The first group (n=6) of mice were left untreated (HIV-1 control), the 2^{nd} group (n=6) received a single intravenous dose of AAV₉-CRISPR-Cas9 (10¹² GC units), nine weeks post-infection, the 3^{rd} group (n=10) were administered LASER ART by intramuscular injection after two weeks of viral infection, the 4^{th} (n=7) were given LASER ART (week 2-6 as in group 3) and three weeks after the last LASER ART treatment, a single intravenous dose of AAV₉-CRISPR-Cas9 (as in group-2). All mice remained without additional ART treatment for an additional five weeks. FIG. 28F: Flow cytometry tests of human CD4⁺ T cells are shown with increased numbers in the LASER ART and LASER ART+AAV₉-CRISPR-Cas9 group. FIG. 28G: Evaluation of viral load indicated that after administration of AAV₉-CRISPR-Cas9, two out of seven mice showed no evidence for viral rebound at 14 weeks. Viral load in untreated animals remained high during the course of study. One-way ANOVA and Bonferroni's post-hoc tests for multiple comparisons and two-tailed Student's t-test were used for statistical analyses in A. *P<0.05, **P<0.01.

FIGS. 29A-29H are a series of graphs showing the flow cytometric evaluation of human CD4⁺ T cells and Viral loads in individual humanized mice for HIV-1_{NL4-3} infected and/or treated groups. FIGS. 29A-29D: Peripheral blood of CD34-NSG-humanized mice were assayed before and after (2, 7, 9, and 14 weeks post HIV-1 infection (WPI) for the presence of human CD4⁺ cells from CD3⁺ gated populations throughout the study. FIG. 29A: Percentage of human CD4⁺ T cells followed a decreased pattern in all mice in the HIV-1 infected group. FIG. 29B: CD4⁺ T cell profile of HIV-1+LASER ART animals showed a decline in percentage of CD4⁺ T cells after two weeks of infection, after which the LASER ART treatment was followed for four additional weeks and the mice were then allowed for eight additional weeks without ART. FIG. 29C: Percentage of human CD4⁺ T cells were decreased in all mice in the HIV-1 and AAV₉-CRISPR-Cas9 infected group. FIG. 29D: CD4³⁰ T cells of HIV-1+LASER ART+AAV₉-CRISPR-Cas9 animals. Decreased percentages of CD4⁺ T cells were seen as early as two weeks of infection in all mice, after which the LASER ART treatment was administered for four weeks followed by AAV₉-CRISPR-Cas9 injection given at week-9. The mice were then followed for an additional five weeks. (FIG. 29E-29H: Plasma viral load of CD34⁺ NSG-hu mice was assayed after weeks 2, 7, 9, and 14 of HIV-1 infection for HIV-1 RNA to assess progression of disease using COBAS Ampliprep-Taqman-48 V2.0, the sensitivity of the assay after adjustment to dilution factor is (200 copies/ml). FIG. 29E: VL of six HIV-1 infected mice. FIG. 29F: VL profile of HIV-1+LASER ART animals. We observed a rebound of viral RNA at the study end in all 10 LASER ART treated animals, which corresponds to eight weeks after therapy interruption. FIG. 29G: VL of all six HIV-1 infected +AAV₉-CRISPR-Cas9 group. FIG. 29H: VL profile of HIV-1+LASER ART+AAV₉-CRISPR-Cas9 animals (n=7). Rebound of viral RNA was observed at the study end in five of seven dual treated animals, which corresponds to eight weeks-post therapy interruption, but observed no virus in two dual treated animals (#4346 and 4349).

FIGS. 30A-30C are a series of graphs stains and blots showing flow cytometric evaluations of human leukocyte reconstitution in blood and spleen of humanized mice. FIG. 30A: Peripheral blood of HSC reconstituted mice was assayed before and after (weeks 2, 7, 9, and 14) HIV-1 infection for the presence of human CD45⁺ (Left Panel) and CD3⁺ (Right Panel) cells. These experiments were performed to assess levels of humanization and percentage of total T cells (CD3 population) throughout the study. These included animals, group-1, HIV-1 infected (n=6), group-2, HIV-1 and CRISPR Cas9 (n=6), group-3, HIV-1 and LASER ART (n=10) and group-4, HIV-1 and LASER ART and CRISPRCas9 (n=7). Notably, in the HIV-1 infected mice group, the numbers of CD45⁺ and CD3⁺ human cells in blood of mice were comparable to each of the treatment groups. The colored triangles in the top identify the treatment time points during study (red-HIV-1 infection (no treatment, blue is LASER ART alone, black is CRISPR-Cas9 and green is LASER ART and CRISPR Cas9 injection to respective mice groups. We did not observe any statistically difference in any of the time points as compared to control animals. FIG. 30B: IHC of spleens from HIV-1 infected humanized mice. To determine the presence of human target T cells, spleens from HIV-1_{NL4-3} infected animals (untreated, LASER ART or both LASER ART +CRISPR/Cas9) were assessed for the presence of CD4⁺ T cells. Significant reductions in CD4⁺ T cells numbers (brown stained cells) are readily seen in the HIV-1-infected group compared to HIV-1 infected animals treated with LASER ART with/without CRISPR-Cas9. FIG. 30C: Verification of the presence of human cells in the spleens of humanized mice. PCR analysis of genomic DNA isolated from the spleens of humanized mice using primer sets specific to human and mouse (for a control) beta-globin genes.

FIGS. 31A-31C are blots and schematic illustrations showing the excision of the viral DNA fragments by CRISPR-Cas9 in tissues from HIV-1 infected humanized mice treated with LASER ART. FIG. 31A: Schematic illustration of HIV-1_{NL4-3} DNA highlighting the positions of gRNA LTR1 and gRNA GagD target sites, their nucleotide compositions, and the three possible CRISPR-Cas9 induced break points leading to the excisions of various lengths of viral DNA fragments. Figure discloses SEQ ID NOS 151-153, respectively, in order of appearance. FIG. 31B: Total DNA from spleen, GALT, and kidney from three groups of animals used for PCR genotyping with a set of primers derived from the 5′LTR, 3′LTR, and gag gene. Reaction conditions were calibrated for efficient amplification of short (less than 600 bp) or large DNA fragments. Predicted amplicons of 193 bp and 523 bp, which result from the excisions of DNA fragments between 5′LTR to Gag and Gag to 3′LTR, respectively, were selected for DNA sequencing. The fragment of 396 bp represents both populations of full length LTRs, as well as the chimeric of both 5′ and 3′LTR after excision of entire genome by gRNA LTR1/Cas9 and re-joining of residual segments of cleaved 5′LTR and 3′LTRs. Several other fragments with similar size, caused by InDel mutations, were detected and further analyzed by sequencing. Single asterisks above the bands point to the specificity of fragmental HIV DNA excision by CRISPR-Cas9 as verified by Sanger sequencing. The double asterisk depicts non-specific amplification of unrelated DNA or randomly amplified segment of truncated HIV-1 sequence. The dashed boxes show the excision of expected DNA fragments of the HIV-1 genome in the two animals with no viral rebound. FIG. 31C: Representative DNA sequences from each group were aligned to the reference LTR-Gag region of the HIV-1_{NL4-3} sequence. The positions and nucleotide compositions of targets for gRNAs LTR1 and GagD are shown in green, PAM in red, and insertion sequences in yellow. Arrows highlight positions of small and large deletions. Figure discloses the Spleen sequences as SEQ ID NOS 232, 190, 243-245, 244-245, 244-245, 244-245, 244, 190, 246-247, 247-248, 247, 249, 247-248, 247, 249, 247-248 and 250, the Galt sequences as SEQ ID NOS 232, 190, 245, 244-245, 244-245, 251, 245, 244-245, 244, 252, 244, 190, 246-248, 253, 248 and 247-248, and the Kidney sequences as SEQ ID NOS 232, 190, 254, 244-245, 255, 245, 244, 190, 246-247, 256, 247, 249, 257 and 249, all respectively, in order of appearance. Figure also discloses the first “Insertion” sequence as SEQ ID NO: 154 and the second “Insertion” sequence as SEQ ID NO: 155.

FIGS. 32A-32D show the efficiency of the proviral DNA excision by CRISPR-Cas9. FIG. 32A: Schematic of the locations of each gRNA and TaqMan probe and the possible excision outcomes. FIG. 32B: Absolute quantification of HIV-infected cells detected by digital-droplet PCR (ddPCR) using indicated primers and probes targeting LTR, Gag and Pol, respectively. Representative data collected from one HIV-infected humanized mouse of each group treated with LASER-ART (ART), LASER-ART plus CRISPR/Cas9 (ART/Cas9) or CRISPR/Cas9 only (Cas9). The genomic DNA extracted from a total of 50,000 cells including human and mouse cells was used as template for each ddPCR analysis. As shown in FIG. 32A, the reduction of Gag presents a deletion between 5′LTR and Gag or 5′LTR to 3′LTR, while a reduction in Pol represents the excision between Gag to 3′LTR or 5′LTR to 3′LTR. However, a single LTR will always remain to be detectable in all three conditions. Thus, we can use the ratio of Gag or Pol to LTR to estimate the excision efficiency. For example, in mouse #4349, the ratios of Gag/LTR and Pol/LTR are 19.7% (17 cells with detectable gag out of 76 cells with detectable LTR) and 19.4%, respectively, in the genomic DNA extracted from the spleen of the treated mice. Thus, the excision efficiencies of 5′LTR to Gag and Gag to 3′LTR were estimated to be about 80% for both (1 minus 19.7% or 100%-19.4%). In the spleen of the same mouse, the AAV9 transduction efficiency was calculated as high as 85% of the total population including both human graft and mouse host cells. In another mouse #4346, we demonstrated that the excision occurred mainly in Gag to 3′LTR because the ratio of Pol/LTR is 38.4% while Gag/LTR is 89.4%. Thus, the excision efficiency was estimated at 61.6% in 5′LTR to Gag and 10.6% in Gag to 3′LTR. Nonetheless, the presence of 2 LTRs in an uncut HIV proviral DNA was not considered in order to simplify the estimate. FIG. 32C: TaqMan probe and primers specific for saCas9, which was delivered by AAV9, were used to determine the AAV transduction efficiency and represented as the percentage of the cells containing saCas9 in a total of 50,000 cells including both human and mouse cell populations. FIG. 32D: Total human cell population in these 50,000 cells was measured using TaqMan probe and primers specific for human β-actin.

FIGS. 33A-33E show the detection of viral DNA and RNA at endpoint in various tissues after sequential LASER ART and AAV₉-CRISPR-Cas9 treatments in infected humanized mice. FIG. 33A: HIV-1 DNA and FIG. 33D: HIV-1 RNA analyses using ultrasensitive semi-nested real-time qPCR assays from spleen, bone marrow (BM), GALT, brain, liver, kidney, and lung from treatment groups described in FIGS. 28F-28G. The data represent each of the four groups: HIV-1 infected controls (n=5), HIV-1 infected and AAV₉-CRISPR-Cas9 treated (n=6), HIV-1 infected and LASER ART treated alone (n=4) and HIV-1 infected LASER ART and AAV₉-CRISPR-Cas9 treated mice (n=7). The data are expressed as total HIV-1 DNA (FIG. 33A) or HIV-1 RNA (FIG. 33D) copies/10⁶ human CD45⁺ cells. Two animals, #4346 and #4349 [shown by the red squares below the dashed lines (detection limit)], with dual treatments, showed sterilization of virus from all tissues analyzed. FIGS. 33B and 33C: Quantitative PCR showed complete elimination of signals corresponding to pol (FIG. 33B) and env (FIG. 33C) DNA sequences of HIV-1 in mice #4346 and #4349 (shown by red arrows). FIG. 33E: Representative results from RNAscope assay revealed the detection of single or clusters of brown dots corresponding to HIV-1 RNA in 5 μm-thick spleen sections of infected animals receiving either LASER ART or CRISPR-Cas9 alone, but not both (#4346). E1, humanized mice infected with HIV-1 (controls); E2, HIV-1 infected animals treated only with CRISPR-Cas9; E3, HIV-1 infected LASER ART treated animals demonstrating viral rebound after cessation of therapy; E4, infected animals treated first with LASER ART followed by CRISPR-Cas9. E1-E4 are representative tissue sections taken from each of the animal groups. In these assays, we used the antisense V-HIV1-Clade-B targeting 854-8291bp of HIV-1 as the probe. Images are 40× magnification. One-way ANOVA and Bonferroni's post-hoc tests for multiple comparisons and two-tailed Student's t-test were used for comparisons between two groups as in FIGS. 33A and 33D for statistical analyses. *P<0.05, **P<0.01, ***P<0.001.

FIG. 34A-34F show the viral sterilization in HIV-1_{ADA} infected humanized mice by LASER ART and CRISPR-Cas9 (dual treated) validated by viral, immune and excision profiling. FIG. 34A: The timeline of the experiment showing the temporal administration of LASER ART and CRISPR-Cas9 treatments, and animal sacrifice. FIG. 34B: The percentage of human CD4+ T cells and (FIG. 34C) viral loads measured in the HIV-1 infected (n=4), HIV-1 infected and LASER ART alone (n=7) and HIV-1 infected and dual treated (n=6) animal groups. Dual treated animals (n=6) that showed no (n=3) or viral rebound (n=3) in plasma are illustrated. FIG. 34D: HIV-1 DNA analysis was performed using ultrasensitive semi-nested real-time qPCR assays from spleen, GALT, liver, lung, brain and bone marrow from infected (n=4) and infected and dual treated mice (n=6). Three animals from the dual treated rebound group had very few bone marrow cells. Therefore, the data represent n=3 for the dual treated animals in the BM adoptive transfer studies. The data are expressed as total HIV-1 DNA copies/10⁶ human CD45⁺ cells. Two animals, #3319 and #3336 (illustrated by the red squares) were below the dashed lines for virus detection as measured by plasma VL. These animals had no detectable viral DNA after dual treatments demonstrating viral sterilization from all analyzed tissues. A single animal (#3324), as illustrated by a half-red-black designation, had an undetectable VL in plasma, but viral DNA was amplified in all the tissues analyzed. FIG. 34E: Ultrasensitive ddPCR, with sensitivity of detecting 1-2 viral copies, was used in cross validation tests for viral DNA detection and performed in tissues of infected and infected/dual treated animals. As a positive control, one animal each from the HIV-1 infected (open black structure) and HIV-1 and LASER ART (open green structure) groups are illustrated together. These were placed together with the six infected animals from the dual treatment group illustrated as closed structures (either black or red). Dashed line represents the limit of detection. FIG. 34F: Agarose gel analyses of the PCR assay of DNA from various tissues of two animals with no rebound shows the presence of segments of HIV-1 LTR DNA and detection of a 121 bp amplicon, indicative of excision of a DNA fragment between the LTR and the gag gene (top). The histogram illustrates representative results from sequencing of the 121 bp fragment highlighting the position of the 5′ LTR breakpoint, and Gag and PAM trinucleotide on the GagD RNA. Figure discloses SEQ ID NO: 156. FIG. 34G: An in vivo viral outgrowth assay was performed by adoptive transfer of splenocytes and bone marrow cells from infected and “virus eradicated” LASER ART CRISPR Cas9-treated mice to uninfected recipient CD34⁺ NSG-hu mice. These animals failed to show viral recovery after one month of examination by plasma viral RNA measurements. In confirmation assays performed as positive controls two animals from an HIV-1 infected groups (shown by open black circles for spleen and boxes for bone marrow) and an animal from a LASER ART treatment group are shown as open green circles (spleen) and box (bone marrow). All controls readily recovered virus. Five animals from the dual treatment group are illustrated as closed circles (spleen) and boxes (bone marrow). Virus was not detected in plasma from animals injected with splenocytes and bone marrow cells isolated from 2 “dual-treated” animals (#3319 and #3336, red circles and boxes). This was used as the definition of viral eradication in these experiments. One animal each from the HIV-1 and dual treated bone marrow injected group died so their data was not included. One-way ANOVA and Bonferroni's post-hoc tests for multiple comparisons and two-tailed Student's t-test were used for comparisons between two groups as in FIGS. 34B and 34D for statistical analyses. *P<0.05.

FIGS. 35A-35D are a schematic representation and a series of graphs showing viral load and CD4 T cells in HIV-1 infected and treated humanized mice. Mice were infected with 10⁴ TCID50 of HIV-1NL₄₋₃ followed by treatments with LASER ART, CRISPR-Cas9 or both. FIG. 35A. The study scheme shows the times of infection and treatments. After confirmation of viral infection, 29 infected humanized mice were subdivided into four groups. The first group (n=6, red) were left untreated (control), the second group (n=6, black) received a single intravenous (IV) dose of AAV₉-CRISPR-Cas9 (10¹² units), nine weeks after viral infection, the third group (n=10, blue) were administered LASER ART (NMDTG and NRPV at 45 mg/kg and NMABC and NM3TC at 40 mg/mg) by intramuscular (IM) injection two weeks after viral infection, the fourth (n=7, green) were given LASER ART (as in group 3) and three weeks after the last LASER ART treatment, a single IV dose of AAV₉-CRISPR-Cas9 was administered as in group 2. LASER ART treatment was ceased and after an additional five weeks, antiretroviral drug levels were assessed and were at or below the limit of quantitation <1 ng/ml (Table 8). FIG. 35B. Flow cytometry for human CD4 T cells are shown with increased numbers of CD4 counts in the LASER ART and dual LASER ART and CRISPR-Cas9 groups. FIG. 35C. Evaluation of plasma viral load indicated that after administration of AAV₉-CRISPR-Cas9, 2 of 7 mice showed no evidence for viral rebound at 14 weeks. FIG. 35D. Plasma viral load of individual animals for different treatment groups of humanized mice were assayed at 2, 7, 9, and 14 weeks of HIV-1 infection for HIV-1 RNA. Viral RNA levels were determined by the COBAS Ampliprep-Taqman-48 V2.0 assay with a sensitivity of 200 copies/ml once adjusted to the plasma dilution factor. Viral RNA rebound was observed at the study end in all 10 LASER ART treated animals. This corresponded to eight weeks after therapy interruption. Rebound was also observed at the study end in 5 of 7 dual-treated animals. Virus was not observed in two dual-treated animals (M4346 and M4349) and are highlighted in the red boxes.

FIGS. 36A-36D are a series of graphs demonstrating human CD4⁺ T cells in HIV-1 infected and treated humanized mice. FIGS. 36A-36D. Peripheral blood of humanized mice was assayed before and 2, 7, 9, and 14 weeks after HIV-1_{NL4-3} infection and the presence of human CD4⁺ cells from CD3⁺ gated populations were examined. FIG. 36A. Percentage of human CD4⁺ T cells followed a decreased pattern in all mice (n=6, red) in the HIV-1 infected group. FIG. 36B. Percentage of human CD4+ T cells were decreased in all mice (n=6, black) in the HIV-1 infected and AAV₉-CRISPR-Cas9 group. FIG. 36C. CD4⁺ T cell profile of HIV-1 infected and LASER ART animals (n=10, blue) showed a decline in CD4⁺ T cell numbers two weeks after viral infection. LASER ART was eliminated eight weeks after treatment. FIG. 36D. CD4⁺ T cells of HIV-1 infected and LASER ART and AAV₉-CRISPR-Cas9-treated animals (n=7, green). Decreased CD4⁺ T cell numbers were seen as early as two weeks after infection. At this time, LASER ART was administered for four weeks followed by AAV₉-CRISPR-Cas9 given at week 9. The mice were then followed for an additional five weeks. Restoration of CD4⁺ T cells was observed in both LASER ART and LASER ART and AAV₉-CRISPR-Cas9 treatment groups.

### DETAILED DESCRIPTION

Embodiments of the invention are directed in general to nanoparticle delivery of long-acting, slow effective release (LASER) antiretroviral therapy (ART) and gene editing technologies.

Briefly, the invention is based, in part, on the finding that treatment of HIV-1 infected humanized mice with CRISPR-Cas9 designed to edit the HIV-1 genome following two months treatment with the newly developed long-acting, slow effective release ART (LASER ART) eradicated HIV-1 infection in twenty-nine percent of infected animals with restored CD4⁺ T cells. Ultrasensitive nested and digital droplet PCR and RNA scope assays failed to detect HIV-1 in blood, spleen, lung, kidney, liver, gut-associated lymphoid tissue and brain. Excision of proviral DNA fragments spanning the LTRs and the Gag gene by CRISPR/Cas9, in the absence of any off target effects, along with the lack of viral rebound following cessation of ART with no progeny virus recovery verified HIV-1 eradication. Thus, the sequential application of antiretroviral agents and CRISPR-Cas9 therapies administered to HIV-1 infected humanized mice provided the first proof of concept that viral sterilization is possible.

LASER ART: Long-acting slow effective release ART (LASER ART) enable improved pharmacokinetic profiles and reservoir targeting. These antiretrovirals (ARVs) overcome limitations of current drugs associated with in vivo delivery and tissue penetrance. The gene editing agent also had improved delivery and improved the therapeutic index of the drugs.

Dolutegravir, lamivudine, abacavir and rilpivirine (DTG, 3TC, ABC and RPV respectively were transformed into long-acting drugs. Drug solubility, dissolution, metabolism, protein-binding, and excretion rates for each of the antiretroviral drugs were optimized and each were shown to influence the drug's half-life and biodistribution profiles. These studies provided the means to transform standard daily or twice-daily antiretroviral drugs into hydrophobic drug crystals to extend the drug's half-life and alter its solubility and metabolic patterns. The drugs were found to possess significant antiretroviral efficacy and high tolerability for conversion into a long-acting compound. Reversible chemical modification and polymer coating techniques were developed to convert each into a long-acting nanoformulation. Change of the antiretroviral drug (ARV) structure was made through reversible myristoylation of the native compound creating a water insoluble prodrug with commensurate crystal formation. When the drug crystals were packaged into a nanoparticle, they were rapidly taken up by human monocyte-derived macrophages (MDM), slowly released from the cells, and retained for a prolonged period inside the macrophage. These chemical and biological outcomes improved drug bioavailability and increased in vitro antiretroviral activity up to 100-fold. Pharmacokinetic and pharmacodynamic profiles were improved up to 10-fold over a native drug formulation, exhibiting broad tissue distribution and increased potency. The studies herein provide evidence that ARV conversion into a long-acting slow release formulation is readily achieved. As such, the drug-encased nanoparticles were employed as a “first-step” measure to facilitate drug penetrance into viral reservoirs to facilitate the actions of the excision Cas9 system.

Accordingly, in certain embodiments, the anti-retroviral agents are formulated into long-acting nanoformulated agents or compounds.

Gene Editing Agents: The application of Cas9 technology in eradicating HIV-1 reservoir, particularly targeting LTR, has been shown to be a promising strategy for treating and possibly curing AIDS. Hu, et al., PNAS 2014, 111:114616, disclosed that stable transfection of human cell cultures with plasmids expressing Cas9/gRNAs targeted to sites in the HIV-1 LTR successfully eradicated part and/or the entire HIV-1 genome without compromising host cell function. The targeted sites were termed LTR-A. LTR-B, LTR-C, and LTR-D. The targeting of two different sites in the LTR was particularly effective at producing the deletions sufficiently extensive to constitute the excision of all or substantially all of the proviral DNA sequence. The pre-existence of Cas9/gRNAs in cells also prevented new HIV-1 infection.

HIV and other retroviruses are highly mutable, so there is a need for a broader spectrum of Cas9/gRNA reagents and methods for targeting the integrated HIV genome. Of particular use would be Cas9/gRNA reagents that effectively target various genes in the viral genome, such as for example, structural genes of HIV, such as gag and pol; genes that encode ligands that allow for viral entry into cells, etc.

Accordingly, embodiments of the invention are directed to compositions and methods for the treatment and eradication of highly mutable and/or latent viruses from a host cell in vitro or in vivo. Methods of the invention may be used to remove viral or other foreign genetic material from a host organism, without interfering with the integrity of the host's genetic material. A nuclease may be used to target viral nucleic acid, thereby interfering with viral replication or transcription or even excising the viral genetic material from the host genome. The nuclease may be specifically targeted to remove only the viral nucleic acid without acting on host material either when the viral nucleic acid exists as a particle within the cell or when it is integrated into the host genome. Targeting the viral nucleic acid can be done using a sequence-specific moiety such as a guide RNA that targets viral genomic material for destruction by the nuclease and does not target the host cell genome. In some embodiments, a CRISPR/Cas nuclease and guide RNA (gRNA) that together target and selectively edit or destroy viral genomic material is used. The CRISPR (clustered regularly interspaced short palindromic repeats) is a naturally-occurring element of the bacterial immune system that protects bacteria from phage infection. The guide RNA localizes the CRISPR/Cas complex to a viral target sequence. Binding of the complex localizes the Cas endonuclease to the viral genomic target sequence causing breaks in the viral genome. Other nuclease systems can be used including, for example, zinc finger nucleases, transcription activator-like effector nucleases (TALENs), meganucleases, or any other system that can be used to degrade or interfere with viral nucleic acid without interfering with the regular function of the host's genetic material.

The compositions embodied herein, can be used to target viral nucleic acid in any form or at any stage in the viral life cycle. Together, with the combination of LASER-ART therapeutics, renders these compositions formidable in the treatment and/or prevention of infection by a retrovirues, e.g. HIV. The targeted viral nucleic acid may be present in the host cell as independent particles. In a preferred embodiment, the viral infection is latent and the viral nucleic acid is integrated into the host genome. Any suitable viral nucleic acid may be targeted for cleavage and digestion.

CRISPR/Cas Systems: The CRISPR-Cas system includes a gene editing complex comprising a CRISPR-associated nuclease, e.g., Cas9, and a guide RNA complementary to a target sequence situated on a DNA strand, such as a target sequence in proviral DNA integrated into a mammalian genome. The gene editing complex can cleave the DNA within the target sequence. This cleavage can in turn cause the introduction of various mutations into the proviral DNA, resulting in inactivation of HIV provirus. The mechanism by which such mutations inactivate the provirus can vary. For example, the mutation can affect proviral replication, and viral gene expression. The mutations may be located in regulatory sequences or structural gene sequences and result in defective production of HIV. The mutation can comprise a deletion. The size of the deletion can vary from a single nucleotide base pair to about 10,000 base pairs. In some embodiments, the deletion can include all or substantially all of the integrated retroviral nucleic acid sequence. In some embodiments the deletion can include the entire integrated retroviral nucleic acid sequence. The mutation can comprise an insertion, that is, the addition of one or more nucleotide base pairs to the pro-viral sequence. The size of the inserted sequence also may vary, for example from about one base pair to about 300 nucleotide base pairs. The mutation can comprise a point mutation, that is, the replacement of a single nucleotide with another nucleotide. Useful point mutations are those that have functional consequences, for example, mutations that result in the conversion of an amino acid codon into a termination codon or that result in the production of a nonfunctional protein.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains. Active DNA-targeting CRISPR-Cas systems use 2 to 4 nucleotide protospacer-adjacent motifs (PAMs) located next to target sequences for self versus non-self discrimination. ARMAN-1 has a strong ‘NGG’ PAM preference. Cas9 also employs two separate transcripts, CRISPR RNA (crRNA) and trans-activating CRISPR RNA (tracrRNA), for RNA-guided DNA cleavage. Putative tracrRNA was identified in the vicinity of both ARMAN-1 and ARMAN-4 CRISPR-Cas9 systems (Burstein, D. et al. New CRISPR-Cas systems from uncultivated microbes. Nature. 2017 Feb. 9; 542(7640):237-241. doi: 10.1038/nature21059. Epub 2016 December 22).

In embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In embodiments, the CRISPR/Cas system can be a type I, a type II, or a type III system. Non-limiting examples of suitable CRISPR/Cas proteins include Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csyl, Csy2, Csy3, Csel (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the invention also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9(K848A/K1003A/R1060A) (a.k.a. eSPCas9 1.1) are employed in the compositions. The invention is by no means limited to these variants, and also encompasses all Cas9 variants (Slaymaker, I. M. et al. Science. 2016 Jan. 1; 351(6268):84-8. doi: 10.1126/science.aad5227. Epub 2015 Dec. 1). The present invention also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

In one embodiment, the endonuclease is derived from a type II CRISPR/Cas system. In other embodiments, the endonuclease is derived from a Cas9 protein and includes Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Included are Cas9 proteins encoded in genomes of the nanoarchaea ARMAN-1 (Candidatus Micrarchaeum acidiphilum ARMAN-1) and ARMAN-4 (Candidatus Parvarchaeum acidiphilum ARMAN-4), CasY (Kerfeldbacteria, Vogelbacteria, Komeilibacteria, Katanobacteria), CasX (Planctomycetes, Deltaproteobacteria).

Embodiments of the invention also include a new type of class 2 CRISPR-Cas system found in the genomes of two bacteria recovered from groundwater and sediment samples. This system includes Cas1, Cas2, Cas4 and an approximately ˜980 amino acid protein that is referred to as CasX. The high conservation (68% protein sequence identity) of this protein in two organisms belonging to different phyla, Deltaproteobacteria and Planctomycetes, suggests a recent cross-phyla transfer. The CRISPR arrays associated with each CasX has highly similar repeats (86% identity) of 37 nucleotides (nt), spacers of 33-34 nt, and a putative tracrRNA between the Cas operon and the CRISPR array. Distant homology detection and protein modeling identified a RuvC domain near the CasX C-terminal end, with organization reminiscent of that found in type V CRISPR-Cas systems. The rest of the CasX protein (630 N-terminal amino acids) showed no detectable similarity to any known protein, suggesting this is a novel class 2 effector. The combination of tracrRNA and separate Cas1, Cas2 and Cas4 proteins is unique among type V systems, and phylogenetic analyses indicate that the Cas1 from the CRISPR-CasX system is distant from those of any other known type V. Further, CasX is considerably smaller than any known type V proteins: 980 aa compared to a typical size of about 1,200 amino acids for Cpf1, C2c1 and C2c3 (Burstein, D. et al., 2017 supra).

Another new class 2 Cas protein is encoded in the genomes of certain candidate phyla radiation (CPR) bacteria. This approximately 1,200 amino acid Cas protein, termed CasY, appears to be part of a minimal CRISPR-Cas system that includes Cas1 and a CRISPR array. Most of the CRISPR arrays have unusually short spacers of 17-19 nt, but one system, which lacks Cas1 (CasY.5), has longer spacers (27-29 nt). Accordingly, in some embodiments of the invention, the CasY molecules comprise CasY.1, CasY.2, CasY.3, CasY.4, CasY.5, CasY.6, mutants, variants, analogs or fragments thereof.

In some embodiments, the CRISPR/Cas-like protein can be derived from a wild type Cas protein or fragment thereof. In other embodiments, the CRISPR/Cas-like protein can be derived from modified Cas proteins. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In some embodiments, the CRISPR-associated endonuclease can be a sequence from another species, for example, other bacterial species, bacteria genomes and archaea, or other prokaryotic microorganisms. Alternatively, the wild type Cas9, CasX, CasY.1, CasY.2, CasY.3, CasY.4, CasY.5, CasY.6, ARMAN 1, ARMAN 4, sequences can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in GENBANK accession numbers KM099231.1 GI:669193757; KM099232.1 GI:669193761; or KM099233.1 GI:669193765. Alternatively, the Cas9, CasX, CasY.1, CasY.2, CasY.3, CasY.4, CasY.5, CasY.6, ARMAN 1, ARMAN 4, sequences can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of GENBANK accession numbers KM099231.1 GI:669193757; KM099232.1 GI:669193761; or KM099233.1 GI:669193765, or Cas9 amino acid sequence of PX330 or PX260 (Addgene, Cambridge, Mass.).

The wild type Cas9, CasX, CasY.1, CasY.2, CasY.3, CasY.4, CasY.5, CasY.6, ARMAN 1, ARMAN 4, sequences can be a mutated sequence. For example, the Cas9 nuclease can be mutated in the conserved HNH and RuvC domains, which are involved in strand specific cleavage. In another example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The sequences of Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof, can be modified to encode biologically active variants, and these variants can have or can include, for example, an amino acid sequence that differs from a wild type by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, polypeptides can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9, CasX, CasY.1, CasY.2, CasY.3, CasY.4, CasY.5, CasY.6, spCas, eSpCas, SpCas9, ARMAN 1, ARMAN 4 polypeptides. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Two nucleic acids or the polypeptides they encode may be described as having a certain degree of identity to one another. For example, a Cas9 protein and a biologically active variant thereof may be described as exhibiting a certain degree of identity. Alignments may be assembled by locating short Cas9 sequences in the Protein Information Research (PIR) site (pir.georgetown.edu), followed by analysis with the “short nearly identical sequences” Basic Local Alignment Search Tool (BLAST) algorithm on the NCBI website (ncbi.nlm.nih.gov/blast).

A percent sequence identity to Cas9 can be determined and the identified variants may be utilized as a CRISPR-associated endonuclease and/or assayed for their efficacy as a pharmaceutical composition. A naturally occurring Cas9 can be the query sequence and a fragment of a Cas9 protein can be the subject sequence. Similarly, a fragment of a Cas9 protein can be the query sequence and a biologically active variant thereof can be the subject sequence. To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chenna et al., Nucleic Acids Res. 31:3497-3500, 2003.

The Cas9 nuclease sequence can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks.

Guide RNA: A gRNA includes a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). In the present invention, the crRNA and tracrRNA can be expressed separately or engineered into an artificial fusion gRNA via a synthetic stem loop (AGAAAU) to mimic the natural crRNA/tracrRNA duplex. Such gRNA can be synthesized or in vitro transcribed for direct RNA transfection or expressed from U6 or H1-promoted RNA expression vector.

In the compositions of the present invention, each gRNA includes a sequence that is complementary to a target sequence in a retrovirus. The exemplary target retrovirus is HIV, but the compositions of the present invention are also useful for targeting other retroviruses, such as HIV-2 and simian immunodeficiency virus (SIV)-1.

Some of the exemplary gRNAs of the present invention are complimentary to target sequences in the long terminal repeat (LTR) regions of HIV. The LTRs are subdivided into U3, R and U5 regions. LTRs contain all of the required signals for gene expression, and are involved in the integration of a provirus into the genome of a host cell. For example, the basal or core promoter, a core enhancer and a modulatory region is found within U3 while the transactivation response element is found within R. In HIV-1, the U5 region includes several sub-regions, for example, TAR or trans-acting responsive element, which is involved in transcriptional activation; Poly A, which is involved in dimerization and genome packaging; PBS or primer binding site; Psi or the packaging signal; DIS or dimer initiation site.

Accordingly, in some embodiments a gRNA target sequence comprises one or more target sequences in an LTR region of an HIV proviral DNA and one or more targets in a structural gene and/or non-structural gene of the HIV proviral DNA. In other embodiments, a gRNA target sequence comprises one or more target sequences in an LTR region of an HIV proviral DNA and one or more targets in a structural gene. In another embodiment, a gRNA target sequence comprises one or more target sequences in an LTR region of an HIV proviral DNA and one or more targets in a non-structural gene of the HIV proviral DNA. In yet another embodiment, a gRNA target sequence comprises one or more target sequences in an HIV proviral a structural gene and one or more targets in a non-structural gene of the HIV proviral DNA. In yet another embodiment, a gRNA target sequence comprises one or more target sequences in an HIV proviral a non-coding gene and one or more targets in a coding gene of the HIV proviral DNA. In yet another embodiment a gRNA target nucleic acid sequence comprises one or more target nucleic acid sequences in a first gene and one or more target nucleic acid sequences in a second gene; or, one or more target nucleic acid sequences in a first gene and one or more target nucleic acid sequences in a third gene; or, one or more target nucleic acid sequences in a first gene and one or more target nucleic acid sequences in a second gene and one or more target nucleic acid sequences in a third gene; or, one or more target nucleic acid sequences in a second gene and one or more target nucleic acid sequences in a third gene or fourth gene; or, any combinations thereof. As can be seen, any combination of target nucleic acid sequences can be used and are only limited by the imagination of one of ordinary skill in the art.

In certain embodiments, target sequences comprise sequences within the U3, R, and U5 regions of the LTR. In certain embodiments the target sequences comprise one or more sequences from: LTR 1, LTR 2, LTR 3, LTR A, LTR B, LTR B′, LTR C, LTR D, LTR E, LTR F, LTR G, LTR H, LTR I, LTR J, LTR K, LTR L, LTR M, LTR N, LTR O, LTR P, LTR Q, LTR R, LTR S, AND LTR T. The compositions of the present invention include these exemplary gRNAs, but are not limited to them, and can include gRNAs complimentary to any suitable target site in the HIV LTRs.

Some of the exemplary gRNAs of the present invention target sequences in the protein coding genome of HIV. Sequences within the gene encoding the structural protein gag were found to be useful target sequences. gRNAs complementary to these target sequences include Gag A, Gag B, Gag C, and Gag D. Useful target sequences were also found within the gene encoding the structural protein pol. gRNAs complementary to these target sequences include Pol A and Pol B.

Examples of guide RNAs are shown in Tables 1-5. Accordingly, the compositions of the present invention include these exemplary gRNAs, but are not limited to them, and can include gRNAs complimentary to any suitable target site in the protein coding genes of HIV, including but not limited to those encoding the structural protein tat, and the accessory proteins vif, nef (negative factor) vpu (Virus protein U), vpr, and tev.

Guide RNA sequences according to the present invention can be sense or anti-sense sequences. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neiseria menigiditis requires 5′-NNNNGATT). The specific sequence of the guide RNA may vary, but, regardless of the sequence, useful guide RNA sequences will be those that minimize off-target effects while achieving high efficiency and complete ablation of the genomically integrated retrovirus, e.g. HIV. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites; avoiding transcription factor binding sites within the HIV LTR promoter (potentially conserved in the host genome); and WGS, Sanger sequencing and SURVEYOR assay, to identify and exclude potential off-target effects.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs. Combinations of gRNAs are especially effective when expressed in multiplex fashion, that is, simultaneously in the same cell. In many cases, the combinations produced excision of the HIV provirus extending between the target sites. The excisions are attributable to deletions of sequences between the cleavages induced by Cas9 at each of the multiple target sites. These combinations pairs of gRNAs, with one member being complementary to a target site in an LTR of the retrovirus, and the other member being complementary to a gRNA complementary to a target site in a structural gene of the retrovirus. Exemplary effective combinations include Gag D combined with one of LTR 1, LTR 2, LTR 3, LTR A, LTR B, LTR C, LTR D, LTR E, LTR F, LTR G; LTR H, LTR I, LTR J, LTR K, LTR L, LTR M; LTR N, LTR O, LTR P, LTR Q, LTR R, LTR S, or LTR T. Exemplary effective combinations also include LTR 3 combined with one of LTR-1, Gag A; Gag B; Gag C, Gag D, Pol A, or Pol B. In certain embodiments, a gRNA sequence has at least a 75% sequence identity to complementary target nucleic acid sequences encoding T LTR 1, LTR 2, LTR 3, LTR A, LTR B, LTR C, LTR D, LTR E, LTR F, LTR G; LTR H, LTR I, LTR J, LTR K, LTR L, LTR M; LTR N, LTR O, LTR P, LTR Q, LTR R, LTR S, or LTR T. The compositions of present invention are not limited to these combinations, but include any suitable combination of gRNAs complimentary to two or more different target sites in the HIV provirus.

In certain embodiments, a target nucleic acid sequence comprises one or more nucleic acid sequences in coding and non-coding nucleic acid sequences of the retroviral genome. The target nucleic acid sequence can be located within a sequence encoding structural proteins, non-structural proteins or combinations thereof. The sequences encoding structural proteins comprise nucleic acid sequences encoding: Gag, Gag-Pol precursor, Pro (protease), Reverse Transcriptase (RT), integrase (In), Env or combinations thereof. The sequences encoding non-structural proteins comprise nucleic acid sequences encoding: regulatory proteins e.g. Tat, Rev, accessory proteins, e.g. Nef, Vpr, Vpu, Vif or combinations thereof.

In certain embodiments, a gRNA sequence has at least a 75% sequence identity to complementary target nucleic acid sequences encoding Gag, Gag-Pol precursor, Pro, Reverse Transcriptase (RT), integrase (In), Env. Tat, Rev, Nef, Vpr, Vpu, Vif or combinations thereof.

In certain embodiments, a gRNA sequence is complementary to target nucleic acid sequences encoding Gag, Gag-Pol precursor, Pro, Reverse Transcriptase (RT), integrase (In), Env. Tat, Rev, Nef, Vpr, Vpu, Vif or combinations thereof.

In certain embodiments, gRNAs in single and multiplex configurations target the retroviral genome as well as the genes encoding receptors used by the virus to infect a cell, e.g. in the case of HIV, the receptor can be CCRS.

In some embodiments, the one or more isolated nucleic acids sequences are encoded by two or more constructs with one member directed toward a first retroviral target sequence, and the other member toward a second retroviral target sequence excises or eradicates the retroviral genome from an infected cell. Accordingly, the invention features compositions for use in inactivating a proviral DNA integrated into a host cell, including an isolated nucleic acid sequence encoding a CRISPR-associated endonuclease and one or more isolated nucleic acid sequences encoding one or more gRNAs complementary to a target sequence in HIV or another retrovirus. A second isolated nucleic acid sequence encoding a CRISPR-associated endonuclease and one or more isolated nucleic acid sequences encoding one or more gRNAs complementary to a target sequence encoding a receptor used by a virus to infect a cell. The isolated nucleic acid can include one gRNA, two gRNAs, three gRNAs etc. Furthermore, the isolated nucleic acid can include one or more gRNAs complementary to target sequences in the retrovirus and a second isolated nucleic acid can include one or more gRNAs complementary to target sequences encoding receptors used by the virus to infect a cell. Alternatively each isolated nucleic acid can include at least one gRNA complementary to a target virus sequence and at least one a gRNA complementary to target sequences encoding receptors used by the virus to infect a cell. One of ordinary skill in the art would only be limited by their imagination with respect to the various combinations of gRNAs.

Modified or Mutated Nucleic Acid Sequences: In some embodiments, any of the nucleic acid sequences may be modified or derived from a native nucleic acid sequence, for example, by introduction of mutations, deletions, substitutions, modification of nucleobases, backbones and the like. The nucleic acid sequences include the vectors, gene-editing agents, gRNAs, etc. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH,—N(CH₃)—O—CH₂ [known as a methylene(methylimino) or MMI backbone], CH₂ —O—N (CH₃)—CH₂, CH₂—N (CH₃)—N (CH₃)—CH₂ and O—N (CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me—C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N⁶ (6-aminohexyl)adenine and 2,6-diaminopurine. Kornberg, A., DNA Replication, W. H. Freeman & Co., San Francisco, 1980, pp75-77; Gebeyehu, G., et al. Nucl. Acids Res. 1987, 15:4513). A “universal” base known in the art, e.g., inosine may be included. 5-Me—C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1,2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N², 7-dimethyl guanosine; N², N²; 7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The isolated nucleic acid molecules of the present invention can be produced by standard techniques. For example, polymerase chain reaction (PCR) techniques can be used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Recombinant Constructs and Delivery Vehicles

Recombinant constructs are also provided herein and can be used to transform cells in order to express the isolated nucleic acid sequences embodied herein. A recombinant nucleic acid construct comprises promoter operably linked to a regulatory region suitable for expressing at least one tRNA, ribozyme, single guide RNA (sgRNA), gene editing agent or combinations thereof.

It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for Cas9 can be modified such that optimal expression in a particular organism is obtained, using appropriate codon bias tables for that organism.

Nucleic acids as described herein may be contained in vectors. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAGT™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). An example non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors is known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A.I. et al., J. Neurochem, 64: 487 (1995); Lim, F., et al., in DNA Cloning: Mammalian Systems, D. Glover, Ed. (Oxford Univ. Press, Oxford England) (1995); Geller, A.I. et al., Proc Natl. Acad. Sci.: U.S.A.:90 7603 (1993); Geller, A.I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M.G., et al., Nat. Genet. 8:148 (1994)].

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety.

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The anti-retroviral agents and/or the isolated nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infections. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding CRISPR/Cas and/or a guide RNA complementary to a target sequence of HIV, as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein. L-PEI has been used to efficiently deliver genes in vivo into a wide range of organs such as lung, brain, pancreas, retina, bladder as well as tumor. L-PEI is able to efficiently condense, stabilize and deliver nucleic acids in vitro and in vivo.

In some embodiments, delivery of vectors can also be mediated by exosomes. Exosomes are lipid nanovesicles released by many cell types. They mediate intercellular communication by transporting nucleic acids and proteins between cells. Exosomes contain RNAs, miRNAs, and proteins derived from the endocytic pathway. They may be taken up by target cells by endocytosis, fusion, or both. Exosomes can be harnessed to deliver nucleic acids to specific target cells.

The expression constructs of the present invention can also be delivered by means of nanoclews. Nanoclews are a cocoon-like DNA nanocomposites (Sun, et al., J. Am. Chem. Soc. 2014, 136:14722-14725). They can be loaded with nucleic acids for uptake by target cells and release in target cell cytoplasm. Methods for constructing nanoclews, loading them, and designing release molecules can be found in Sun, et al. (Sun W, et al., J. Am. Chem. Soc. 2014, 136:14722-14725; Sun W, et al., Angew. Chem. Int. Ed. 2015: 12029-12033.)

The nucleic acids and vectors may also be applied to a surface of a device (e.g., a catheter) or contained within a pump, patch, or any other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP).

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called “photothermal effect”. Because the near-infrared light can penetrate deeply into tissues, the surface of gold nanorod could be modified with nucleic acids for controlled release. When the modified gold nanorods are irradiated by near-infrared light, nucleic acids are released due to thermo-denaturation induced by the photothermal effect. The amount of nucleic acids released is dependent upon the power and exposure time of light irradiation.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more vectors encoding the isolated nucleic acids embodied herein. In some embodiments, the methods of the invention can be applied ex vivo. That is, a subject's cells can be removed from the body and treated with the compositions in culture to excise, and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD50 of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

Combination or Alternation Therapy

Accordingly, the invention features compositions which include therapeutically effective amounts of at least one antiretroviral agent administered sequentially or alternately or in conjunction with a composition for inactivating a proviral DNA integrated into a host cell. This composition comprises an isolated nucleic acid sequence encoding a CRISPR-associated endonuclease and one or more isolated nucleic acid sequences encoding one or more gRNAs complementary to a target sequence in HIV or another retrovirus.

In one embodiment, the antiretroviral agent comprises viral entry inhibitors, reverse transcriptase inhibitors, protease inhibitors, and immune-based therapeutic agents.

For example, when used to treat or prevent HIV infection, the antiretroviral agent or its prodrug or pharmaceutically acceptable salt can be administered in combination or alternation with another anti-HIV agent and/or a gene-editing agent embodied herein. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Combination therapy may be administered as (a) a single pharmaceutical composition which comprises an antiretroviral agent as described herein, at least one gene editing agent as described herein, and a pharmaceutically acceptable excipient, diluent, or carrier; or (b) two separate pharmaceutical compositions comprising (i) a first composition comprising an anti-retroviral agent as embodied herein and a pharmaceutically acceptable excipient, diluent, or carrier, and (ii) a second composition comprising at least one gene editing agents as embodied herein. The pharmaceutical compositions can be administered simultaneously or sequentially and in any order.

In use in treating or preventing viral disease, the antiretroviral(s) can be administered together with at least one gene editing agent as part of a unitary pharmaceutical composition. Alternatively, each can be administered apart from the other antiviral agents. In this embodiment, the antiretroviral(s) and the at least one at least one gene editing agent are administered substantially simultaneously, i.e. the compounds are administered at the same time or one after the other, so long as the compounds reach therapeutic levels for a period of time in the blood. In other embodiments, the antiretroviral agents are administered in one or more doses over a period of time followed by administration of the gene editing agents embodied herein.

The antiretroviral agents may be a nucleoside reverse transcriptase inhibitor, a nucleotide reverse transcriptase inhibitor, a non-nucleoside reverse transcriptase inhibitor, a protease inhibitor, an integrase inhibitor, a fusion inhibitor, a maturation inhibitor, or a combination thereof.

In certain embodiments, the at least one antiretroviral agent comprises: myristolyated dolutegravir, lamivudine, abacavir, rilpivirine or combinations thereof.

In certain embodiments, a composition comprises a therapeutically effective amount of a non-nucleoside reverse transcriptase inhibitor (NNRTI) and/or a nucleoside reverse transcriptase inhibitor (NRTI) ,and/or myristolyated dolutegravir, lamivudine, abacavir, rilpivirine analogs, variants or combinations thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof.

Examples of nucleoside reverse transcriptase inhibitors include zidovudine, didanosine, stavudine, zalcitabine, abacivir, emtricitabine, and lamivudine. Examples of non-nucleoside reverse transcriptase inhibitors include efavirenz, nevirapine, and delaviradine. Examples of protease inhibitors include indinavir, ritonavir, saquinavir, lopinavir, and nelfinavir. Examples of a reverse transcriptase inhibitor, an integrase inhibitor, a fusion inhibitor, and a maturation inhibitor are tenofovir, raltegravir, mariviroc, and bevirimat, respectively. In some aspects, the antiretroviral agents present in a nanoparticle include, ritonavir, lopinavir, and efavirenz, or efavirenz, abacavir, and lamivudine, or emtricitabine, tenofovir, and raltegravir.

In certain embodiments, the composition further comprises at least one or more protease inhibitors. In certain embodiments, a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

In certain embodiments, the compositions comprise an anti-retroviral agent, used in HAART, chemotherapeutic agents, activators of HIV transcription, e.g. PMA, TSA, and the like. Antiretroviral agents may include reverse transcriptase inhibitors (e.g., nucleoside/nucleotide reverse transcriptase inhibitors, zidovudine, emtricitibine, lamivudine and tenoifvir; and non-nucleoside reverse transcriptase inhibitors such as efavarenz, nevirapine, rilpivirine); protease inhibitors, e.g., tipiravir, darunavir, indinavir; entry inhibitors, e.g., maraviroc; fusion inhibitors, e.g., enfuviritide; or integrase inhibitors e.g., raltegrivir, dolutegravir. Antiretroviral agents may also include multi-class combination agents for example, combinations of emtricitabine, efavarenz, and tenofivir; combinations of emtricitabine; rilpivirine, and tenofivir; or combinations of elvitegravir, cobicistat, emtricitabine and tenofivir.

In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, or combinations thereof.

Some antiviral agents which can be used for combination therapy include agents that interfere with the ability of a virus to infiltrate a target cell. The virus must go through a sequence of steps to do this, beginning with binding to a specific “receptor” molecule on the surface of the host cell and ending with the virus “uncoating” inside the cell and releasing its contents. Viruses that have a lipid envelope must also fuse their envelope with the target cell, or with a vesicle that transports them into the cell, before they can uncoat.

There are two types of active agents which inhibit this stage of viral replication. One type includes agents which mimic the virus-associated protein (VAP) and bind to the cellular receptors, including VAP anti-idiotypic antibodies, natural ligands of the receptor and anti-receptor antibodies, receptor anti-idiotypic antibodies, extraneous receptor and synthetic receptor mimics. The other type includes agents which inhibit viral entry, for example, when the virus attaches to and enters the host cell. For example, a number of “entry-inhibiting” or “entry-blocking” drugs are being developed to fight HIV, which targets the immune system white blood cells known as “helper T cells”, and identifies these target cells through T-cell surface receptors designated “CRX4” and “CCR5”. Thus, CRX4 and CCR5 receptor inhibitors such as amantadine and rimantadine, can be used to inhibit viral infection, such as HIV.

Further antiviral agents that can be used in combination with the gene-editing agents embodied herein include agents which interfere with viral processes that synthesize virus components after a virus invades a cell. Representative agents include nucleotide and nucleoside analogues that look like the building blocks of RNA or DNA, but deactivate the enzymes that synthesize the RNA or DNA once the analogue is incorporated. Acyclovir is a nucleoside analogue, and is effective against herpes virus infections. Zidovudine (AZT), 3TC, FTC, and other nucleoside reverse transcriptase inhibitors (NRTI), as well as non-nucleoside reverse transcriptase inhibitors (NNRTI), can also be used. Integrase inhibitors can also be used.

Once a virus genome becomes operational in a host cell, it then generates messenger RNA (mRNA) molecules that direct the synthesis of viral proteins. Production of mRNA is initiated by proteins known as transcription factors, and certain active agents block attachment of transcription factors to viral DNA.

Other active agents include antisense oligonucleotides and ribozymes (enzymes which cut apart viral RNA or DNA at selected sites). HIV include protease enzymes, which cut viral protein chains apart so they can be assembled into their final configuration. Protease inhibitors are another type of antiviral agent that can be used in combination with the inhibitory compounds described herein. The final stage in the life cycle of a virus is the release of completed viruses from the host cell.

Still other active agents function by stimulating the patient's immune system. Interferons, including pegylated interferons, are representative compounds of this class.

In certain embodiments, the anti-viral or antiretroviral agent comprises therapeutically effective amounts of: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

The immune-modulating molecules comprise, but are not limited to cytokines, lymphokines, T cell co-stimulatory ligands, etc. An immune-modulating molecule positively and/or negatively influences the humoral and/or cellular immune system, particularly its cellular and/or non-cellular components, its functions, and/or its interactions with other physiological systems. The immune-modulating molecule may be selected from the group comprising cytokines, chemokines, macrophage migration inhibitory factor (MIF; as described, inter alia, in Bernhagen (1998), Mol Med 76(3-4); 151-61 or Metz (1997), Adv Immunol 66, 197-223), T-cell receptors or soluble MHC molecules. Such immune-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3(1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin O and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

Any of the above-mentioned compounds can be used in combination therapy with the gene editing agents embodied herein. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks. The therapeutic agents may be administered under a metronomic regimen, e.g., continuous low-doses of a therapeutic agent.

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue specific antibody. The liposomes will be targeted to and taken up selectively by the organ.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

When treating viral infections, an effective amount of the inhibitory compound is an amount sufficient to suppress the growth and proliferation of the virus. Viral infections can be prevented, either initially, or from re-occurring, by administering the compounds described herein in a prophylactic manner. Preferably, the effective amount is sufficient to obtain the desired result, but insufficient to cause appreciable side effects.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The effective dose can vary, depending upon factors such as the condition of the patient, the severity of the viral infection, and the manner in which the pharmaceutical composition is administered. The effective dose of compounds will of course differ from patient to patient, but in general includes amounts starting where desired therapeutic effects occur but below the amount where significant side effects are observed. For human patients, the effective dose of typical compounds generally requires administering the compound in an amount of at least about 1, often at least about 10, and frequently at least about 25 μg/24 hr/patient. The effective dose generally does not exceed about 500, often does not exceed about 400, and frequently does not exceed about 300 μg/24 hr/patient. In addition, administration of the effective dose is such that the concentration of the compound within the plasma of the patient normally does not exceed 500 ng/mL and frequently does not exceed 100 ng/mL.

The compounds, when employed in effective amounts in accordance with the method described herein, are effective at eliminating the retrovirus from the subject.

In some embodiments, the compositions may be formulated as a topical gel, for example, to treat a melanoma after excision, or an autoimmune condition expressed as a skin condition e.g. pemphigus. In some embodiments, the compositions can be formulated as a nanoparticle encapsulating a nucleic acid.

A subject is effectively treated whenever a clinically beneficial result ensues. This may mean, for example, a complete resolution of the symptoms of a disease, a decrease in the severity of the symptoms of the disease, or a slowing of the disease's progression. These methods can further include the steps of a) identifying a subject (e.g., a patient and, more specifically, a human patient) who has a certain disease to be treated; and b) providing to the subject the compositions comprising at least one anti-viral or anti-retroviral agent and/or a composition comprising the gene editing agents embodied herein.

In methods of treatment of HIV-1 infection, a subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present invention, one can first determine whether a patient has a latent HIV infection, and then make a determination as to whether or not to treat the patient with one or more of the compositions described herein. In some embodiments, the methods can further include the step of determining the nucleic acid sequence of the particular HIV harbored by the patient and then designing the guide RNA to be complementary to those particular sequences. For example, one can determine the nucleic acid sequence of a subject's LTR U3, R or U5 region, or pol, gag, or env genes etc., and then design or select one or more gRNAs to be precisely complementary to the patient's sequences. The novel gRNAs provided by the present invention greatly enhance the chances of formulating an effective treatment. The gRNAs targeted to nucleic acid sequences encoding a receptor used by a virus to infect a cell would prevent further infection.

In methods of reducing the risk of HIV infection, a subject at risk for having an HIV infection can be, for example, any sexually active individual engaging in unprotected sex, i.e., engaging in sexual activity without the use of a condom; a sexually active individual having another sexually transmitted infection; an intravenous drug user; or an uncircumcised man. A subject at risk for having an HIV infection can be, for example, an individual whose occupation may bring him or her into contact with HIV-infected populations, e.g., healthcare workers or first responders. A subject at risk for having an HIV infection can be, for example, an inmate in a correctional setting or a sex worker, that is, an individual who uses sexual activity for income employment or nonmonetary items such as food, drugs, or shelter.

The methods disclosed herein can be applied to a wide range of species, e.g., humans, non-human primates (e.g., monkeys), horses or other livestock, dogs, cats, ferrets or other mammals kept as pets, rats, mice, or other laboratory animals.

The methods of the invention can be expressed in terms of the preparation of a medicament. Accordingly, the invention encompasses the use of the agents and compositions described herein in the preparation of a medicament. The compounds described herein are useful in therapeutic compositions and regimens or for the manufacture of a medicament for use in treatment of diseases or conditions as described herein.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. An effective amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

Kits

The compositions described herein can be packaged in suitable containers labeled, for example, for use as a therapy to treat a subject having a viral infection, for example, an HIV infection or a subject at risk of contracting for example, an HIV infection. The containers can include a composition comprising at least one anti-viral or anti-retroviral agent;, a gene-editing agent and one or more of a suitable stabilizer, carrier molecule, flavoring, and/or the like, as appropriate for the intended use. In other embodiments, the kit further comprises one or more therapeutic reagents that alleviate some of the symptoms or secondary bacterial infections that may be associated with an HIV infection. Accordingly, packaged products (e.g., sterile containers containing one or more of the compositions described herein and packaged for storage, shipment, or sale at concentrated or ready-to-use concentrations) and kits, including at least one composition of the invention, and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

### Example 1

### Combination of CRISPR-Cas9 and Long-Acting Antiretroviral Therapy Eliminates HIV-1 Infection in Humanized Mice

A cure of HIV-1 infection has been stalled by the absence of a strategy for effective eradication of HIV-1 from the infected tissues and cells serving as viral reservoirs. As such, rebound uniformly occurs after cessation of currently used antiretroviral therapy, ART, that potently controls viral replication but does not eliminate proviral DNA.

Two approaches were combined, herein, to examine whether LASER ART and CRISPR-Cas9 treatments could provide combinatorial benefit for viral elimination. In this study, elimination of replication competent HIV-1 in an experimental model of human infectious disease, was demonstrated. Viral clearance was achieved from HIV-1 infected spleen and lymphoid tissues as well as a broad range of solid organs from documented prior infected humanized mice treated with LASER ART and AAV₉-CRISPR-Cas9. This was confirmed in those mice using ultrasensitive HIV-1 nucleic acid detection methods by the absence of post-treatment viral rebound; and by the inability to transfer virus from those infected and dual-treated mice to replicate uninfected untreated mice. It was concluded that viral elimination by a combination of LASER ART and gene editing strategy is possible.

Materials and Methods

Cell culture. TZM-bl reporter cell line (AIDS Reagent Program, Division of AIDS, NIAID, NIH, Bethesda, MD) and HEK-293T cells were cultured in DMEM high glucose complemented with 10% FBS and gentamicin (10 μg/ml). Jurkat, Clone E6 cells were purchased from ATCC (TIB-152™) and were cultured in RPMI medium containing 10% FBS and gentamicin (10 ug/ml). Patient blood samples were obtained through the Comprehensive NeuroAlDS Center (CNAC) Clinical Core (Temple University, Philadelphia, Pa., USA). PBMCs were isolated from human peripheral blood by density gradient centrifugation using Ficoll-Paque reagent. Blood sample volume was adjusted to 30 ml with HBSS buffer, gently layered on 15 ml of Ficoll-Paque cushion and centrifuged for 30 minutes at 1500 RPM. PBMCs containing layer was collected, washed 3 times in HBSS buffer and counted. Cells were incubated with PHA (5 μg/ml) for 24 h and then cultured in RPMI with 10% FBS and gentamicin (10 ug/ml) supplemented with human rIL-2 at a concentration of 30 ng/ml (STEMCELL Tech.). Fresh media was added every 2-3 days.

Cell culture reagents. 4-(2-Hydroxyethyl)-1-piperazineethanesulfonic acid (HEPES) buffer and ciprofloxacin were purchased from Sigma-Aldrich, St. Louis, Mo. Diethyl ether, endotoxin-free water, gentamicin, acetonitrile (ACN), methanol, KH₂PO₄, bovine serum albumin (BSA), Triton X-100, LC-MS-grade water, and TRIzol reagent were purchased from Fisher Scientific, San Diego, Calif., The TZM-bl reporter cell line (AIDS Reagent Program, Division of AIDS, NIAID, NIH, Bethesda, Md.) and HEK-293T cells (the American Type Culture Collection (ATCC), Manassas, Va.) were cultured in high glucose DMEM supplemented with 10% FBS and gentamicin (10 μg/ml). Jurkat (Clone E6-1, TIB-152™) cells were purchased from ATCC and cultured in Roswell Park Memorial Institute (RPMI) medium containing 10% FBS and gentamicin (10 μg/ml) (Sigma-Aldrich, St. Louis, Mo.). PBMCs were isolated from leukopaks by gradient centrifugation on Ficoll-Paque for 30 minutes at 600 g. PBMCs collected from the buffy coat were stimulated with PHA (5 μg/ml) for 24 h in RPMI with 10% FBS and gentamicin (10 μg/ml) supplemented with human recombinant interleukin-2 (rIL-2) at a concentration of 30 ng/ml ((STEMCELL Technologies, Seattle, Wash.). Fresh media was exchanged every 2-3 days.

In vitro Infection: HEK-293T cells were transfected using CaPO₄ precipitation method in the presence of chloroquine (50 μM) with 30 μg of pNL₄₋₃-EGFP-P2A-Nef plasmid (13)/2.5×10⁶ cells/100 mm dish. Next day, medium was replaced; and 24 h and 48 h later supernatants were collected, clarified at 3000 RPM for 10 minutes, filtered through 0.45 um filter, and concentrated by ultracentrifugation for 2 h with 20% sucrose cushion (25). Viral pellets were resuspended in HBSS by gentle agitation overnight, aliquoted, and tittered in Jurkat cells by FACS for GFP expression. Jurkat cells were infected by spinoculation for 1.5 h (26), 32° C. in 500 μl inoculum containing 8 μg/ml polybrene then resuspended and left for 4 h then 500 μl of growth medium was added. Next day, cells were washed 3 times with PBS and re-suspended in growth medium.

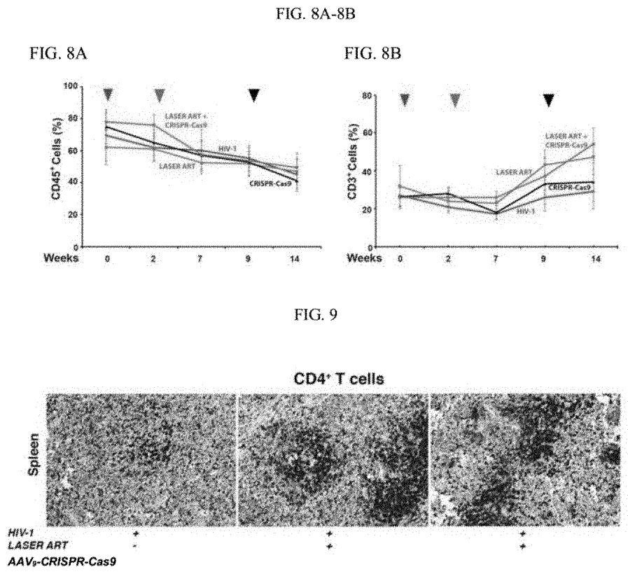

Generation of humanized NSG mice: NOD/scid-IL-2Rγ_{c}^{null} (NSG) mice were obtained from the Jackson Laboratories, Bar Harbor, Me. and bred under specific pathogen-free conditions in accordance with the ethical guidelines for care of laboratory animals at the University of Nebraska Medical Center (UNMC) set forth by the National Institutes of Health. CD34⁺ cells were obtained from human cord blood and enriched using immune-magnetic beads (CD34⁺ selection kit; Miltenyi Biotec Inc., Auburn, Calif., USA). CD34⁺ cell purity was >90% by flow cytometry. Cells were transplanted into newborn mice irradiated at 1 Gy using a C9 cobalt 60 source (Picker Corporation, Cleveland, Ohio, USA). Cell suspension was delivered by intrahepatic (i.h.) injection of 10⁴ cells/mouse in 20 μl phosphate-buffered saline (PBS) with a 30-gauge needle. Humanization of the animals was affirmed by flow cytometry (21, 27) for CD45 and CD3 staining of blood immune cells shown in FIGS. 8A, 8B.

HIV-1 infection of CD34 ⁺ humanized mice. NSG (NOD.Cg-Prkdc_{scid}Il2rgt^{mlWjl}/SzJ) mice were obtained from the Jackson Laboratories, Bar Harbor, Me and bred under specific pathogen-free conditions at the University of Nebraska Medical Center (UNMC) in accordance with the ethical guidelines set forth by the National Institutes of Health for care of laboratory animals. CD34⁺ HSC were enriched from human cord blood or fetal liver cells using immune-magnetic beads (CD34⁺ selection kit. Miltenyi Biotec Inc., Auburn, Calif., USA). CD34⁺ cell purity was >90% by flow cytometry. Cells were transplanted into newborn mice irradiated at 1 Gy using a RS-2000×-Ray Irradiator (Rad Source Technologies, Buford, Ga.). Cells were transplanted by intrahepatic (i.h.) injection of 50,000 cells/mouse in 20 μl phosphate-buffered saline (PBS) with a 30-gauge needle, Human fetal liver cells were isolated from a single donor and cord blood-derived HSC were obtained from two donors. Mice from a single donor were used for all dual treatment mice. Humanization of the animals was affirmed by flow cytometry for the presence of human CD45 and CD3 positive blood immune cells. At 18 weeks of age, 25 NSG-hu mice were infected intraperitoneally (i.p.) with HIV-1_{LN4-3} at 10⁴ tissue culture infective dose₅₀ (TCID₅₀)/ml and sacrificed at days 1, 3, 7, and 14; n=5 at each time point. Five control-uninfected animals were included in all test evaluations. Levels of viral RNA copies/ml were analyzed with the automated COBAS Ampliprep System V2.0/Taqman-48 system (Roche Molecular Diagnostics, Basel, Switzerland). For this assay, 100 μl of mouse serum was diluted to 1 ml with sterile filtered normal human serum. The detection limit of the assay after dilution is 200 viral RNA copies/ml. Although the eclipse phase for viral infection in humans remains variable6l, the viral loads and CD4⁺ T cell depletion levels observed in our infected humanized mice are in point of fact reflective of the disease course in an infected human host. Indeed, only after weeks of infection significant cell loss was observed. These findings can be viewed as an affirmation of the model including CD4⁺ T cell timed-restorations seen after ART as is seen in humans.

Drugs and Antibodies. Dolutegravir (DTG), lamivudine (3TC), and abacavir (ABC) were generous gifts from ViiV Healthcare, Research Triangle Park, NC. Rilpivirine (RPV) was purchased from Hangzhou Bingo Chemical Co., Ltd, Hangzhou, China. Poloxamer 407 (P407), HEPES buffer, ciprofloxacin, paraformaldehyde (PFA), and 3,3′-diaminobenzidine (DAB) were purchased from Sigma-Aldrich, St. Louis, Mo. Diethyl ether, endotoxin-free water, gentamicin, acetonitrile (ACN), methanol, KH₂PO₄, bovine serum albumin (BSA), Triton X-100, LC-MS-grade water, and TRIzol reagent were purchased from Fisher Scientific, Hampton, N.H., USA. FITC-conjugated mouse anti-human CD45, Alexa Fluor-conjugated 700 mouse anti-human CD3, APC-conjugated mouse anti-human CD4, and BV421-conjugated mouse anti-human CD8 antibodies were purchased from BD Biosciences, San Jose, Calif.. Monoclonal mouse anti-human HIV-1p24 (clone Kal-1), monoclonal mouse anti-human leukocyte antigen (HLA-DR; clone CR3/43), and the polymer-based HRP-conjugated anti-mouse EnVision+secondary antibodies were purchased from Dako, Carpinteria, Calif.

For flow cytometric analysis, a panel of antibodies (all from BD Biosciences, San Jose, Calif.) were used and comprised of FITC-conjugated mouse anti-human CD45 (catalog #555482), Alexa Fluor 700-conjugated mouse anti-human CD3 (catalog 4557943), APC-conjugated mouse anti-human CD4 (catalog #555349), and BV421-conjugated mouse anti-human CD8 (catalog #562428), PE-conjugated mouse anti-human CD14 (catalog #555398), and PE-Cy5-conjugated mouse anti-human CD19 (catalog #555414) antibodies. For immunohistochemical staining, monoclonal mouse anti-human HIV-1p24 (clone Kal-1, M0857, Dako, 1:10), monoclonal mouse anti-human leukocyte antigen (HLA-DR; clone CR3/43, Dako, 1:100), and the polymer-based HRP-conjugated anti-mouse EnVision+secondary antibodies were purchased from Dako (Carpinteria, Calif.), Peripheral blood was collected from the submandibular vein into ethylenediaminetetraacetic acid (EDTA)-coated tubes or by cardiac puncture at the study end. Blood leukocytes were tested for human pan-CD45, CD3, CD4, CD8, CD14, and CD19 markers as six-color combinations using LSR-H FACS analyzer (BD Biosciences). Antibodies and isotype controls were obtained from BD Pharmingen, San Diego, Calif., and staining was analyzed with a FlowJo (BD Immunocytometry Systems, Mountain View, Calif.). Results were expressed as percentages of total number of gated lymphocytes. The percentages of CD4 and CD8 positive cells were obtained from human CD3⁺ gate (Dash PK, et al. Long-acting nanoformulated antiretroviral therapy elicits potent antiretroviral and neuroprotective responses in HIV-1-infected humanized mice. AIDS. 2012; 26:2135-2144). Absolute counts of human CD45⁺ cells to normalize each of the human cell data sets. Equivalent numbers of total blood cells/mouse were used at each time point.

GFP expression in infected cells was quantified using Guava EasyCyte Mini flow cytometer (Guava Technologies, Hayward, Calif., USA). Cells were first fixed for 10 minutes in 2% paraformaldehyde then washed 3 times in PBS and analyzed. Peripheral blood was collected from the submandibular vein into ethylenediaminetetraacetic acid (EDTA)-coated tubes or by cardiac puncture at the study end. Blood leukocytes were tested for human pan-CD45, CD3, CD4, CD8, CD14, and CD19 markers as six-color combinations using LSR-II FACS analyzer (BD Biosciences). Antibodies and isotype controls were obtained from BD Pharmingen, San Diego, Calif., USA, and staining was analyzed with a FlowJo (BD Immunocytometry Systems, Mountain View, Calif., USA). Results were expressed as percentages of total number of gated lymphocytes. The percentages of CD4 and CD8 positive cells were obtained from human CD3⁺ gate set (10).

In vivo HIV-1 infection. At 18 weeks of age, 25 humanized NSG (NSG-hu) mice were infected intraperitoneally (i.p.) with HIV-1_{NL4-3} (27, 28) at 10⁵ tissue culture infective dose₅₀ (TCID₅₀)/ml and sacrificed at days 1, 3, 7 and 14; n=5 at each time point. Five control-uninfected animals were included in all test evaluations. Levels of viral RNA copies/ml were analyzed with the automated COBAS Ampliprep System V2.0/Taqman-48 system (Roche Molecular Diagnostics, Basel, Switzerland) (17, 29). For this assay, 100 μl of mouse serum was diluted to 1 ml with sterile filtered normal human serum. The detection limit of the assay after dilution is 200 viral RNA copies/ml.

Immunohistochemistry (IHC) Examinations. Spleen, lung, liver, and lymph nodes were perfused with PBS followed by 4% paraformaldehyde and then post fixed overnight and embedded in paraffin. Five-micron thick sections were cut from the paraffin blocks, mounted on glass slides and labeled with mouse monoclonal antibodies (DakoCytomation, Carpinteria, Calif., USA) for HLA-DQ/DP/DR (clone CR3/43, 1:100) and HIV-1p24 (1:10). The polymer-based HRP-conjugated anti-mouse Dako EnVision system was used as a secondary detection reagent and developed with 3,3′-diaminobenzidine (DAB). All paraffin-embedded sections were counterstained with Mayer's hematoxylin. Deletion of primary antibodies or mouse IgG served as controls. Images were obtained with a Nikon DS-Fi1 camera fixed to a Nikon Eclipse E800 (Nikon Instruments, Melville, N.Y.) using NIS-Elements F 3.0 software.

Generation and pharmacokinetic (PK) testing of LASER ART. LASER ART facilitates sustained inhibition of viral replication by long-acting hydrophobic lipophilic anti-retroviral nanoparticles. To accomplish this goal, fatty-acid-modified prodrugs were synthesized as prodrugs for dolutegravir (DTG), lamivudine (3TC) and abacavir (ABC) by esterification with myristic acid. The chemical structures and physicochemical properties were characterized by nuclear magnetic resonance spectroscopy and Fourier-transform infrared spectroscopy, electrospray ionization mass spectrometry and powder X-ray dif-fraction (Horwitz J. A., et al. Proc. Natl Acad. Sci. USA. 2013;110:16538-16543. Sillman B., et al. Nat. Commun. 2018;9:443. Guo D, et al. J. Acquir Immune Defic. Syndr. 2017;7. Singh D., et al. Nanomedicine. 2016;11:1913-1927. Edagwa B. J., Zhou T, McMillan J M, Liu X M, Gendelman H. E. Development of HIV reservoir targeted long acting nanoformulated antiretroviral therapies. Curr. Med. Chem. 2014;21:4186-4198). The LASER ART particles were characterized fully for stability, size, and shape. This included human monocyte-derived macrophage (MDM) nanoparticle drug uptake, release and potency. Data sets were obtained for nanoformulated myr-istoylated NM (NMDTG), NM3TC and NMABC prodrugs and nanoformulated rilpivirine (NRPV) (Table 8) before being used in the animal studies. These included individual antiretroviral activity for each of the nanoformulations. Moreover, complete PK profiles were performed for each of the nanoformulated drugs after a single drug nanoformulation injection. These are illustrated with the accompanying dosages administered in BALB/c mice (Table 8). The PK measurements including terminal rate constant (λz) and half-life (t_{1/2}), area under the concentration-time curve (AUC), apparent volume of distribution (Vb/F), total plasma clearance of drug (CL/F), mean resident time (of the unchanged drug in the systemic circulation) (MRT), were outlined in prior works (Hunsucker S A, et al. Pharmacol. Ther. 2005; 107:1-30. Yuen G J, Weller S, Pakes G E. Clin. Pharmacokinet. 2008; 47:351-371. Guo D, et al. J. Acquir Immune Defic. Syndr. 2017; 7. Singh D., et al. Nanomedicine. 2016; 11:1913-1927. Kobayashi M, et al. Antimicrob. Agents Chemother. 2011; 55:813-821. Pino S, et al. Methods Mol. Biol. 2010; 602:105-117). These data sets showed tight control over viral replication, and the short tail of drug removal from blood and tissue affirmed that any lack of viral rebound would accurately reflect residual HIV-1 growth rather than any residual antiretroviral drug present as part of the long-acting regimen.

Preparation of Antiretroviral Nanoformulations. Antiretroviral prodrugs and their polymer encasements were performed as previously described (7, 8). Myristoylated modifications for DTC, 3TC, and ABC were made (referred to as MDTG. M3TC, and MARC) to enhance the incorporation into poloxamer 407 (P407) nanoparticles, while RPV was encased solely by poloxamer 338 (P338) in unmodified form using high pressure homogenization to form crystalline nanoformulated drugs. Particle size, polydispersity index, and zeta potential were determined by dynamic light scattering using a Malvern Nano-ZS (Malvern, Worcestershire, UK) (30). Final drug concentrations in the nanoformulation suspensions and injection solutions were determined by HPLC-UV/Vis and UPLC-MS/MS. A 40-50 μl volume for each nanoformulation combination (NMDTG/NRPV and NM3TC/NMABC) was administered by intramuscular (IM) injection in opposing thigh muscles of the mice.

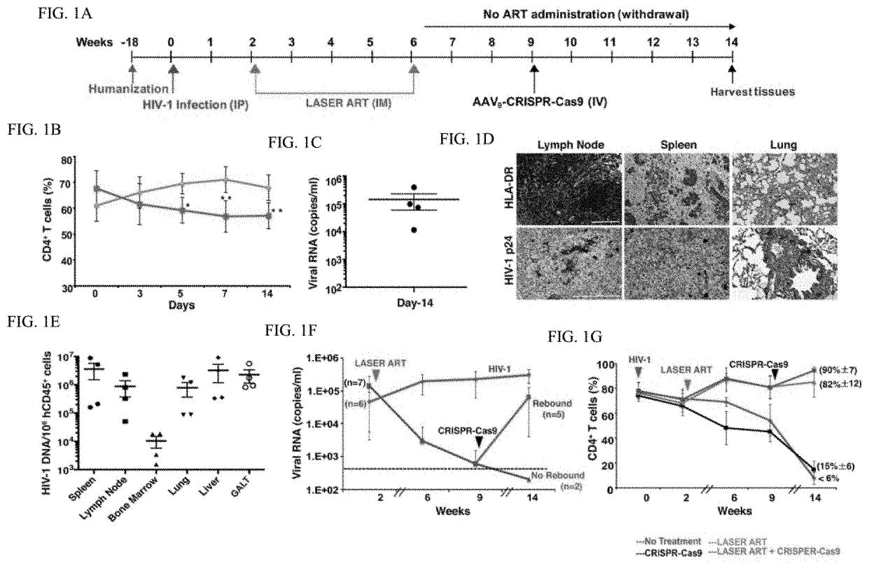

Nucleic Acid Extractions and q-PCR assays. In studies presented in FIGS. 1A-1G, 3A-3E, and 4A-4G total viral nucleic acids (RNA and DNA) extracted from tissue or cells were acquired from the spleen, bone marrow, lung, GALT, liver, kidney, and brain using a Qiagen Kit (Qiagen, Hilden, Germany) according to the manufacturer's instructions. Total cellular DNA and RNA obtained from the HIV-1 infected cell line ACH2 served as a positive control, while human genomic DNA was obtained from uninfected humanized mice as negative controls. Cell-associated HIV-1 RNA and DNA were quantified by q-PCR and droplet digital PCR (ddPCR) assays. Because of extremely low numbers of latently-infected human cells in HIV-infected humanized mice after long-term ART, detection of total HIV-1 DNA, requires two rounds of PCR amplification. The first round of PCR was performed on a conventional PCR machine (T100 Thermal Cycler, Biorad, Calif., USA) in 25 μl of PCR master mix containing 5 μl of template and 50 ng each of both primers annealing to HIV gag region as follows: 94° C. for 3 min, followed by 15 cycles of 94° C. for 30 s, 55° C. for 30 s, and 72° C. for 1 min. The product of the first PCR was subsequently used as a template in the second semi-nested real-time PCR amplification performed on the ABI Prism 7000 real-time PCR machine (Applied Biosystems, Mass., USA) using TaqMan detection probe. A total of 2 μl of the first PCR product was diluted to 50 μl with PCR master mix containing 0.2 μM concentrations each of both primers and 0.2 μM TaqMan dual-labeled fluorescent probe. Real-time PCR settings were as follows: 50° C. for 2 min, then 95° C. for 10 min, followed by 40 cycles of 95° C. for 15 s and 60° C. for 1 min. The amplicon sizes are 221 bp for the first round of PCR and 83 bp for the second round (real-time) PCR. ACH2 cells (8×10⁵) containing one integrated copy of HIV-1 per cell were used in triplicate as standards and HIV copy numbers ranging in serial 10-fold dilutions from 10¹ to 10⁵ DNA copies/reaction (17, 18). Integrated DNA provirus was quantified using an adapted alu-PCR assay as described by Agosto et al. (31) with modifications for the second round of PCR, following prior published methods (32). Briefly, samples underwent a first-round PCR amplification (95° C. for 2 min; 20 cycles of 95° C. for 15 s, 50° C. for 15 s, and 72° C. for 150 s) using 100 nM alu and 600 nM gag reverse primers. Five μl of the first-round product were amplified in a nested protocol using the assay for HIV-1 gag gene (second PCR primers and probe), as described above. A first-round PCR with 3 replicates using only the gag reverse primer (gag only) acted as background un-integrated control. Serially diluted integration site standards were used to construct a standard curve for each plate. Integration levels per cell were calculated by subtracting gag-only signals from the alu-gag quantification. Semi-nested real-time PCR on HIV-1 RNA was performed as described (17, 18). The eluted cellular RNA was first subjected to DNase treatment to remove HIV-1 DNA to avoid the interference with the quantitation. For reverse transcription assay, random hexamers were used as primers and SuperScript III (Invitrogen, Mass., USA) to synthesize first-strand cDNA at 42° C. for 60 min. cDNA was used for the unspliced (usRNA) assay. Two rounds of PCR were performed under the same PCR conditions as described for total viral DNA. For the usRNA assay, real-time PCR was run for 45 cycles and same primers and fluorescent probe as for the total viral DNA assay were used. Human CD45 species-specific primers and probes were obtained from Thermo-Fisher Scientific (USA) (cat. no. 433182 for Hs0036534_g1).

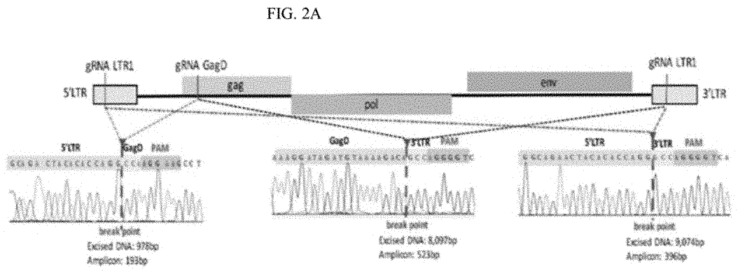

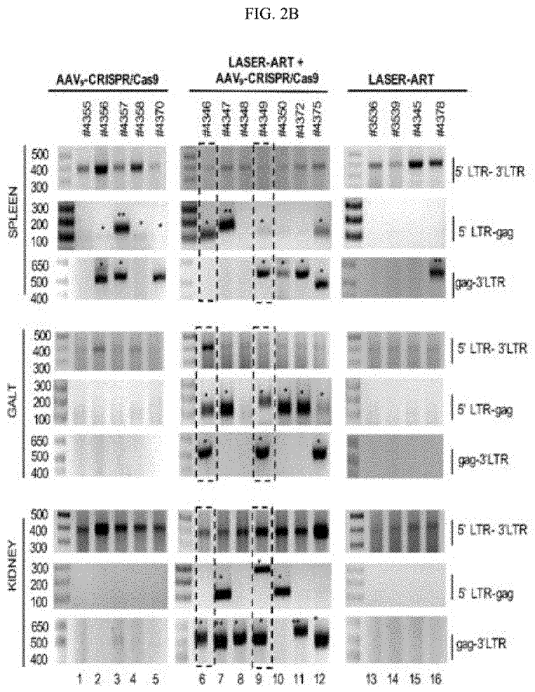

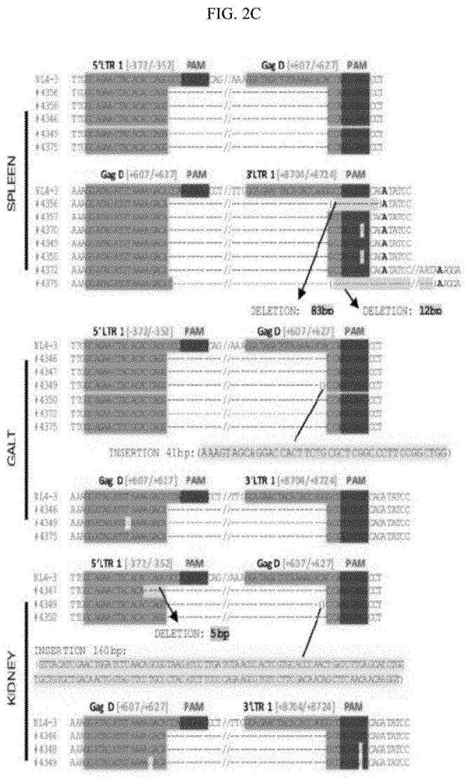

In studies presented in FIGS. 2A-2C, 11, 12A-12C, 13A-13M, 14A-14F, 20A-20D, 21A, 21B and 22A, 22B and, frozen tissues were homogenized using Bullet Blender homogenizer (Next Advance, Averill Park, N.Y., USA) using bead combinations and settings specific for every tissue according to manufacturer protocols. T1 buffer from NucleoSpin Tissue kit (Macherey-Nagel, Duren, Germany) was used for homogenization/initial lysis followed by over-night proteinase K digestion. Extraction of genomic DNA was completed according to the protocol of the manufacturer. For standard PCRs (Table 1.1), 500 ng of extracted DNA were subjected to PCR using Fail Safe PCR kit and buffer D (Epicentre, Madison, Wis., USA) under the following PCR conditions: 94° C. 5 minutes, 30 cycles (94° C. 30 s, 55° C. 30 s, 72° C. 30 s), 72° C. 7 minutes using 1st round primers followed by nested PCR using diluted 1st round PCR reaction. Nested PCR products were subjected to Sanger sequencing directly if only one amplicon population was detected by agarose gel electrophoresis. For multiple amplicons detected, or to investigate the composition of HIV excision, each amplicon population was separated and purified from an agarose gel electrophoresis and then cloned into TA vector (Invitrogen, Carlsbad, Calif., USA). Plasmid DNA containing excised HIV amplicon was purified from each bacterial colony for Sanger sequencing (Genewiz, South Plainfield, N.J., USA). HIV-1 DNA was quantified using TaqMan qPCR specific for HIV-1 pol and env genes and cellular beta-globin gene as a reference (Table 1.2). Prior to qPCR, genomic DNA was diluted to 10 ng/ul and then 5 μl (=50 ng) were taken per reaction/well. Reaction mixtures were prepared using Platinum Taq DNA Polymerase (Invitrogen) according to a simplified procedure (33) Standard was prepared from serial dilutions of U1 cells genomic DNA since it contains two single copies of HIV-1 provirus per diploid genome equal to beta-globin gene copy number. qPCR conditions: 98° C. 5 minutes, 45 cycles (98° C. 5 minutes, 45 cycles (98° C. 15 s, 60° C. 30 s with acquisition, 72° C. 1 minute). Reactions were carried out and data analyzed in a LightCycler96 (Roche, Basel, Switzerland). For RT PCR, TRIzol reagent (Ambion, Austin, Tex., USA) was used for initial RNA extraction followed by clean up using RNeasy kit (Qiagen, Hilden, Germany) with DNAse I digestion in the extraction column. Total 0.5 μg of RNA was used for M-MLV reverse transcription (Invitrogen). For gRNA expression screening specific reverse primer (pX601gRNA scaffold/R, Table 1.3.) was used in RT reaction followed by standard PCR using target LTR 1 or Gag D sense oligos as forward primers (Table 1.3) and agarose gel electrophoresis. For checking saCas9 mRNA expression oligo-dT primer mix was used in RT and cDNA was subjected to PCR using saCas9 specific primer pairs and β-actin as a reference (Table 1.3). Sanger sequencing results were analyzed using Clustal Omega (EMBL-EBI) multiple sequence alignment tool and Sequence Scanner Software 2 (Applied Biosystems).

Off-Target Analysis—Cell Culture Model: TZM-bl cells were plated in 6 well plates at 1×10⁵ cells/well and co-transfected using Lipofectamine 2000 reagent (Invitrogen) with 1 μg of control pX601-AAV-CMV:NLS-SaCas9-NLS-3xHA-bGHpA; U6::Bsa1-sgRNA (Addgene #61591) or 1 μg of pX601-LTR1-GagD (16) plasmid together with 0.2 μg of pKLV-U6gRNA(Bbs1)-PGKpuro2ABFP (Addgene 50946) to provide puromycin selection marker. Next day, cells were transferred into 100-mm dishes and cultured in the presence of puromycin (Sigma) at concentration 1 μg/ml. After two weeks, surviving clones were isolated using cloning cylinders (Corning, Corning, N.Y., USA). Genomic DNA was prepared from each single cell clone and LTR specific PCRs followed by gel purification; TA cloning and Sanger sequencing were performed. The clones showing the presence of on target CRISPR-Cas9 induced InDel mutations at target LTR 1 site in integrated HIV-1 LTR sequence (n=6) together with two control clones were selected for further in vitro off target analysis. The list of potential OFF target sites in human genome for HIV-1 target LTR 1 and Gag D was created using Benchling CRISPR design tool (Benchling, San Francisco, Calif. 94103 (benchling.com) Tables 2 and 3). Total three potential OFF target sites were chosen (the top scorer plus two top gene specific potential off target sites, see Tables 2 and 3, highlighted in yellow) for PCR based screening in selected single cell clones. The potential OFF target regions were PCR amplified, cloned into TA vector, and sent for Sanger sequencing (3-6 sequences/single cell clone/single OFF target).

The genetic variation analyses among the three treatments were performed through the next generation sequencing (by the Novogene NGS facility) and bioinformatics tools for four sample animals, one animal from the LASER ART, one animal from CRISPR-Cas9- and two no-rebound animals from the LASER ART/CRISPR-Cas9 groups. The main objective was to detect the possible CRISPR-Cas9 off-target sites. Besides this, some genetic variations such as single nucleotide polymorphisms (SNP), insertion-deletions (InDels), structural variants (SVs), and copy number variants (CNVs) were analyzed for those four animals. After a thorough quality control step, the resulting paired-end short-reads were mapped to the human reference genome (Human_G1K_V37) utilizing Burrows-Wheeler Aligner (BWA) algorithm. For the animals M4356 (CRISPR-Cas9), M4348 and M4349 (LASER ART+CRISPR-Cas9), and M3539 (LASER ART), the 8 coverages were reported to he 92.01%, 91.97%, 92.01%, and 91.92%, while the sequencing depths were 36.08, 63.11, 45.22, and 15.41, respectively.

Viral Recovery. PBMCs obtained from leukopaks from HIV-1,2 seronegative donors were stimulated with PHA and IL-2 and co-cultured with human bone marrow or spleen cells recovered from 3 groups of CD34⁺ HSC-NSG mice that included HIV-1 infected, infected and LASER ART treated, and LASER ART and AAV₉-CRISPR-Cas9-treated mice. PBMCs were used in assays after a 3-day treatment maintained in. 10% RPMI with 30 U/ml of IL-2 then co-cultured with human bone marrow or spleen cells at concentrations of (1:5). Cells were harvested eight days later for HIV-1 DNA (A) and RNA (B) using semi-nested real-time qPCR assay and supernatant fluids assayed for reverse transcriptase activity for up to day-14. Data are expressed as total HIV-1 DNA (A) or RNA (B) copies/10⁶human CD45⁺ cells. One of the two dual-treated animals was tested and confirmed viral sterilization. Viral rescue was observed in other groups of animals tested.

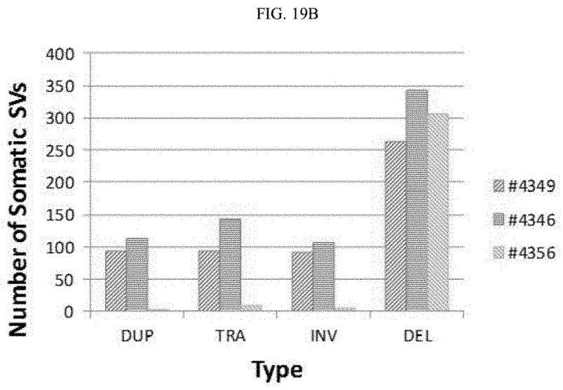

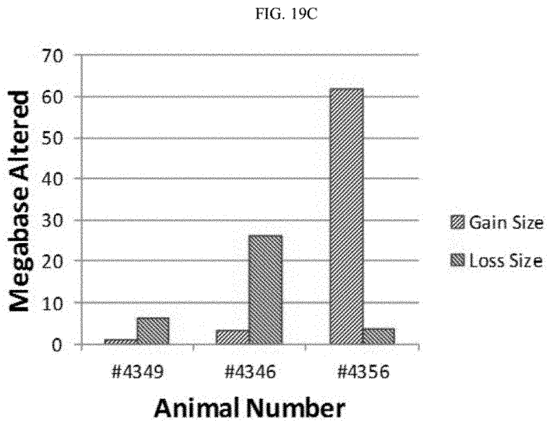

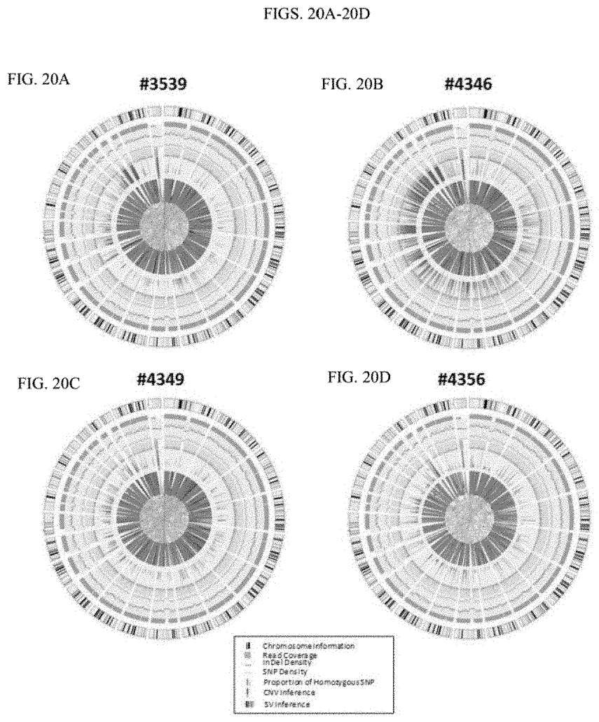

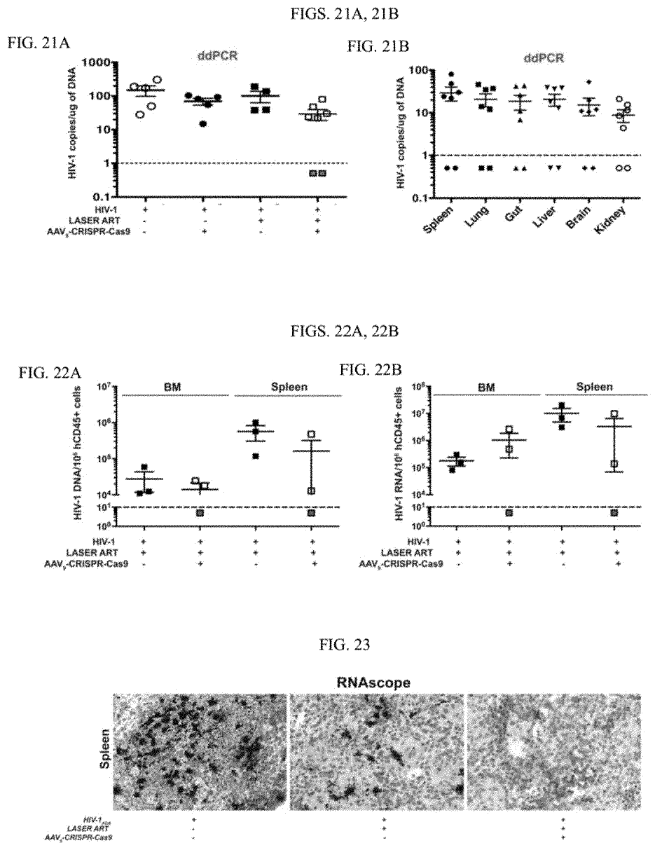

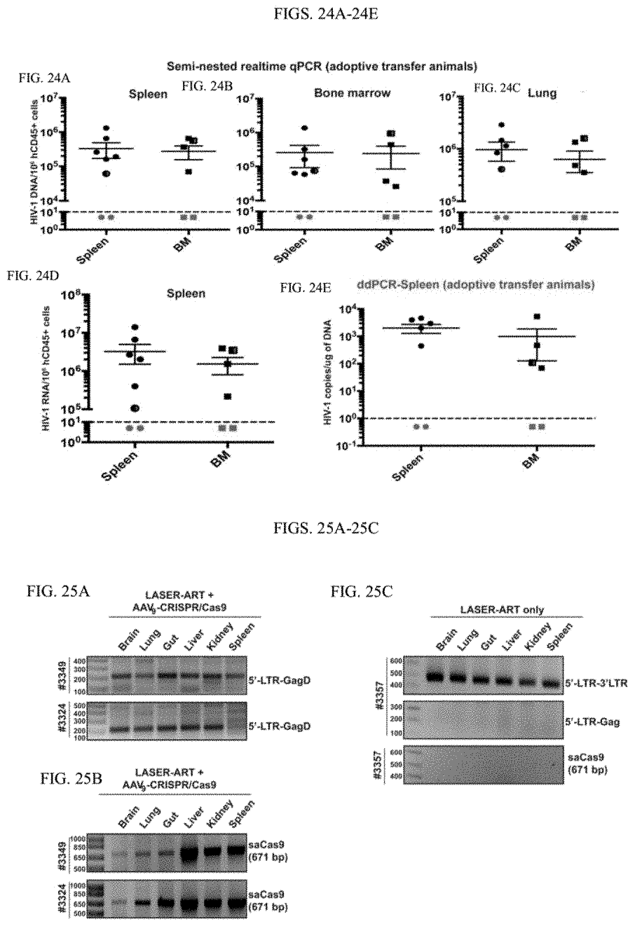

Humanized Mice Model. The genetic variation analyses among the three treatments were performed through the next generation sequencing (by the Novogene NGS facility) and bioinformatics tools for four sample animals, one animal from the LASER ART, one animal from CRISPR-Cas9- and two no-rebound animals from the LASER ART+CRISPR-Cas9 groups. The main objective was detecting the possible CRISPR/Cas9 off-target sites. Besides this, some genetic variations such as single nucleotide polymorphisms (SNP), -nsertion-deletions (InDels), structural variants (SVs) and copy number variants (CNVs) were analyzed for those four animals and the results are given in FIGS. 19A-19C, 20A-20D and Tables 4-6. After a thorough quality control step, the resulting paired-end short-reads were mapped to the human reference genome (Human_G1K_V37) utilizing Burrows-Wheeler Aligner (BWA) algorithm. For the animals #4356 (CRISPR-Cas9), #4348 and #4349 (LASER ART+CRISPR-Cas9) and #3539 (LASER ART), the coverages were reported to be 92.01%, 91.97%, 92.01% and 91.92%, while the sequencing depths were 36.08, 63.11, 45.22, and 15.41, respectively.

Adoptive Transfers. Splenocytes and bone marrow cells (8-10×10⁶) were harvested at the time of sacrifice from NSG-hu mice that were HIV-1_{ADA} infected with and without LASER ART and AAV₉-CRISPR-Cas9. The cells were adoptively transferred into unmanipulated 18-week old CD34 HSC-NSG mice. Cell counts and viability tests were determined by both trypan blue and live/dead stains on the TC-20 automated cell counter (Bio-Rad). Cells were injected IP into mice and monitored for an additional 4 weeks. These experiments were performed to cross validate eradication of viral infection that could occur from latent reservoirs and not detected by either qPCR, RNAscope, and ddPCR assays. Viral load was measured from blood samples of the adoptively transferred mice using automated. COBAS Ampliprep System V2.0/Taqman-48 system, and immune cell profiles (CD4 and CD8⁺ T cells by flow cytometry) recorded, in parallel. Residual virus from all humanized mice tissues was examined by qPCR and ddPCR assays. Virus was not detected in plasma or tissues from two adoptively transferred animals (mice M3319 and M3336).

ddPCR for Detection of HIV-1 Nucleic Acids. ddPCR was performed based on the water-oil emulsion droplet technology and used for viral detection using the outlined primers (Forward-5′-TCAGCCCAGAAGTAATACCCATGT-3′ (SEQ ID NO: 46) and Reverse-5′-CACTGTGTTTAGC ATGGTGTTT-3′ (SEQ ID NO: 47)) and a TaqMan probe. The ddPCR assay was run with the ddPCR™ SUPERMIX for Probes reagents in the QX200™ DROPLET DIGITAL™ PCR system (Bio-Rad Laboratories, Hercules, Calif., USA). For quantification of integrated HIV-1 DNA, the eluted cellular DNA was PCR amplified (17, 18, 31, 32) for integrated viral DNA (iDNA) targeting the HIV-1 gag gene. Total 100 ng of each tissue DNA template were used for ddPCR amplifications and performed on the QX200™ DROPLET DIGITAL™ PCR system (Bio-Rad Laboratories, Hercules, Calif., USA) using the ddPCR™ Supermix for Probes reagents following the thermal cycling conditions for TaqMan detection. Data acquisition and analysis were done using QX200 droplet reader and QUANTASOFT™ software (Bio-Rad Laboratories, Hercules, Calif., USA).

RNAscope Assay. Viral RNA was detected as single brown dots or cluster of dots in 10 μm thick spleen tissue sections using antisense probe V-HIV1-Clade-B (ACD cat no 416111) targeting 854-8291 bp of HIV-1_{NL4-3} (34) Human peptidylprolyl Isomerase B (PPIB) was used as positive control for the spleen tissue analyzed (images were captured at 40× magnification).

Viral Recovery. Phytohemaglutinin (PHA) and interleukin-2 (IL-2) stimulated peripheral blood mononuclear cells (PBMCs) obtained from leukopaks from HIV-1,2 seronegative donors were co-cultured with human bone marrow (BM) or spleen cells recovered from infected and or LASER ART with and without AAV₉-CRISPR-Cas9 treated humanized mice. PBMCs were used in assays after 3-day treatment maintained in 10% RPMI with 30 U/ml of IL-2 then co-cultured with human BM or spleen cells at concentrations of (1:5) (35-37). Cells were harvested eight days later for HIV-1 DNA (A) and RNA (B) using semi-nested real-time PCR assay and supernatant fluids assayed for reverse transcriptase activity. Data are expressed as total HIV-1 DNA (A) or RNA (A) copies/10⁶ human CD45 cells. One of two dual treated animals was tested and confirmed viral sterilization. Viral rescue was observed in other animals tested.

Excision Efficiencies and Hierarchal Clustering: The excision efficiencies for each animal, tissue, and HIV-1 gene segment were calculated as the ratio of the number of the sequencing-verified PCR product to all members in each group with denoted experimental conditions (i.e. treatments, tissues, etc. shown in FIGS. 2A-2C, 11, 12A-12C, 13A-13M). Defined in such a way that the excision efficiencies can be viewed as frequentist probabilities, i.e. the ratio of the frequency of occurrence of the event of interest to the total number of experimental repeats. This interpretation of excision efficiencies provides the user with a predictive value, as they can be used to set a prior expectation on the success rate of each treatment (LASER ART, CRISPR-Cas9, and LASER ART plus CRISPR-Cas9) in excising the desired segments of HIV-1 gene in the studied tissues and further to relate that to the likelihood of cure.

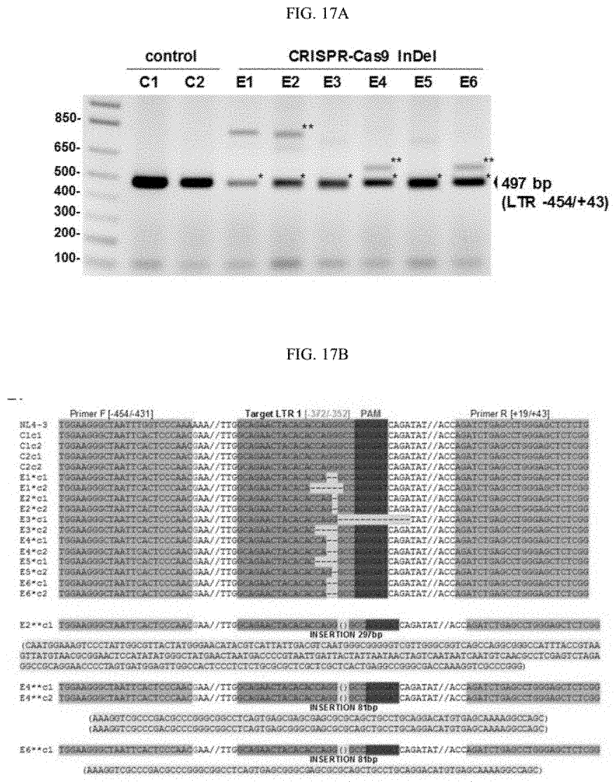

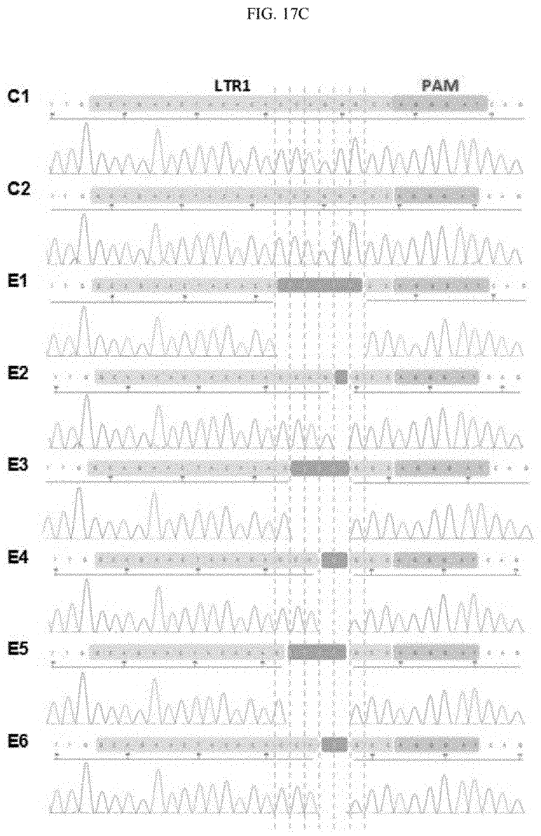

Hierarchical clustering was performed on the efficiency values of truncation events under different treatments and across different animals, tissues, and HIV-1 gene segments. Once the excision efficiencies were calculated under different combinations of experimental conditions, the hierarchical clustering scheme was employed to group the efficiency values into a multilevel cluster tree represented by a dendrogram. The corresponding efficiency values were listed in heat-map table, to make the clusters visually detectable. To this end, three combinations were considered: i) excision probabilities of different HIV-1 segments in 6 different tissues of animals undergoing antiretroviral treatment, CRISPR-Cas9 mediated editing, and the combined treatments (FIG. 17A); ii) excision probabilities of different segments in different animals under the three treatments (FIG. 17B); and iii) probabilities of observing at least one positive band for each specified tissue in all animals (FIG. 17A). Clusters of FIGS. 17B and 17C also include additional conditions of “Cure” and qPCR data to identify which animals experienced complete cure and highest viral genome eradication. Note that, in all figures, S1 refers to 5′ LTR to Gag excision and S2 refers to Gag to 3′ LTR excision of the HIV-1 genome, respectively.

Study Approval. All experimental protocols involving the use of laboratory animals were approved by the University of Nebraska Medical Center (UNMC) Institutional Animal Care and Use Committee (IACUC) ensuring the ethical care and use of laboratory animals in experimental research. Human blood cells were isolated by leukapheresis from HIV-1/2 and hepatitis seronegative donors and were deemed exempt from approval by the Institutional Review Board (IRB) of UNMC. Human CD34⁺ hematopoietic stem cells were isolated from human fetal liver and umbilical cord blood and are exempt from UNMC IRB approval.

Statistics. The data were analyzed using GraphPad Prism 7.0 software

Statistical analyses. Data were analyzed by GraphPad Prism 7.0 software (La Jolla, Calif., USA). Data are represented as the mean±the standard error of the mean (SEM). Experiments were performed using a minimum of three biologically distinct replicates. For comparisons of two groups, Student's t test was used. T cell populations, viral RNA and DNA, and viral load were analyzed by one-way ANOVA with Bonferroni correction for multiple-comparisons. For studies with multiple time points, two-way factorial ANOVA and Bonferroni's post-hoc tests for multiple comparisons were performed. Multiple comparisons were corrected for the false discovery rate (FDR) using the Benjamini-Hochberg procedure. Animal studies included a minimum of 5-7 animals per group unless otherwise noted. Extreme outliers beyond the 99% confidence interval of the mean and 3-fold greater than the SEM were excluded. Significant differences were determined at a p<0.05.

Results

Creation and characterization of HIV-1 infected humanized mice. With the knowledge that few small animal models of HIV-1 reflect actual viral reservoirs and long-term infections, another system for study is required. This is based both on known species restrictions for HIV-1 infection and long-term establishment of tissue reservoirs of infection. Human hematopoietic stem cells (HSC) reconstituted NOD.Cg-Prkd^{scid}Il2rgt^{mlW}j^{l}/S_{z}J (NSG) mice produce human T cells, that are broadly susceptible to HIV-1 infection (White M. K., Hu W., Khalili K. PLoS Pathog. 2016; 12:e1005953. doi: 10.1371/journal.ppat.1005953. Yin C. et al. Mol. Ther. 2017; 25:1168-1186. Hunsucker S. A. et al. Pharmacol. Ther. 2005; 107:1-30. Yuen G. J. et al. Clin. Pharmacokinet. 2008; 47:351-371. Singh H. et al. Curr. Clin. Pharmacol. 2016; 11:88-94. Ford N. et al. HIV/AIDS. 2011; 3:35-44. Zhou T. et al. Biomaterials. 2018; 151:53-65. Gorantla S. et al. J. Virol. 2007; 81:2700-2712). The model permits evaluation of long-term viral infection in blood and tissues and ART-induced HIV-1 latency. To affirm the model's relevance for studies of HIV-1 elimination, a detailed evaluation of each of the human cell-virus model components was undertaken). First, after irradiation of mice at birth, animals were engrafted with human CD34+ HSC isolated from cord blood by a single intrahepatic injection. The presence of human immunocytes in blood was confirmed by flow cytometry. Second, four months after humanization was confirmed animals were infected with HIV-1_{ADA} at 10⁴ tissue culture infection dose₅₀ (TCID₅₀)/animal and analyzed for acute (14 days) and chronic (16 weeks infection) paradigm. At sacrifice, human cell reconstitution was confirmed in tissues (spleen, lymph node, liver, lung and brain) by immunohistochemical staining with human HLA-DR antibodies. Anatomical localizations and lymphocyte prominence were confirmed by human cell penetration into the white and red pulp and germinal centers of spleen. Lymph nodes were enriched with human cells with anatomical distinctions in the cortex, medulla and germinal centers. Third, productive HIV-1 infection was confirmed by HIV-1p24 staining as shown by large numbers of stained cells. Infection was highest in lymphoid compartments as compared to liver, lung and brain. A significant CD4⁺ T-cell decline and increased CD8⁺ T-cell numbers were observed as a consequence of sustained HIV-1 infection. The percentage of human CD4⁺ T cells in mice was determined in blood by flow cytometry at 2, 6, 11, and 15 weeks and showed decline after infection. Plasma viral RNA copies/ml 16 weeks after HIV-1 infection were readily observed.

A functional cure of HIV infection was documented in a single person (1). However, efforts were stalled by a combination of limited therapeutic access to viral reservoirs, rapid spread of infection, high numbers of virus susceptible cells, and complete inability to eliminate latent integrated proviral DNA. These therapeutic treatments have precluded viral eradication as rebound was seen after cessation of antiretroviral therapy (ART) (2-6).

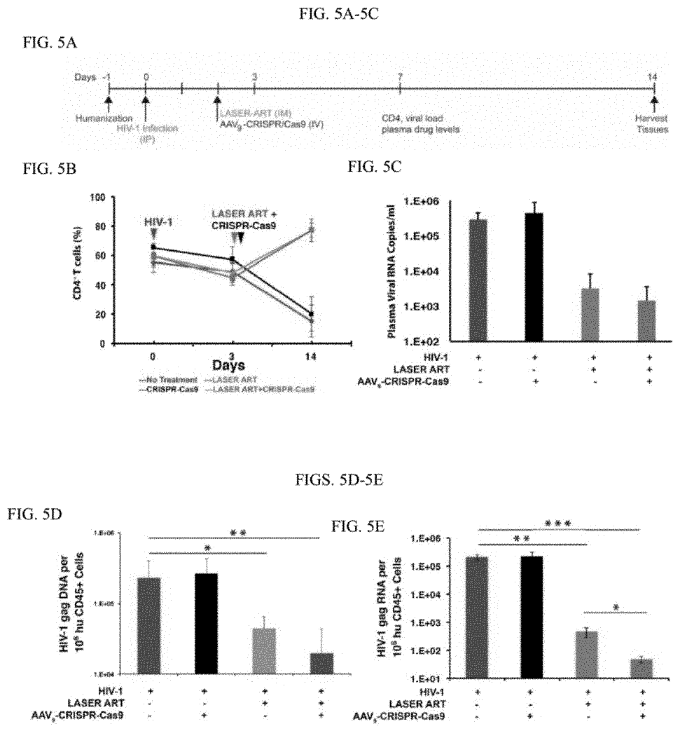

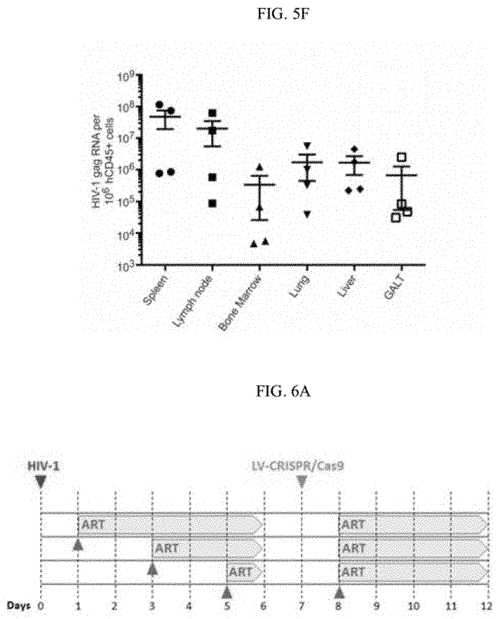

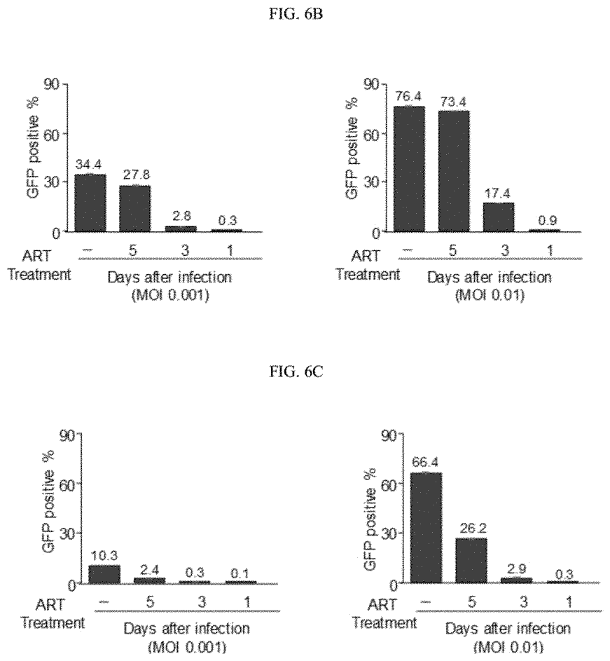

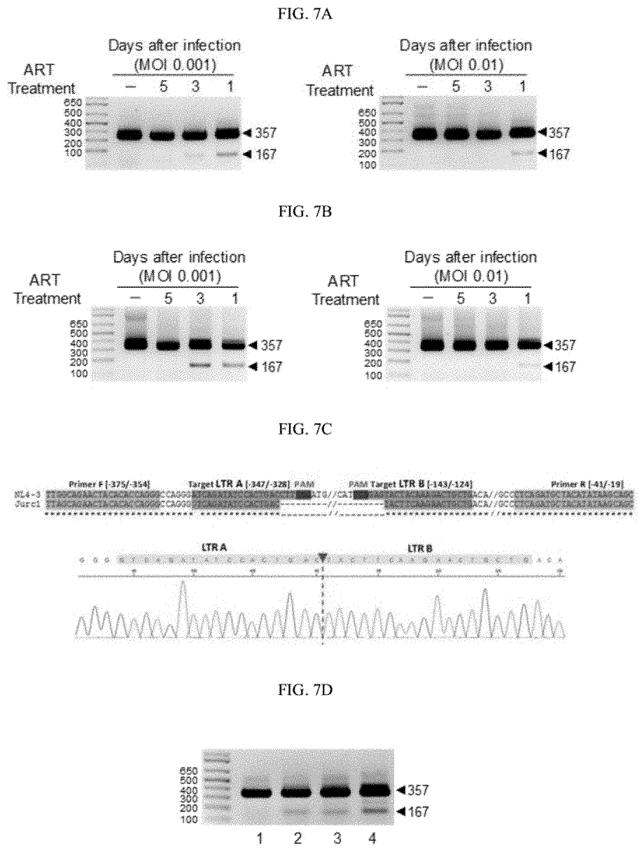

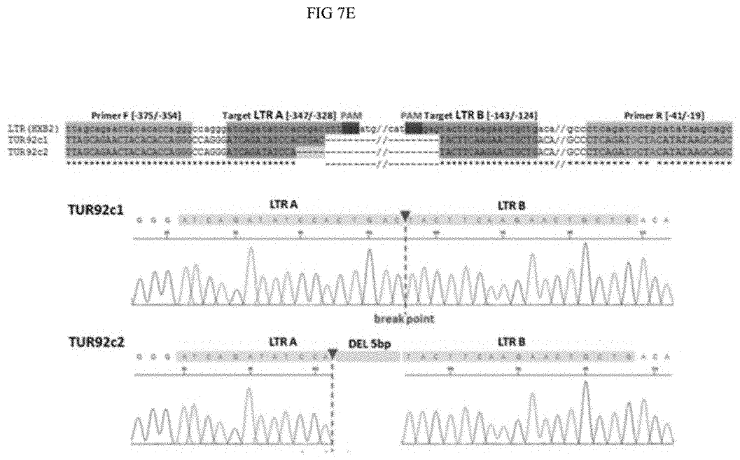

To address each of these limitations, highly hydrophobic and lipophilic antiretroviral prodrugs termed herein “long-acting slow effective release antiretroviral therapy” (LASER ART), were produced to improve drug penetrance across cell and tissue barriers and improve control over ongoing viral infection (7-10). Further, CRISPR-Cas9 technology was employed that specifically and efficiently excised fragments of integrated HIV-1 proviral DNA from the host genome in cell cultures as well as in several tissues from small animal models (11-16). To provide proof of concept that LASER ART and CRISPR-Cas9 treatments could produce synergy towards viral elimination, gut-associated lymphoid tissue (GALT), spleen, lymph nodes, brain, lung, liver, and kidney tissues of NOD.Cg-Prkdc^{scid}Il2rg^{tmlWjl}/SzJ (NSG) mice were populated with human peripheral blood lymphocytes (PBLs) then infected with 10⁴ tissue culture infective dose⁵⁰ (TCID₅₀) of HIV-1_{NL4-3} (17, 18). Three days later, animals were divided into four groups (n=7 for each group). Groups were control (uninfected) and infected animals left untreated or treated with LASER ART as defined by combinations of myristoylated dolutegravir (DTG), lamivudine (3TC) (8) and abacavir (ABC) (7) prodrugs and rilpivirine (RPV) nanoformulations with or without AAV₉-CRISPR-Cas9. All treatments were simultaneously administered. After two weeks, CD4+ T cells and viral DNA and RNA levels were assessed in blood and tissues. No significant differences in the levels of CD4⁺ T cells and viral DNA and RNA levels were observed between the treatment groups. However, animals treated with both LASER ART and AAV₉-CRISPR-Cas9 viral RNA and DNA levels were decreased more than those receiving LASER ART which by itself restored CD4⁺ T cells and reduced plasma viral RNA to or below baseline (FIGS. 5A-5F). These results provide evidence that CRISPR-Cas9 would be most effective in HIV-1 LTR and the Gag gene excision in an ART setting. Support for this notion was realized from follow up in vitro and ex vivo studies. These investigations showed significant increases in the proficiency of CRISPR-Cas9 excision of HIV-1 proviral DNA in infected T-cells following ART-induced viral restriction (FIGS. 6A-6C, 7A-7E).

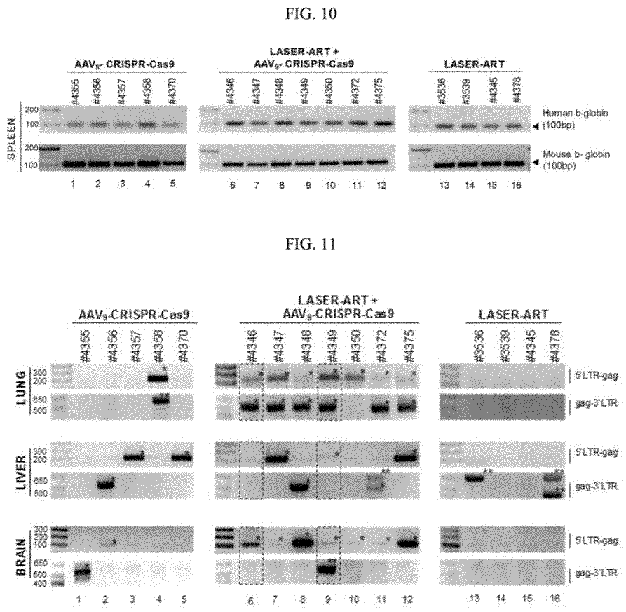

Based on these observations, an amended treatment strategy was adopted (FIG. 1A) based on the assumption that ART-induced viral suppression would improve CRISPR-Cas9 editing efficiency and facilitate viral elimination without bystander tissue toxicities (16, 19, 20) humanization of the animals was confirmed by flow cytometry for CD45 and CD3 staining of blood immune cells in animals demonstrating human cell survival in all mouse groups (FIGS. 8A and 8B). All infected animals showed marked depletion of CD4⁺ T cells (FIGS. 1B and 1G) and plasma viral RNA levels at a median of 2.2×10⁵ copies/ml (FIGS. 1C and 1F). Immunohistochemical examination for HIV-1p24 antigens in spleen, lymph node, and lung showed broad distribution of infected HLA-DR reactive human cells (FIG. 1D). Semi-nested real-time q-PCR HIV-1 nucleic acid detections confirmed viral infection in tissue (FIG. 1E). These infected animals were then divided into four groups. The first received no treatment; group 2 received a single intravenous (IV) injection of AAV₉-CRISPR-Cas9, and the third and fourth groups received 40-45 mg/kg LASER ART. The fourth group, in addition, received AAV₉-CRISPR-Cas9 after three weeks of LASER ART. After the last administration of LASER ART, animals were observed for 8 weeks for any evidence of viral rebound, which corresponded to 5 weeks of AAV₉-CRISPR-Cas9 treatment. Such rebound was observed in all groups with the exception of two animals in group 4 animals #4346 and #4349, which received a combination LASER ART and CRISPR-Cas9 (FIG. 1F). Restoration of CD4⁺ T cell counts (90%±7%) was observed in dual treated group 4 animals, which was higher than those seen in group 3 that received only LASER ART (82%±12%). In the absence of LASER ART, restoration of CD4⁺ T cells with CRISPR-Cas9 treatment (group 2) remained low (15%±6), yet slightly higher than those seen in Group 1 (no treatments, less than 6%) (FIG. 1G). Immunohistochemical evaluation of spleens from HIV-1 infected animals for the presence of CD4+ T cells showed comparable increases (FIG. 9). Further, detection of human DNA sequence in spleen confirmed the presence of the human cells in the spleen of the humanized animals (FIG. 10).

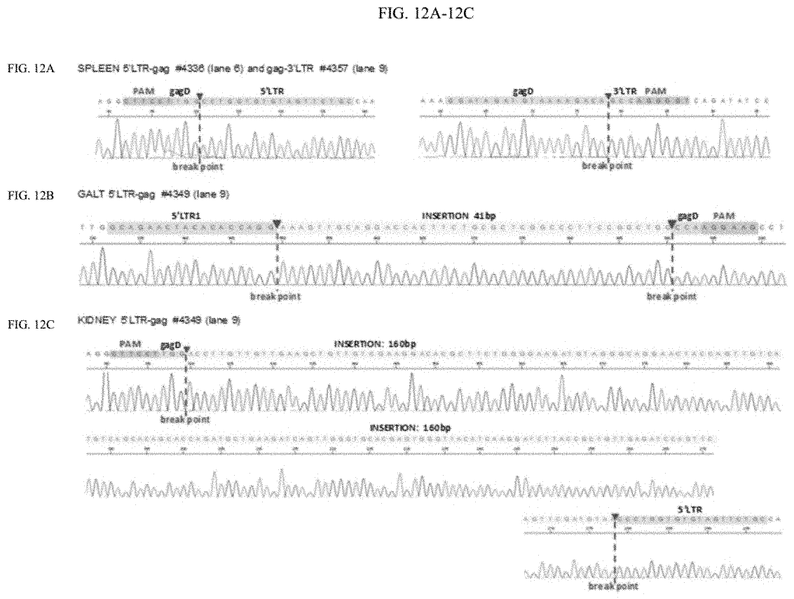

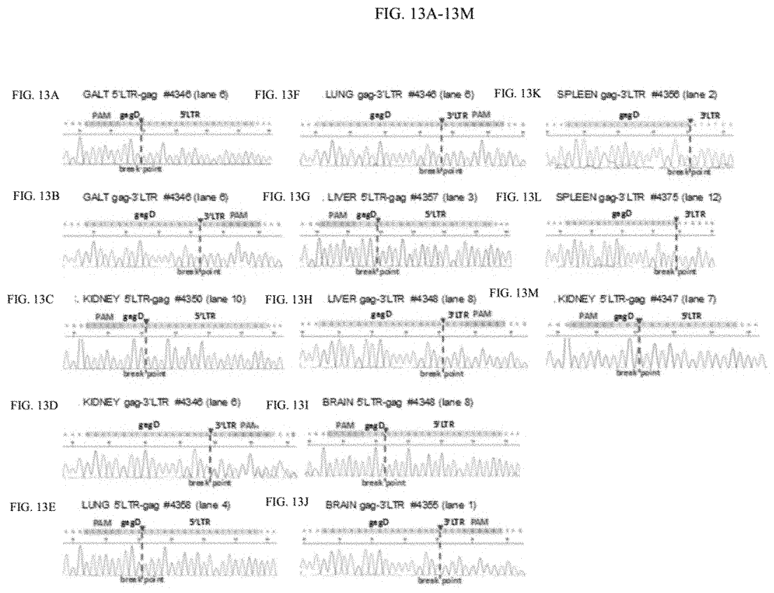

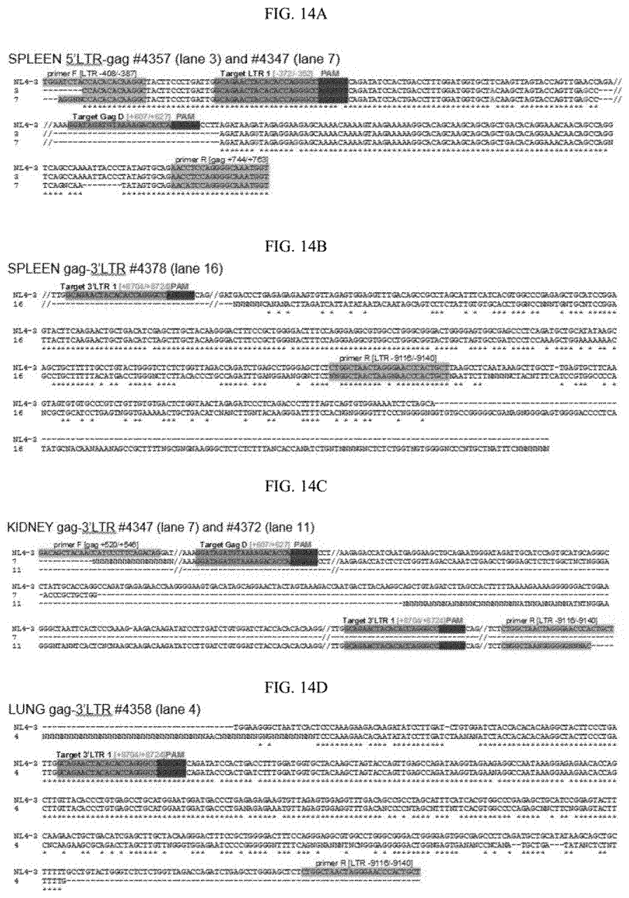

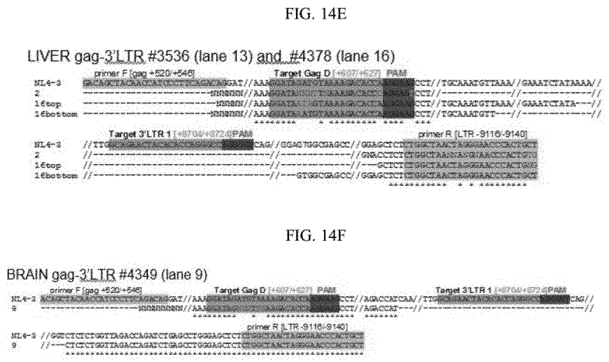

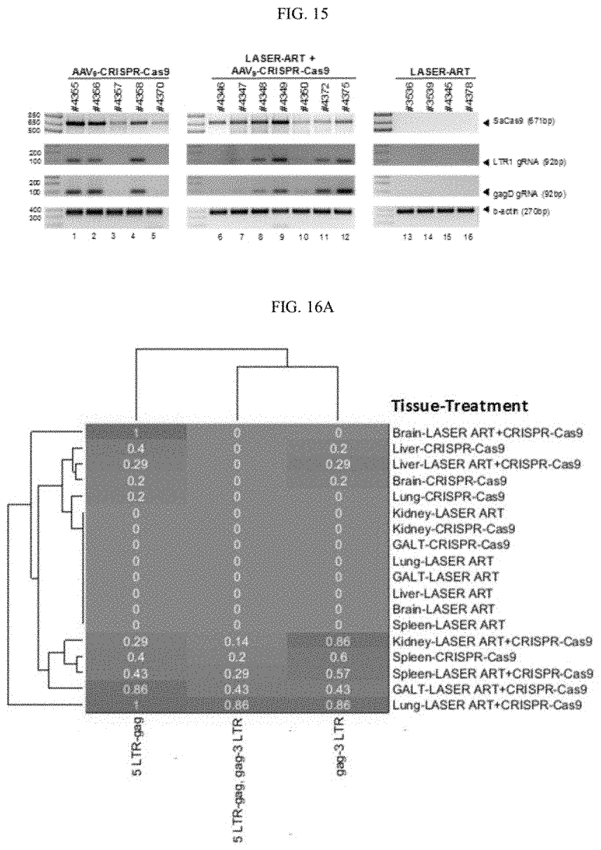

Gel electrophoresis analysis of the PCR amplified DNA fragments using specific pairs of primers designed for detection of the various cleavage events (FIG. 2A) revealed robust cleavage and excision of viral DNA fragments obtained from spleen, GALT, and kidney of the group of animals treated with LASER ART and AAV₉-CRISPR-Cas9 (FIG. 2B). Also, it was noted that the type of excision differed in various tissues among the animals as well as in the same individual animals. Efficient excision of the predicted fragment in other tissues including lung, liver, and brain was also observed in some of the animals with dual treatments (FIG. 11). The integrity and precision of the HIV-1 DNA excision by CRISPR-Cas9 were sequence verified (FIG. 2C, and FIGS. 12A-12C, 13A-13M). In mice that received AAV₉-CRISPR-Cas9 without LASER ART fragmental deletion was detected. Several other DNA fragments in tissues from the experimental animals, including those that received only LASER ART, were amplified, which after sequencing were found unrelated to HIV or editing by CRISPR-Cas9 that may represent replication defective HIV-1 (highlighted by double asterisks) (FIG. 2B and FIGS. 14A-14F). Amplification of the DNA fragments corresponding to control housekeeping actin gene in the various tissues and expression of gRNAs and Cas9 are shown (FIG. 15).

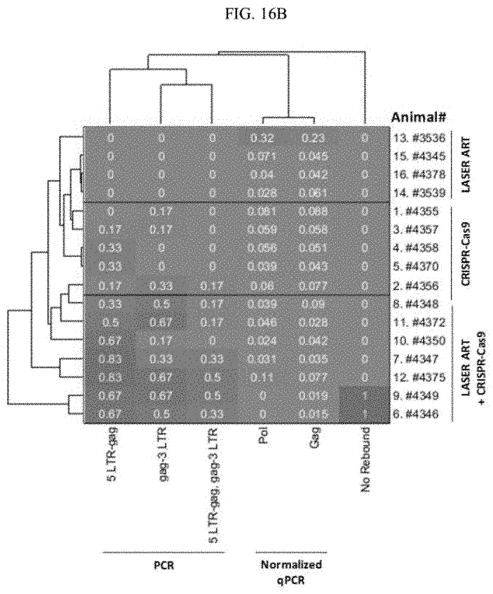

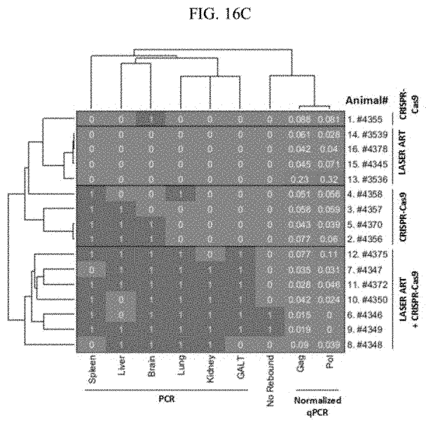

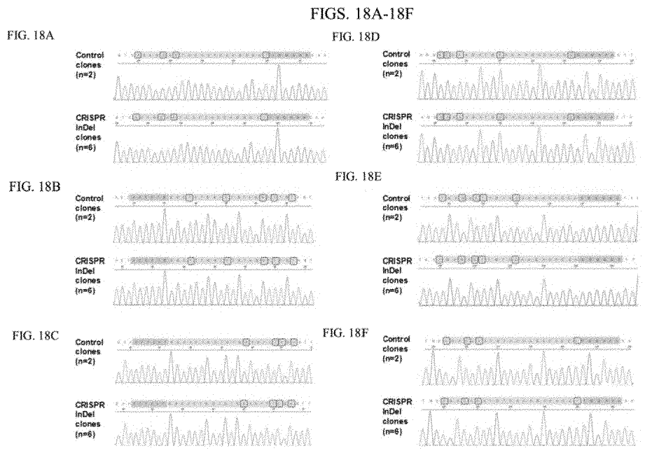

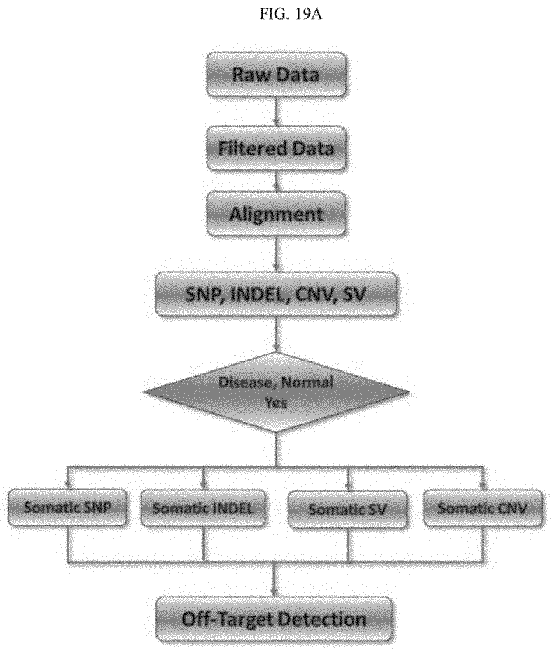

Clustering analysis revealed similar excision patterns with high efficiency across the different tissues in the cohort of animals that received combination treatments in comparison to those detected in the groups that received only CRISPR-Cas9 (FIGS. 16A-16C). This hierarchical clustering heat map may offer a predictive capability for viral elimination after the interruption of LASER ART in this model. Bioinformatics analysis of human genome sequence data identified several human genome sites that may serve as targets for gRNAs that are designed for editing of HIV-1 DNA. However, results from sequencing of several selected sites with high scores of specificities and/or their locations in the exons ruled out any off-target effect on genome of human cell line (FIGS. 17A-17C and 18A-18F). Further, deep sequencing of genomic DNA from spleen of the four treated animals, including two that showed no rebound after combination treatment and one from each group with single treatment followed by bioinformatics analysis in search for somatic InDels mutation in the human genome by multiple alignments involving nucleic acids blast revealed no off target effects such as single nucleotide variations, translocation, inversion, deletion, tandem duplication, and insertion in the human genome, that can be attributed to CRISPR-Cas9 (FIGS. 19A-19C, 20A-20D and Tables 6, 7).

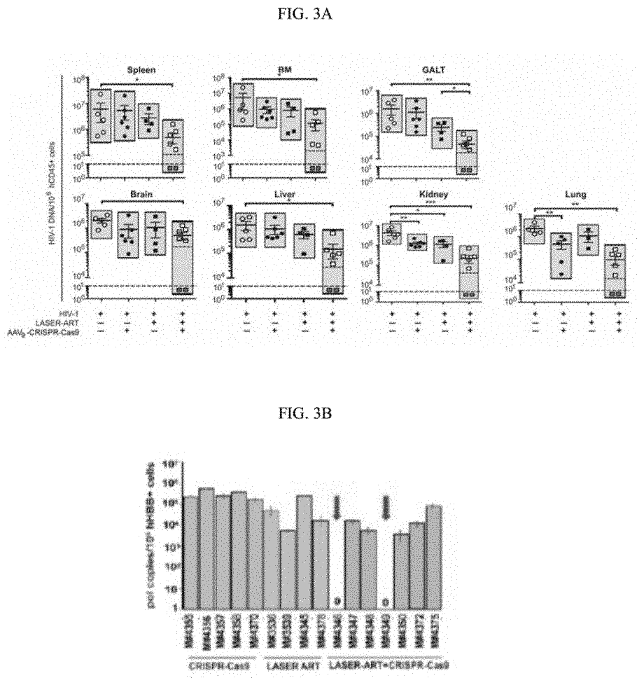

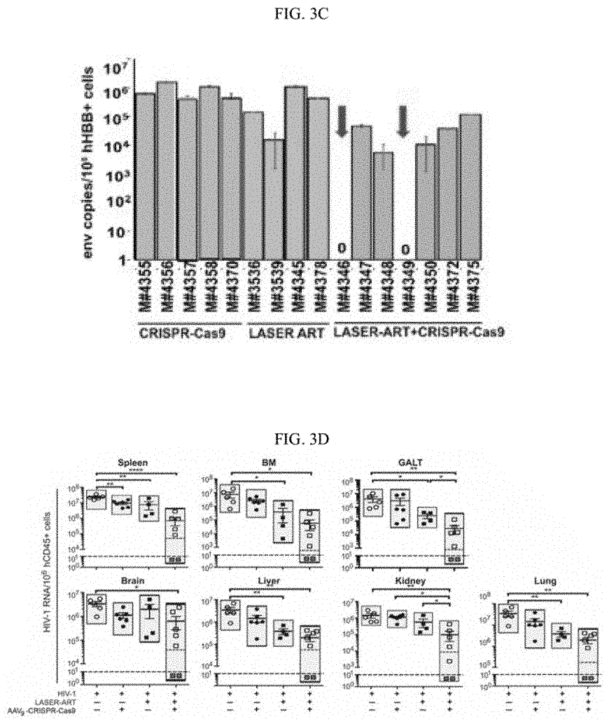

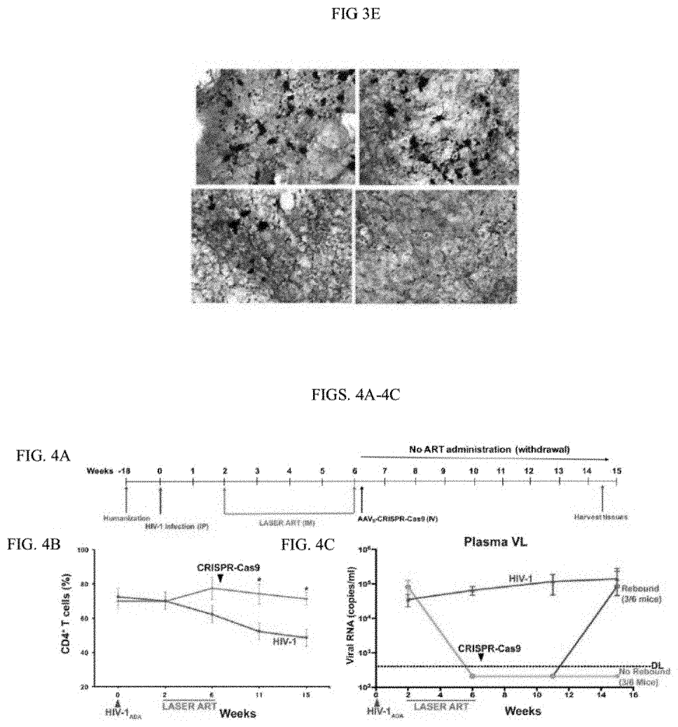

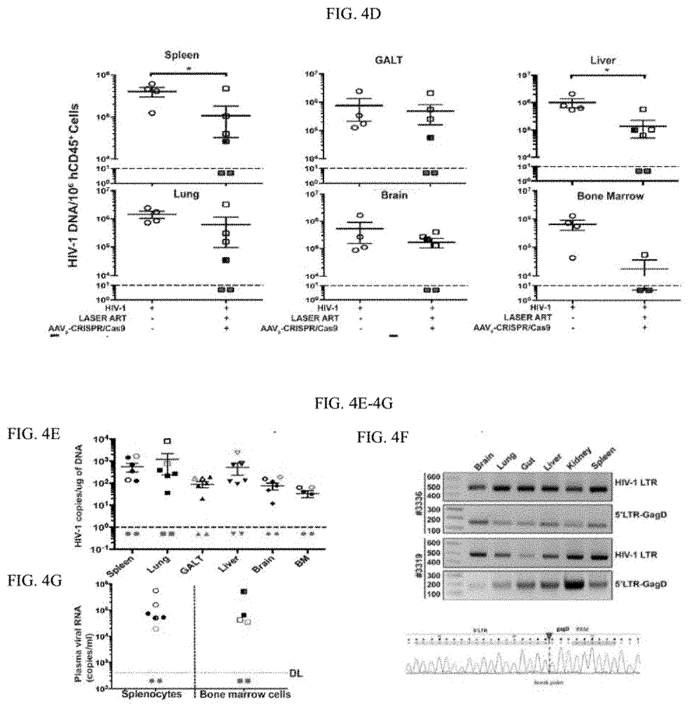

Next, tissue viral DNA and RNA levels were determined in tissues using ultrasensitive semi-nested real time qPCR with primers and probes designed for detection of HIV-1 gag (21, 22) DNA analysis results revealed that combination treatment was more effective than either LASER ART or CRISPR-Cas9 alone in reducing viral DNA copies. The spleen, GALT, and bone marrow of mice #4346 and #4349 showed no rebound (FIG. 3A). Similarly, results from the RNA detection assay corroborated with the data from the DNA study showed the combination of LASER ART and CRISPR-Cas9 reduced HIV-1 RNA production in select animals with complete absence of viral RNA in #4346 and #4349 (FIG. 3B). The presence of HIV-1 RNA was also examined by RNA scope using 10 μm thick spleen sections from infected animals and antisense probe V-HIV-1 Clade-B designed for targeting 854-8291 base pairs of the HIV-1_{NL4-3}. Mouse #4346 with no viral nucleic acid and rebound showed no evidence of viral gene expression (FIG. 4A). Results from the targeted qPCR for DNA sequence detection corresponded to the middle of HIV-1 genome and ruled out the presence of DNA corresponding to the pol and env genes (FIGS. 4B-4C). Additional evidence for the absence of HIV-1 genomes in the animals #4346 and #4349 was provided by digital droplet PCR (ddPCR) tests. This assay had a sensitivity of detection of <2 viral copies. Verifying prior results, no viral DNA was detected in spleens of mice #4346 and #4349 and examination of other tissues showed complete HIV-1 eradication (FIGS. 4D-4E). Finally, a viral rescue assay was performed by co-culturing bone marrow cells and splenocytes of representative samples with PHA/IL-2 PBMCs for an additional two weeks. Representative data (FIG. 4F) showed that while HIV-1 was rescued from 100% of samples with detectable viral DNA and RNA, despite the presence of high number of human cells, no evidence for virus recovery was observed in the samples from the two animals with eradicated HIV-1 DNA and RNA.

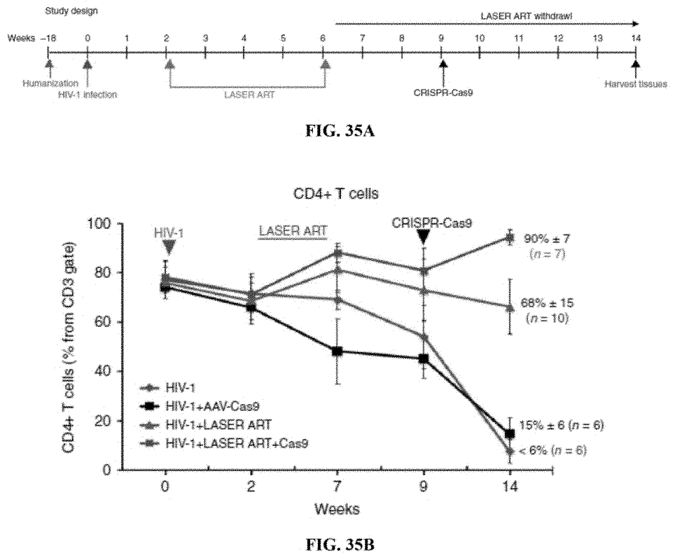

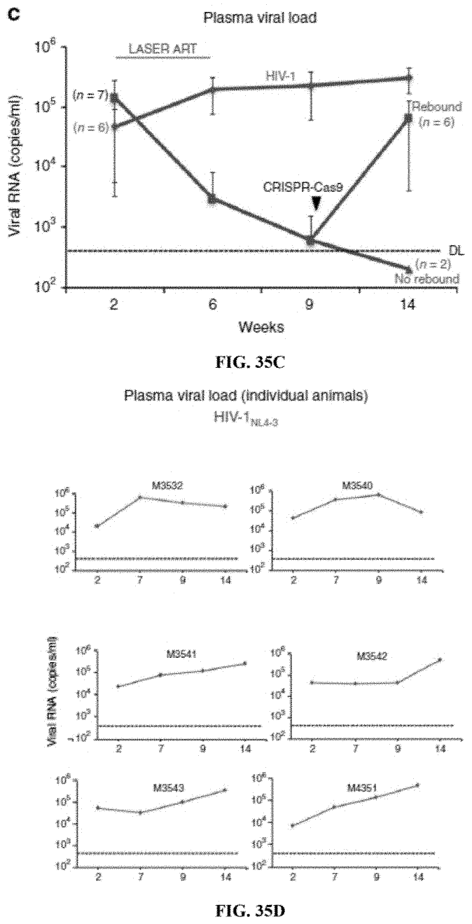

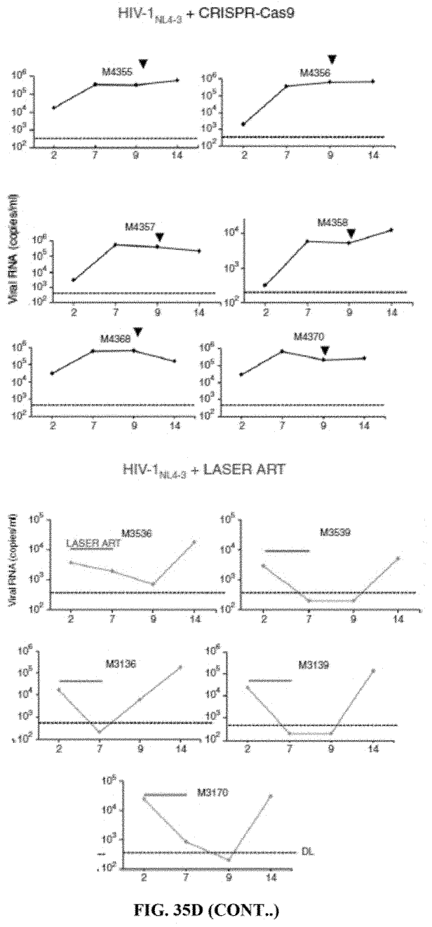

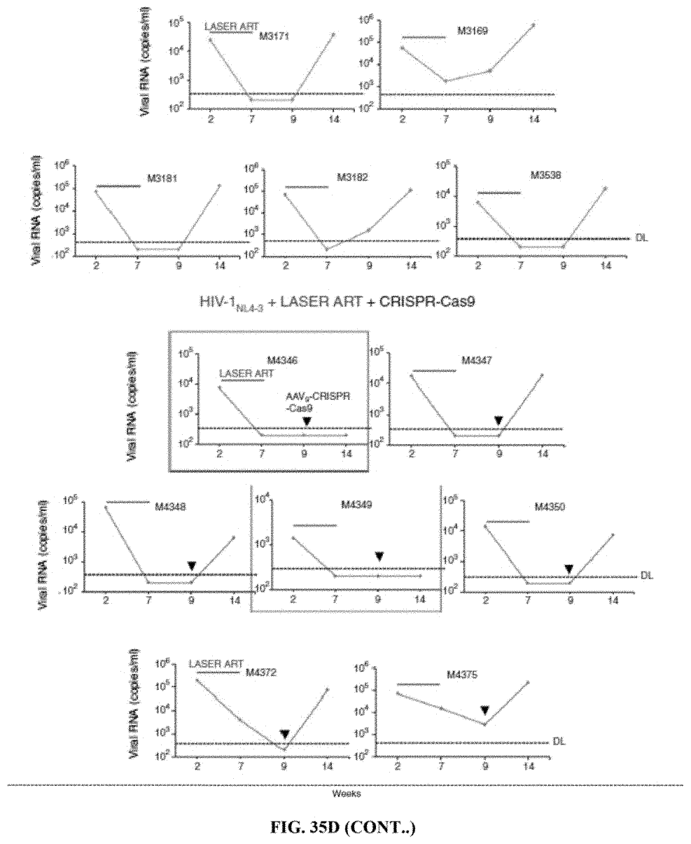

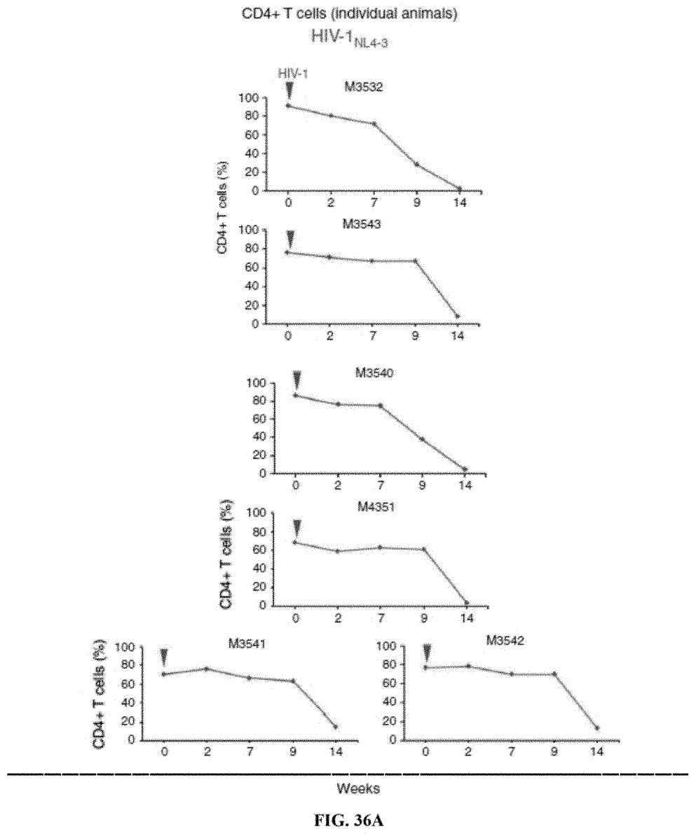

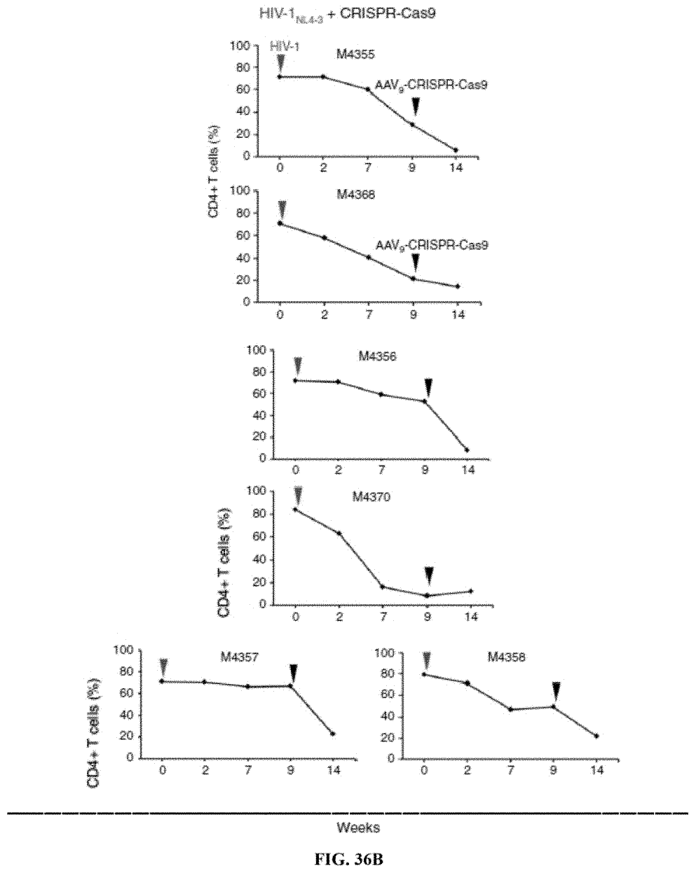

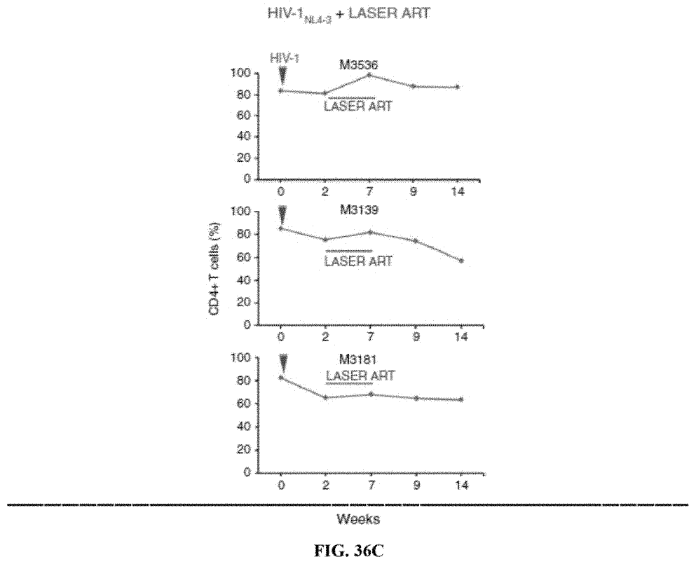

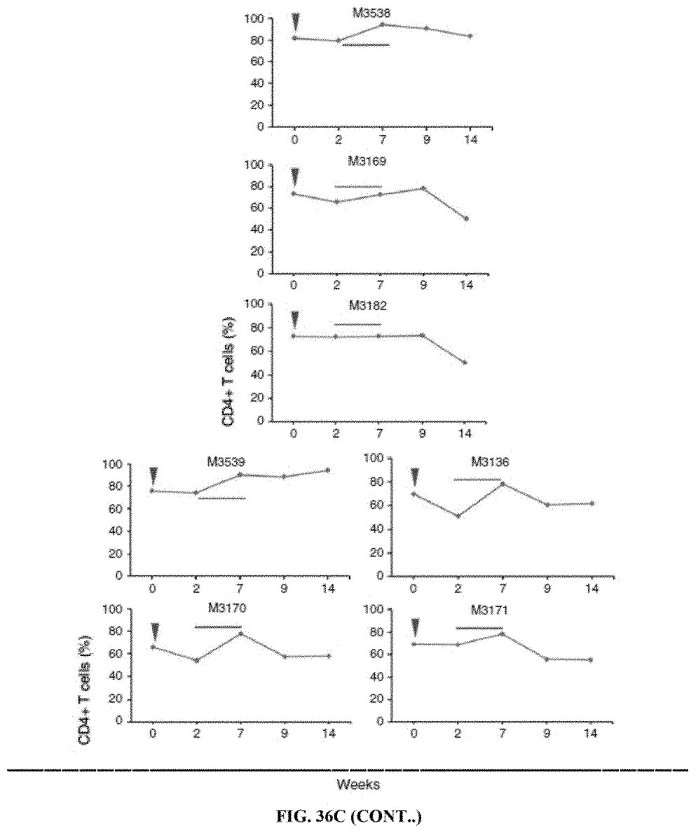

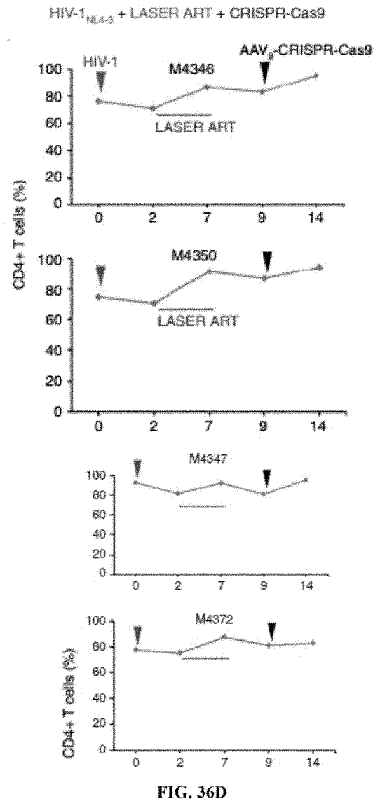

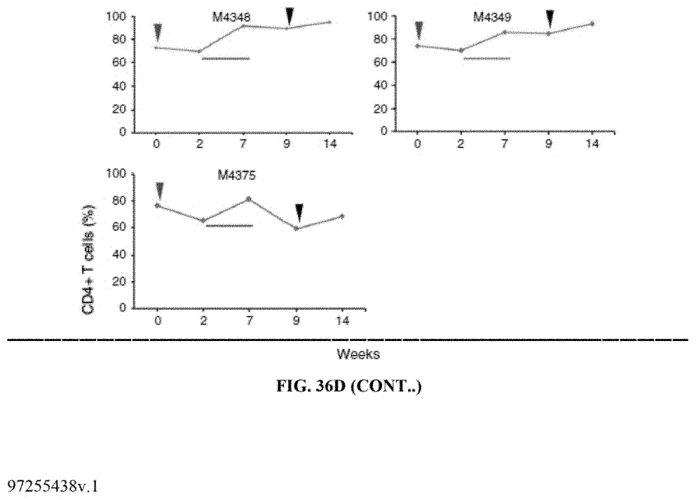

Viral rebound after LASER ART and AAV₉-CRISPR-Cas9 treatment of infected humanized mice. With the model and therapies in hand, the ability of LASER ART and CRISPR-Cas9 to affect viral rebound after therapeutic interruption in HIV-1 infected humanized mice was evaluated (FIG. 35A). In these experiments, HSC reconstituted NSG mice (n=33) were infected with 10⁴ TCID50 of HIV-1_{NL4-3} for 2 weeks. Four representative animals were sacrificed at this time point to confirm viral infection establishment from various tissues. At this time, depletion of CD4⁺ T cells (FIG. 35B) was coincident with plasma viral RNA at a median of 2.2×10⁵ copies/ml (FIG. 35C). The remaining 29 HIV-1 infected animals were divided into four groups with four more uninfected untreated animals serving as uninfected controls. The first group (n=6) of mice were left untreated (HIV-1 control), the second group (n=6) received a single intravenous (IV) injection of AAV₉-CRISPR-Cas9, 10¹² GC (genome copy) units, with a volume of 50 μl; the third group (n=10) were administered LASER ART that consisted of 45 mg/kg parent drug equivalents of nanoformulated RPV and myr-istoylated DTG, and 40 mg/kg parent drug equivalents of myr-istoylated 3TC and ABC nanoparticles by intramuscular (IM) injection. A fourth group (n=7) received LASER ART followed by AAV9-CRISPR-Cas9. Eight weeks following the last administration of LASER ART and five weeks after the single AAV9-CRISPR-Cas9 treatment animals were observed for evidence of viral rebound (FIG. 35C). In the group that received LASER ART with subsequent AAV9-CRISPR-Cas9, viral rebound was not observed in two animals. Examination of the plasma viral load (FIG. 35D) for each individual animal showed drastic decline in the viral copy number to below detectable levels in the group of animals treated with LASER ART. Removal of LASER ART led to rebound in all 10 animals treated with LASER ART alone and in five out of seven animals that received both LASER ART and AAV9-CRISPR-Cas9. Repeated search for the viral RNA in the plasma of two animals, M4346 and M4349 (FIG. 35D framed in red), failed to detect evidence of viral presence. In the absence of LASER ART, numbers of CD4⁺ T cells relative15±6% and <6% in groups 2 and 1, respectively (FIG. 35B). The CD4⁺ T cell profile of each animal is shown (FIGS. 36A-36D) for all treatment groups. Disease was determined by declining percentages of CD4⁺ T cells. Results showed a robust restoration of CD4⁺ T cells in the animals that received LASER ART alone or in combination with AAV9-CRISPR-Cas9 as compared to infected controls and AAV9-CRISPR-Cas9 alone treated animals (FIGS. 35B and 36A-36D).

Next, the number of total human cells (CD45) and T cells (CD3⁺) were evaluated by flow cytometry and demonstrated sustained human cell numbers in both control (uninfected), infected and treated animals at and beyond four months until the study conclusion (FIGS. 37A, 37B respectively). The presence of human CD4⁺ cells (FIG. 35B) and HLA-DR in spleen was observed to confirm graft stability. We also observed restoration of CD4⁺ T cells in spleens of dual-treated animals (FIG. 35B). This was further confirmed by the identification of species-specific DNA sequences in spleens of all animal groups independent of treatments administered (FIG. 35C). Indeed, cell numbers proved constant following all CRISPR-Cas9 and LASER ART interventions.

Accordingly, these results provide evidence that the combination of lipophilic LASER ART and AAV₉ delivered CRISPR-Cas9 can lead to the cure of HIV-1 infection by elimination of the replication component of the virus in HIV-1 reservoirs of infected animals as evidenced by the absence of viremia for more than 8 weeks after the last ART treatment. Although the re-appearance of viremia in humans can occasionally be delayed longer (5), the rebound of HIV occurs an average of 2-4 weeks after ART interruption (4, 23) and 5-9 days in animal models (24). These results offer a realistic pathways toward an HIV-1 cure.

TABLE 1
PCR primers and probes

| | |
|---|---|
| primer | sequence |

1. Standard PCRs

| | | |
|---|---|---|
| 5′LTR-gag | 1^{st} round LTR F | 5′- |
| | | AATTGCGGCCGCTGGAAGGGCTAATT |
| | | TGGTCCC-3′ (SEQ ID NO: 1) |
| | 1^{st} round gag R | 5′-TGTCACTTCCCCTTGGTTCTCTC-3′ |
| | | (SEQ ID NO: 2) |
| | nested 5′LTR F | 5′- |
| | | AAAAGAATTCGTGGATCTACCACACA |
| | | CAAGGC-3′ (SEQ ID NO: 3) |
| | nested gag R | 5′- |
| | | AAAAGGATCCACCATTTGCCCCTGGA |
| | | GGTT-3′ (SEQ ID NO: 4) |
| Gag-3′LTR | 1^{st} round gag F | 5′- |
| | | GAAAGCGAAAGTAAAGCCAGAGGAG |
| | | AT-3′ (SEQ ID NO: 5) |
| | 1^{st} round LTR R | 5′- |
| | | ACACAACAGACGGGCACACACTACTT-3′ |
| | | (SEQ ID NO: 6) |
| | nested gag F | 5′AAAAGAATTCGACAGCTACAACCAT |
| | | CCCTTCAGACAG-3′ (SEQ ID NO: 7) |
| | nested 3'LTR R | 5′- |
| | | AAAAGGATCCAGCAGTGGGTTCCCTA |
| | | GTTAGCCAG-3′ (SEQ ID NO: 8) |
| LTRs | 1^{st} round LTR −413/S | 5′-TTGGCAGAACTACACACCAGGG-3′ |
| | | (SEQ ID NO: 9) |
| | 1^{st} round LTR +43/AS | 5′-CCGAGAGCTCCCAGGCTCAGATCT- |
| | | 3′ (SEQ ID NO: 10) |
| | nested LTR −374/S | 5′-TTAGCAGAACTACACACCAGGGCC- |
| | | 3′ (SEQ ID NO: 11) |
| | nested LTR −19/AS | 5′-GCTGCTTATATGTAGCATCTGAG-3′ |
| | | (SEQ ID NO: 12) |
| Hs beta-globin | Hs b-globin F | 5′-CCCTTGGACCCAGAGGTTCT-3′ |
| | | (SEQ ID NO: 13) |
| | Hs b-globin R | 5′-CGAGCACTTTCTTGCCATGA-3′ |
| | | (SEQ ID NO: 14) |
| Mm beta-globin | Mm b-globin F | 5′-CCCTTGGACCCAGCGGTACT-3′ |
| | | (SEQ ID NO: 15) |
| | Mm b-globin R | 5′-GTTATCACCTTCTTGCCATG-3′ (SEQ |
| | | ID NO: 16) |

2. Taqman qPCRs

| | | |
|---|---|---|
| pol | HIV-1 pol/int F | 5′-TCCAGCAGAGACAGGGCAAG-3′ |
| | | (SEQ ID NO: 17) |
| | HIV-1 pol/int R | 5′-TGCCAAATTCCTGCTTGATCCC-3′ |
| | | (SEQ ID NO: 18) |
| | HIV-1 pol/int probe | 5′-HEX- |
| | | CGCCCACCAACAGGCGGCCTTAACTG- |
| | | ZEN-IowaBlackFQ-3′ (SEQ ID NO: 19) |
| env | HIV-1 Env F | 5′-TCCTTGGGATGTTGATGATCT-3′ |
| | | (SEQ ID NO: 20) |
| | HIV-1 Env R | 5′-TGGCCCAAACATTATGTACC-3′ |
| | | (SEQ ID NO: 21) |
| | HIV-1 Env Probe | 5′-FAM- |
| | | TGGTGGTTGCTTCTTTCCACACA-ZEN- |
| | | IowaBlackFQ-3′ (SEQ ID NO: 22) |
| reference | Hs β-g1obin F | 5′-CCCTTGGACCCAGAGGTTCT-3′ |
| | | (SEQ ID NO: 23) |
| | Hs β-g1obin R | 5′-CGAGCACTTTCTTGCCATGA-3′ |
| | | (SEQ ID NO: 24) |
| | Hs β-g1obin probe: | 5′-FAM- |
| | | GCGAGCATCTGTCCACTCCTGATGCTG |
| | | TTATGGGCGCTCGC-ZEN-IowaBlackFQ- |
| | | 3′ (SEQ ID NO: 25) |

3. RT-PCRs

| | |
|---|---|
| LTR1/F | 5′-GCAGAACTACACACCAGGGCC-3′ |
| | (SEQ ID NO: 26) |
| GagD/F | 5′-GGATAGATGTAAAAGACACCA-3′ |
| | (SEQ ID NO: 27) |
| pX601gRNAscaffold/ | 5′-CGCCAACAAGTTGACGAGAT-3′ |
| R | (SEQ ID NO: 28) |
| SaCas9/263/F | 5′-TCGACTACAACCTGCTGACC-3′ |
| | (SEQ ID NO: 29) |
| SaCas9/SEQ1 | 5′-GGTGGGCTTCTTCTGCTT-3′ (SEQ ID |
| | NO: 30) |
| b- actin S | 5′-CTACAATGAGCTGCGTGTGGC-3′ |
| | (SEQ ID NO: 31) |
| b-actin AS | 5′-CAGGTCCAGACGCAGGATGGC-3′ |
| | (SEQ ID NO: 32) |

4. In vitro OFF target analysis

| | | |
|---|---|---|
| LTR 1 OFF | LTR1OFFch8/F | 5′-GAGTGACCTTCCCAAATTGC-3′ |
| targets | | (SEQ ID NO: 34) |
| | LTR1OFFch8/R | 5′-ATGGTGAGGTGAGGGATGAG-3′ |
| | | (SEQ ID NO: 35) |
| | TSC2/35001F | 5′-CAGACTCTGATGGGTGGCAG-3′ |
| | | (SEQ ID NO: 36) |
| | TSC2/35398R | 5′-GCTAAGGAGAGAGGGTGGGA-3′ |
| | | (SEQ ID NO: 37) |
| | TUB/66607F | 5′-CCAAGTGGCCCTCAGATTACA-3′ |
| | | (SEQ ID NO: 38) |
| | TUB/67015R | 5′-TCATTCACCCCAAATCCTACGG-3′ |
| | | (SEQ ID NO: 39) |
| Gag D OFF | GagDOFFch3/F | 5′-CATTAACCACCTGGGGAACA-3′ |
| targets | | (SEQ ID NO: 40) |
| | GagDOFFch3/R | 5′-TCTCAGACCCAGGAATGTCA-3′ |
| | | (SEQ ID NO: 41) |
| | TACC2/392F | 5′-GAGGACTCTCCAGCCAAAGG-3′ |
| | | (SEQ ID NO: 42) |
| | TACC2/782R | 5′-GAGCTGGGGGTCTTAGAGGA-3′ |
| | | (SEQ ID NO: 43) |
| | ADNP/41574F | 5′-TGCACCAGCCAAAACTTAGGA-3′ |
| | | (SEQ ID NO: 44) |
| | ADNP/41996R | 5′-TCTAATTAGGTGGCAGCACGTT-3′ |
| | | (SEQ ID NO: 45) |

TABLE 2
(Table discloses SEQ ID NOS 48-97 and 97, respectively, in order of appearance)

| HIV-1 LTR 1 target (+strand) | | | | | | | | On- |
|---|---|---|---|---|---|---|---|---|
| GCAGAACTACACACCAGGGCCAGGGAT | | | | | | | Mis- | tar- |
| Sequence | PAM | Score | Gene | Chromosome | Strand | Position | match | get |
| TCTAAACTCCACACCAGGGCC | ATGAA | 2.6 | | chr8: +22915337 | 1 | 22915337 | 4 | False |
| TCAGATCTCCACACCAGAGCC | ACGAG | 1.3 | | chr9: +38360364 | 1 | 38360364 | 4 | False |
| ACAGGCCAACCCACCAGGGCC | CAGAG | 0.9 | | chr22: −20136959 | −1 | 20136959 | 5 | False |
| GTAGGACTACGCACCAGGGCA | AAGAG | 0.9 | | chr8: −92102695 | −1 | 92102695 | 4 | False |
| ACAAAAGTACACACCAGAGCC | TGGGG | 0.8 | | chr11: +75625035 | 1 | 75625035 | 4 | False |
| TGTGAACTACGCCCCAGGGCC | TGGAA | 0.8 | | chr13: −27341725 | −1 | 27341725 | 5 | False |
| ACAGAGCTGAGCACCAGGGCC | CAGGG | 0.8 | | chr10: +124217737 | 1 | 1.24E+08 | 5 | False |
| CCAGTTCTCCACCCCAGGGCC | ATGGA | 0.8 | | chr15: +28948038 | 1 | 28948038 | 5 | False |
| CCAGAGCTGCTTACCAGGGCC | ATGGA | 0.7 | | chr1: −47650696 | −1 | 47650696 | 5 | False |
| ACAGCACTCCCCACCAGGGCT | TGGGG | 0.7 | TSC2 | chr16: −2082981 | −1 | 2082981 | 5 | False |
| | | | (ENSG00000103197) | | | | | |
| ACAGAACGTCACACCAGGGTC | AGGAG | 0.7 | | chr7: +26573832 | 1 | 26573832 | 4 | False |
| ACAAAACTAGACAGCAGGGCC | AGGAG | 0.7 | | chr19: −54347353 | −1 | 54347353 | 4 | False |
| TGAGCACTTCACAGCAGGGCC | GGGAA | 0.7 | | chr2: +43112424 | 1 | 43112424 | 5 | False |
| GCAGCACTACACATCAGGGCT | AAGAA | 0.7 | | chr16: −60058984 | −1 | 60058984 | 3 | False |
| CCGCAACTCCACAGCAGGGCC | AGGGA | 0.7 | | chr15: +80851232 | 1 | 80851232 | 5 | False |
| CTAGAGGAACACACCAGGGCC | TGGGA | 0.6 | | chrX: −103784275 | −1 | 1.04E+08 | 5 | False |
| ACAGCCCCAGACACCAGGGCC | TGGAG | 0.6 | | chr15: −57542258 | −1 | 57542258 | 5 | False |
| CCAGGTCTACCCAGCAGGGCC | AGGAG | 0.6 | | chr11: +121718784 | 1 | 1.22E+08 | 5 | False |
| ACAGGAGGGCACACCAGGGCC | CAGGA | 0.6 | | chr13: −47252260 | −1 | 47252260 | 5 | False |
| ACAGAAATAAACACCAGGGCT | TCGGG | 0.6 | | chr2: −12064330 | −1 | 12064330 | 4 | False |
| GCAGAACTGCAGACCAGGGGC | TGGGG | 0.6 | | chr11: −76235199 | −1 | 76235199 | 3 | False |
| CCAGAGCACCAAACCAGGGCC | CAGGA | 0.5 | | chr2: −238434996 | −1 | 2.38E+08 | 5 | False |
| GCAGAGCTCCCCACCAGGGGC | AGGGA | 0.5 | | chr2: −127586882 | −1 | 1.28E+08 | 4 | False |
| ACAGGCCCACACTCCAGGGCC | CAGAA | 0.5 | | chr5: −134248836 | −1 | 1.34E+08 | 5 | False |
| GCAGTGCCACACTCCAGGGCC | TTGGG | 0.5 | | chr19: −11048922 | −1 | 11048922 | 4 | False |
| GCAGGAGTAGGCACCAGGGCC | CTGAG | 0.5 | | chr1: −41442886 | −1 | 41442886 | 4 | False |
| GCAGCACCACACACCAGGCCC | AGGAG | 0.5 | | chr14: −96723779 | −1 | 96723779 | 3 | False |
| GCAGAGCTAGCCACCAGGGCT | TAGGA | 0.4 | | chr6: −137609940 | −1 | 1.38E+08 | 4 | False |
| GCAGAGCTCCAGCCCAGGGCC | TGGGG | 0.4 | | chr22: +49956739 | 1 | 49956739 | 4 | False |
| GGGGAAATACACATCAGGGCC | AGGAA | 0.4 | | chr20: −43964342 | −1 | 43964342 | 4 | False |
| AGAGAATTTCACAACAGGGCC | CTGAA | 0.4 | | chr3: −189039375 | −1 | 1.89E+08 | 5 | False |
| ACAAACCTACAGACCAGAGCC | CAGGG | 0.4 | TUB | chr11: −8105357 | −1 | 8105357 | 5 | False |
| | | | (ENSG00000166402) | | | | | |
| CATGAGCTACACACCAGGACC | AGGAG | 0.4 | | chr7: +47512275 | 1 | 47512275 | 5 | False |
| GAAAAACTACAGACCAGGGAC | AAGGG | 0.4 | | chr6: −68746690 | −1 | 68746690 | 4 | False |
| CCAGAACTCAGCCCCAGGGCC | CTGGG | 0.4 | | chr5: −137113375 | −1 | 1.37E+08 | 5 | False |
| GCTGGCCTACACACCAGGCCC | AGGGG | 0.4 | | chr3: +38015758 | 1 | 38015758 | 4 | False |
| CCTGAACCACACCCCAGGGCT | CAGGG | 0.3 | | chr2: −128441625 | −1 | 1.28E+08 | 5 | False |
| GCAGAACACCAAGCCAGGGCC | AGGAA | 0.3 | | chr10: −95607490 | −1 | 95607490 | 4 | False |
| GAATAGCTACACACTAGGGCC | ATGGA | 0.3 | | chr2: −69175215 | −1 | 69175215 | 4 | False |
| GAAGAACCACAAAACAGGGCC | CAGAA | 0.3 | | chrX: +43803871 | 1 | 43803871 | 4 | False |
| ATAGTACTACACTCCTGGGCC | TCGAG | 0.3 | | chr5: +5184057 | 1 | 5184057 | 5 | False |
| GAAGAACAACACAGCAGGGCA | GAGAG | 0.2 | TBC1D19 | chr4: +26576719 | 1 | 26576719 | 4 | False |
| | | | (ENSG00000109680) | | | | | |
| CCAGAAACACCCACCAGTGCC | CGGGA | 0.2 | | chr19: +15265258 | 1 | 15265258 | 5 | False |
| CCAGAGCTGCAGACCCGGGCC | CCGGG | 0.2 | | chr9: −133679870 | −1 | 1.34E+08 | 5 | False |
| CCAGACCGAGACACCAGGGGC | GGGGG | 0.2 | SLC41A2 | chr12: +104958165 | 1 | 1.05E+08 | 5 | False |
| | | | (ENSG00000136052) | | | | | |
| CCAGATCTAGACTCCAGGGCA | GTGAG | 0.2 | | chr1: −203423414 | −1 | 2.03E+08 | 5 | False |
| TCAGAGCTAGACTCCAGGGCT | GGGGG | 0.2 | | chr19: −48393411 | −1 | 48393411 | 5 | False |
| GGAGAACTTAACACCAGGTCC | CTGGG | 0.2 | | chr22:: −41003477 | −1 | 41003477 | 4 | False |
| CCAGCACCACAGAGCAGGGCC | TGGGA | 0.2 | | chr11: +319276 | 1 | 319276 | 5 | False |
| CCAGCACCACAGAGCAGGGCC | TGGGA | 0.2 | | chr11: −310505 | −1 | 310505 | 5 | False |

TABLE 3
(Table discloses SEQ ID NOS 98-137, 137, 137, 137 and 137-144,
respectively, in order of appearance)

| HIV-1 Gag D target (+strand) | | | | | | | | On- |
|---|---|---|---|---|---|---|---|---|
| GGATAGATGTAAAAGACACCAAGGAAG | | | | | | | Mis- | tar- |
| Sequence | PAM | Score | Gene | Chromosome | Strand | Position | match | get |
| AGAAAAATGTAAAAGACACCT | TGGAA | 1.7 | | chr3: −144746442 | −1 | 1.45E+08 | 4 | FALSE |
| TTATACATTTGAAAGACACCA | AAGAA | 1.5 | | chr1: −194738918 | −1 | 1.95E+38 | 5 | FALSE |
| GGATAAATGGGAAAGACACCA | GGGGA | 1.5 | | chr16: −48814775 | −1 | 48814775 | 3 | FALSE |
| TCTTAGACTTAAAAGACACCA | TTGAA | 1 | | chr15: −33069866 | −1 | 33069866 | 5 | FALSE |
| ACATTGAATTAAAAGACACCA | TAGAG | 1 | | chrX: −32002168 | −1 | 32002168 | 5 | FALSE |
| GGATAGAGCCAAAAGACACCA | AAGAG | 1 | | chr17: −51350241 | −1 | 51350241 | 3 | FALSE |
| AAATAGCTCTTAAAGACACCA | GCGAA | 0.9 | | chr2: +173764948 | 1 | 1.74E+08 | 5 | FALSE |
| AGATCAATGTAAAAGTCACCA | TCGAA | 0.9 | | chr6: −144452168 | −1 | 1.44E+08 | 4 | FALSE |
| TTTTAGATGTAAAAGACATCA | GGGAG | 0.8 | | chr3: +187644948 | 1 | 1.88E+08 | 4 | FALSE |
| TGATAAATGAAACAGACACCA | GAGGA | 0.8 | | chr7: −141719859 | −1 | 1.42E+08 | 4 | FALSE |
| GAAAAGATTTAAGAGACACCA | AAGAG | 0.8 | | chr2: −213166139 | −1 | 2.13E+08 | 4 | FALSE |
| AGGGAGATCTAAGAGACACCA | GAGAG | 0.8 | | chr19: −29842353 | −1 | 29842353 | 5 | FALSE |
| ATGCAGATGTAACAGACACCA | GGGAA | 0.8 | | chr1: −226725698 | −1 | 2.27E+08 | 5 | FALSE |
| GTATGGATGTTAAAGACTCCA | TTGAG | 0.7 | | chr5: −142976527 | −1 | 1.43E+08 | 4 | FALSE |
| CGGTAGATTTTAAAGACTCCA | AAGAG | 0.7 | | chr9: −38648188 | −1 | 38648188 | 5 | FALSE |
| AGAGAGATATTAAAGACCCCA | GTGAA | 0.6 | | chr18: −43665283 | −1 | 43665283 | 5 | FALSE |
| GGATAAATGTGAAAGACATCA | TAGAA | 0.6 | | chr18: +51783716 | 1 | 51783716 | 3 | FALSE |
| AGAAGGAGGAAAAAGACACCA | GGGAG | 0.6 | | chr2: +218083925 | 1 | 2.18E+08 | 5 | FALSE |
| TAATAGGTAGAAAAGACACCA | GTGAA | 0.6 | | chr12: −126150002 | −1 | 1.26E+08 | 5 | FALSE |
| CCAAAGATGAAAAAGACACCC | GAGAA | 0.6 | TACC2 | chr10: +122211347 | 1 | 1.22E+−08 | 5 | FALSE |
| | | | (ENSG00000138162) | | | | | |
| TTATAAATGCAAAAGACACCC | ATGAA | 0.6 | | chr14: −46407726 | −1 | 464077265 | | FALSE |
| GGCTGGGTGAAAAAGACACCA | TGGAA | 0.6 | | chr6: +66585737 | 1 | 66585737 | 4 | FALSE |
| GGACAGATGTGAAAGAGACCA | AAGGA | 0.5 | | chr2: +224685483 | 1 | 2.25E+08 | 3 | FALSE |
| TGATGCAAGTAACAGACACCA | TGGGA | 0.5 | | chr6: +107524588 | 1 | 1.08E+08 | 5 | FALSE |
| CAATAGTTGTTCAAGACACCA | GTGAA | 0.5 | | chr6: −156459061 | −1 | 1.56E+08 | 5 | FALSE |
| AGAAAGATACAGAAGACACCA | GGGAG | 0.5 | | chr11: −75223569 | −1 | 75223569 | 5 | FALSE |
| TGAGACTTGTACAAGACACCA | CGGGG | 0.5 | ANDP | chr20: −50889247 | −1 | 50889247 | 5 | FALSE |
| | | | (ENSG00000101126) | | | | | |
| AGATTGTTGGTAAAGACACCA | CAGAG | 0.5 | | chr7: −114527499 | −1 | 1.15E+08 | 5 | FALSE |
| GGAAAGTTATAAAAGACACCG | GGGAA | 0.5 | | chr7: +99103371 | 1 | 99103371 | 4 | FALSE |
| CCATTGATCTAAAAGTCACCA | CTGGA | 0.5 | | chr3: −65736276 | −1 | 65736276 | 5 | FALSE |
| AAATACCTGTAAGAGACACCA | CTGAG | 0.5 | | chr3: −65976946 | −1 | 65976946 | 5 | FALSE |
| TGGTAGATTTATAAGACACCG | TAGGG | 0.5 | | chr10: −3117938 | −1 | 3117938 | 5 | FALSE |
| GAATGGATGTGAAAGGCACCA | CTGAA | 0.5 | | chr5: −79875682 | −1 | 79875682 | 4 | FALSE |
| AAATAAATGTGAAAGTCACCA | CAGAA | 0.5 | | chr8: −131973280 | −1 | 1.32E+08 | 5 | FALSE |
| AGATGGATGGCATAGACACCA | CGGGG | 0.4 | | chr3: +52369345 | 1 | 52369345 | 5 | FALSE |
| TGAAAGATCTTAAAGCCACCA | AAGGA | 0.4 | | chr20: −24138791 | −1 | 24138791 | 5 | FALSE |
| GAGTAGATCTAAAAGACAGCA | AGGAA | 0.4 | | chr12: −62718609 | −1 | 62718609 | 4 | FALSE |
| TCATATGTGTAAAAGACACAA | AGGAG | 0.4 | | chr2: +3551648 | 1 | 3551648 | 5 | FALSE |
| GGTTAGCGGGAAAAGACACCA | CAGGG | 0.4 | | chrX: −141696540 | −1 | 1.42E+08 | 4 | FALSE |
| GGTTAGCGGGAAAAGACACCA | CAGGG | 0.4 | | chrX: +141591639 | 1 | 1.42E+08 | 4 | FALSE |
| GGTTAGCGGGAAAAGACACCA | CAGGG | 0.4 | | chrX: −141582808 | −1 | 1.42E+08 | 4 | FALSE |
| GGTTAGCGGGAAAAGACACCA | CAGGG | 0.4 | | chrX: −141240587 | −1 | 1.41E+08 | 4 | FALSE |
| GGTTAGCGGGAAAAGACACCA | CAGGG | 0.4 | | chrX: +141004580 | 1 | 1.41E+08 | 4 | FALSE |
| GGATTCATGCAAAAGACACTA | TAGGG | 0.4 | | chr3: −85730118 | −1 | 85730118 | 4 | FALSE |
| AGAAATATCTAAAAGACAACA | AAGAG | 0.4 | | chr7: +122888645 | 1 | 1.23E+08 | 5 | FALSE |
| GGAAAGGAGCAAAAGACACCA | GAGGG | 0.4 | | chr17: −81470363 | −1 | 81470363 | 4 | FALSE |
| AGATTCATTTAAAAGACAACA | AAGAA | 0.4 | | chr8: −109514887 | −1 | 1.1E+08 | 5 | FALSE |
| AGAGATATGTATAAGACACAA | TAGGA | 0.3 | | chr2: +212064031 | 1 | 2.12E+08 | 5 | FALSE |
| AGATAGAAATGAAAGACACTA | GTGAA | 0.3 | | chr2: −141095548 | −1 | 1.41E+08 | 5 | FALSE |
| TGATAAATGGGAATGACACCA | GAGAG | 0.3 | | chr4: +146453372 | 1 | 1.46E+08 | 5 | FALSE |

TABLE 4
HIV-1 LTR1 target single cell clone off target analysis. (Table
discloses SEQ ID NOS 48 and 145-147, respectively, in order of appearance)

| | | | | | | | | Number | |
|---|---|---|---|---|---|---|---|---|---|
| | Target sequence: | | | | | | | of | |
| | LTR1 PAM | | | | | | | Se- | In- |
| | GCAGAACTACACACCAGGGCCAGGGAT | Chromosome | | | | | Single | quences | dels |
| OFF | Predicted off | location/ | | | | Mis- | cell | ana- | de- |
| TARGET | target sequence: | gene | Strand | Position | Score | matches | clone | lyzed | tected |
| 1 | ACAGCACTCCCCACCAGGGCTTGGGGG | Ch 16/TSC2 | − | 2082981 | 0.7 | 5 | TOTAL | 26 | 0 |
| | | | | | | | CTRL1 | 5 | 0 |
| | | | | | | | CTRL2 | 3 | 0 |
| | | | | | | | ERAD1 | 3 | 0 |
| | | | | | | | ERAD2 | 3 | 0 |
| | | | | | | | ERAD3 | 3 | 0 |
| | | | | | | | ERAD4 | 3 | 0 |
| | | | | | | | ERAD5 | 3 | 0 |
| | | | | | | | ERAD6 | 3 | 0 |
| 2 | ACAAACCTACAGACCAGAGCCCAGGGT | Ch 11/TUB | − | 8105357 | 0.4 | 5 | TOTAL | 27 | 0 |
| | | | | | | | CTRL1 | 3 | 0 |
| | | | | | | | CTRL2 | 3 | 0 |
| | | | | | | | ERAD1 | 3 | 0 |
| | | | | | | | ERAD2 | 3 | 0 |
| | | | | | | | ERAD3 | 3 | 0 |
| | | | | | | | ERAD4 | 6 | 0 |
| | | | | | | | ERAD5 | 3 | 0 |
| | | | | | | | ERAD6 | 3 | 0 |
| 3 | CCAGACCGAGACACCAGGGGCGGGGA | Ch 12/ | + | 104958165 | 0.2 | 5 | TOTAL | 6 | 0 |
| | | SLC41A2 | | | | | CTRL1 | | |
| | | | | | | | CTRL2 | | |
| | | | | | | | ERAD1 | | |
| | | | | | | | ERAD2 | 1 | 0 |
| | | | | | | | ERAD3 | 2 | 0 |
| | | | | | | | ERAD4 | | |
| | | | | | | | ERAD5 | 2 | 0 |
| | | | | | | | ERAD6 | 1 | 0 |

TABLE 5
HIV-1 GagD target single cell clone off target analysis (Table
discloses SEQ ID NOS 98 and 148-150, respectively, in order of appearance)

| | | | | | | | | Number | |
|---|---|---|---|---|---|---|---|---|---|
| | Target sequence: | | | | | | | of | |
| | gagD PAM | | | | | | | Se- | In- |
| | GGATAGATGTAAAAGACACCAAGGAAG | Chromosome | | | | | Single | quences | dels |
| OFF | Predicted off | location/ | | | | Mis- | cell | ana- | de- |
| TARGET | target sequence: | gene | Strand | Position | Score | matches | clone | lyzed | tected |
| 1 | CCAAAGATGAAAAAGACACCCGAGAAA | Ch 10/TAAC2 | + | 122211347 | 0.7 | 5 | TOTAL | 34 | 0 |
| | | | | | | | CTRL1 | 3 | 0 |
| | | | | | | | CTRL2 | 3 | 0 |
| | | | | | | | ERAD1 | 6 | 0 |
| | | | | | | | ERAD2 | 6 | 0 |
| | | | | | | | ERAD3 | 3 | 0 |
| | | | | | | | ERAD4 | 4 | 0 |
| | | | | | | | ERAD5 | 4 | 0 |
| | | | | | | | ERAD6 | 5 | 0 |
| 2 | TGAGACTTGTACAAGACACCACGGGGC | Ch 20/ADNP | − | 50889247 | 0.4 | 5 | TOTAL | 23 | 0 |
| | | | | | | | CTRL1 | 2 | 0 |
| | | | | | | | CTRL2 | 3 | 0 |
| | | | | | | | ERAD1 | 3 | 0 |
| | | | | | | | ERAD2 | 3 | 0 |
| | | | | | | | ERAD3 | 3 | 0 |
| | | | | | | | ERAD4 | 3 | 0 |
| | | | | | | | ERAD5 | 3 | 0 |
| | | | | | | | ERAD6 | 3 | 0 |
| 3 | AGAAAAATGTAAAAGACACCTTGGAAA | Ch 3/ | − | 144746442 | 0.2 | 5 | TOTAL | 24 | 0 |
| | | non gene | | | | | CTRL1 | 3 | 0 |
| | | | | | | | CTRL2 | 3 | 0 |
| | | | | | | | ERAD1 | 3 | 0 |
| | | | | | | | ERAD2 | 3 | 0 |
| | | | | | | | ERAD3 | 3 | 0 |
| | | | | | | | ERAD4 | 3 | 0 |
| | | | | | | | ERAD5 | 3 | 0 |
| | | | | | | | ERAD6 | 3 | 0 |

TABLE 6
Number of somatic SNPs in different genomic regions

| Sample | #4349 | #4346 | #4356 |
|---|---|---|---|
| CDS | 6590 | 6760 | 5895 |
| Synonymous_SNP | 3003 | 3053 | 2588 |
| Missense_SNP | 3372 | 3465 | 3050 |
| Stopgain | 70 | 86 | 128 |
| Stoploss | 6 | 6 | 7 |
| Unknown | 140 | 151 | 122 |
| Intronic | 414960 | 415812 | 382465 |
| UTR3 | 8195 | 8197 | 7713 |
| UTR5 | 1905 | 1947 | 1649 |
| Splicing | 23 | 27 | 30 |
| ncRNA_exonic | 3982 | 4073 | 3509 |
| ncRNA_intronic | 65272 | 65664 | 59525 |
| ncRNA_splicing | 14 | 14 | 13 |
| Upstream | 6962 | 7052 | 6063 |
| Downstream | 7822 | 7850 | 7005 |
| Intergenic | 664364 | 669294 | 602974 |
| Total | 1180363 | 1186971 | 1077074 |

Sample: sample name
CDS: the number of somatic SNPs in coding region
Synonymous_SNP: a single nucleotide change that does not cause an amino acid change
Missense_SNP: a single nucleotide change that causes an amino acid change Stopgain: a nonsynonymous SNP that leads to the immediate creation of stop codon at the variant site
Stoploss: a nonsynonymous SNP that leads to the immediate elimination of stop codon at the variant site
Unknown: unknown function (due to various errors in the gene structure definition in the database file)
Intronic: the number of somatic SNPs in intronic region
UTR3: the number of somatic SNPs in 3′UTR region
UTR5: the number of somatic SNPs in 5′UTR region
Splicing: the number of somatic SNPs within 2-bp of a splicing junction
ncRNA_exonic: the number of somatic SNPs in exonic region of non-coding RNAs
ncRNA_intronic: the number of somatic SNPs in intronic region of non-coding RNAs
ncRNA_splicing: the number of somatic SNPs within 2-bp of a splicing junction of non-coding RNAs
Upstream: the number of somatic SNPs within 1 kb away from the transcription start site
Downstream: the number of somatic SNPs within the 1 kb away from the transcription termination site
Intergenic: the number of somatic SNPs in intergenic region
Total: the total number of somatic SNPs

TABLE 7
Number of somatic InDels in different genomic regions

| Sample | #4349 | #4346 | #4356 |
|---|---|---|---|
| CDS | 103 | 124 | 59 |
| Frameshift_deletion | 31 | 34 | 21 |
| Frameshift_insertion | 14 | 16 | 10 |
| Nonframeshift_deletion | 36 | 48 | 16 |
| Nonframeshift_insertion | 18 | 22 | 9 |
| Stopgain | 2 | 2 | 1 |
| Stoploss | 0 | 0 | 0 |
| Unknown | 2 | 2 | 2 |
| Intronic | 36969 | 39727 | 25080 |
| UTR3 | 946 | 1003 | 640 |
| UTR5 | 134 | 149 | 89 |
| Splicing | 5 | 5 | 3 |
| ncRNA_exonic | 285 | 314 | 190 |
| ncRNA_intronic | 5794 | 6247 | 3971 |
| ncRNA_splicing | 2 | 3 | 1 |
| Upstream | 694 | 771 | 417 |
| Downstream | 879 | 958 | 602 |
| Intergenic | 56749 | 61117 | 38406 |
| Total | 102588 | 110452 | 69477 |

Sample: sample name
CDS: the number of somatic InDels in coding region
Frameshift_deletion: a deletion of one or more nucleotides that cause frameshift changes in protein coding sequence
Frameshift_insertion: an insertion of one or more nucleotides that cause frameshift changes in protein coding sequence
Nonframeshift_deletion: a deletion that does not cause frameshift changes Nonframeshift_insertion: an insertion that does not cause frameshift changes
Stopgain: an insertion or a deletion that leads to the immediate creation of stop codon at the variant site
Stoploss: an insertion or a deletion that leads to the immediate elimination of stop codon at the variant site
Unknown: unknown function (due to various errors in the gene structure definition in the database file)
Intronic: the number of somatic InDels in intronic region
UTR3: the number of somatic InDels in 3′UTR region
UTR5: the number of somatic InDels in 5′UTR region
Splicing: the number of somatic InDels within 2-bp of a splicing junction
ncRNA_exonic: the number of somatic InDels in exonic region of non-coding RNAs
ncRNA_intronic: the number of somatic InDels in intronic region of non-coding RNAs
ncRNA_splicing: the number of somatic InDels within 2-bp of a splicing junction of non-coding RNAs
Upstream: the number of somatic InDels within 1 kb away from transcription start site
Downstream: the number of somatic InDels iwithin 1 kb away from transcription termination site
Intergenic: the number of somatic InDels in intergenic region
Total: the total number of somatic InDels

TABLE 8
Cell and animal PK data sets for the LASER ART nanoformulations

| | | NMDTG | NM3TC | NMABC | NRPV |
|---|---|---|---|---|---|
| Macrophage Uptake, | Maximal prodrug uptake | 74.3 | 10.4 | 11.3 | 31.6 |
| Retention and | (μg/10⁶ cells) | | | | |
| Antiretroviral Activity | Prodrug retention | 10.0 | ND | 5.0 | 17.9 |
| | (μg/10⁶ cells) | | | | |
| | Drug Concentration tested (μM) | 100 | 100 | 100 | 100 |
| | Multiplicity of infections (MOI) | 0.01 | 0.01 | 0.01 | 0.01 |
| | Percent of HIV-1 inhibition (%) | ND | 99 | 99 | 99 |
| Pharmacokinetics | λz (1/day) | 0.0506 | 0.6584 | ND | 0.1274 |
| | t_{1/2} (day) | 13.77 | 1.05 | ND | 5.44 |
| | AUCₗₐₛₜ (day^{a} ng/ml) | 38995.2 | 1187.0 | 315.4 | 13694.9 |
| | AUC_{O-∞} (day^{a} ng/ml) | 40727.9 | 1187.4 | 1513.8 | 13706.7 |
| | AUC % Extrapolation | 4.34 | 0.03 | 79.17 | 0.086 |
| | Vb/F (L/kg) | 22.1 | 64.0 | ND | 25.8 |
| | CL/F (L/day/kg) | 1.1 | 42.1 | ND | 3.3 |
| | MRT_{O-∞} | 14.53 | 2.27 | 5.53 | 3.77 |

Tabular representation of in vitro activity of each of the four nanoformulated long-acting antiretroviral drugs (NMDTG, NM3TC, NMABC, and NRPV). The pharmacokinetic (PK) profile of each of the nanoformulated drugs are illustrated with accompanying doses for mouse testing. The various parameters of PK measurement include terminal rate constant (slowest rate constant), (λz), terminal half- life (t_{1/2}), area under the concentration-time curve (AUC), apparent volume of distribution after IM administration (V_{b}/F), apparent total plasma or serum clearance of drug after injection (CL/F), mean resident time (of the unchanged drug in the systemic circulation) (MRT). Source data are provided as a source data file.
HIV-1_{ADA} challenge 10 days after loading.
ND could not be determined; no significant decline in drug levels from day 1 to day 14 after treatment.
^{a}Doses: Single IM injection into mice; NMDTG, NMABC and NRPV = 45 mg/kg as DTG, ABC and RPV equivalents; NM3TC = 50 mg/kg as 3TC equivalents.

Discussion

While ART has transformed HIV-1 infection into a chronic treatable disease, virus persists in tissues that include the gut, lymph nodes, brain, spleen amongst other sites. The inability of ART to eliminate virus in these tissue sanctuaries remains the major obstacle towards a disease cure. Such a limitation is linked, in large measure, to continuous long-term infections in CD4⁺ memory T cells and less frequently in mononuclear phagocytes despite both directed host antiviral immunity and ART effectiveness. Thus, one may predict that, any or all steps towards HIV elimination must include precise targeted ART delivery, maintenance of vigorous immune control, effective blockade of viral growth and immune-based elimination of pools of infected cells or genome integrated proviral DNA. Even under these conditions, the presence of replication competent virus that allows low-levels of viral production and viral latency underscores employment of strategies that eliminate virus that is integrated but latent. Because of notable graft versus host disease in several humanized animal models, examinations for time periods measured in months are limited. In order to overcome the challenge of sustained human grafts in mice, NSG-humanized mice transplanted at birth with HSC were used. Both human myeloid and lymphoid lineages were successfully reconstituted in these mice and support the evaluations of HIV-1 persistence, treatment, and immune functions. The sustained human grafts as confirmed by flow cytometry were viable and functional for more than 6 months, which provided a platform that allowed treatment interventions for prolonged time periods and a clear ability during ART to best establish a continuous latent HIV-1 reservoir in peripheral tissues and the brain and the noted immunological responses to the viral infection. These previously published data support the successful use of humanized mice in studies of HIV/AIDS pathogenesis, therapeutics (Gautam N, et al. Antimicrob. Agents Chemother. 2013; 57:3110-3120. Batrakova E. V., Gendelman H. E., Kabanov A. V. Expert Opin. Drug Deliv. 2011; 8:415-433. McMillan J, Batrakova E, Gendelman H E. Cell delivery of therapeutic nanoparticles. Prog. Mol. Biol. Transl. Sci. 2011; 104:563-601), and treatment (Kadiu I, Nowacek A, McMillan J, Gendelman H E. Nanomedicine. 2011; 6:975-994. Guo D, et al. J. Virol. 2014;88:9504-9513).

The successful outcome of the studies herein, reflects the combinatorial use of a suitable animal model, control of viral set points, reach to the viral reservoirs, delivery and intracellular drug penetration of potent LASER ART, and the widespread employment of CRISPR-Cas9 gene editing. The latter enabled high efficiency excision of large fragments of the viral genome from anatomically privileged tissues. Results support the idea that maximal viral restriction must be first established prior to excision to achieve optimal viral editing by CRISPR-Cas9.

Current HIV-1 treatment patterns are defined by daily dosing of a combination of either two nucleoside reverse transcriptase inhibitors (NRTIs) and one integrase strand transfer inhibitor (INSTI), or two NRTIs and one nonnucleoside reverse tran-scriptase inhibitor. Rebound that follows affects both the number and function of CD4⁺ T cells leading to virus-associated co-morbid conditions. LASER ART was developed in an attempt to eliminate these limitations and was shown effective in establishing drug depots in macrophages with sustained antiretroviral activities and reductions in HIV-1 proviral load beyond ART alone (Wainberg M A., et al., Can. J. Microbiol. 2016; 62:375-382. Landovitz R J, et al. Curr. Opin. HIV AIDS. 2016; 11:122-128. Larraneta E, et al. Pharm. Res. 2016; 33:1055-1073. Gunawardana M, et al. Antimicrob. Agents Chemother. 2015; 59:3913-3919) The success in these prior studies led to the use of LASER ART in the current report in order to maximize ART ingress to cell and tissue sites of viral replication enabling the drugs to reach these sites at high concentrations for sustained time periods. The maintenance of slow drug release for times measured in weeks or longer provided optimal settings for viral excision (Martinez-Skinner A L, et al. PLoS ONE. 2015; 10:e0145966. Doshi N, Mitragotri S. PLoS ONE. 2010;5:e10051. Lepik K J, et al. AIDS. 2017;31:1425-1434). ART particles coated with poloxamers enabled lipophilic hydrophobic prodrug crystals to readily cross cell and tissue barriers, aiding precision drug release to viral sanctuary sites. These claims are reinforced by the prior studies demonstrating up to a 10-fold increase in viral restriction at two independent multiplicities of infection in CD4⁺ T cell lines with LASER ART when compared to conventional native drugs (Guo D, et al. J. Acquir. Immune Defic. Syndr. 2017; 74:e75-e83. Singh D, et al. Nanomedicine. 2016; 11:1913-1927). The advantages of LASER ART over native ART include rapid entry across cell membranes of both CD4⁺ T cells and macrophages (due to drug lipophilicity); accelerated antiretroviral drug entry into viral reservoir sites (including the brain, gut, lymph nodes, liver, bone marrow and spleen); increased intracellular drug delivery; and stable plasma concentrations observed over weeks to months. The ART were selected in order to produce sustained plasma concentrations 4× the protein-adjusted 90% inhibitory concentration. Notably, a single parenteral dose of NMDTG at 45 mg DTG equivalents/kg to mice provided plasma DTG concentration of 88 ng/ml at 56 days³². Liver, spleen and lymph node DTG concentrations were 8.0, 31.2 and 17.6 ng/g, respectively at 56 days following single treatment. At 14 days after NMABC and NM3TC given at 50 mg ABC or 3TC equivalents/kg to mice, ABC and 3TC plasma concentrations were 21 and <7 ng/ml, respectively. In summary, there was little to no residual ART in plasma or tissue at the time of animal sacrifice reflecting the robust viral rebound found in all infected mice treated with LASER ART alone. Further, significant efforts were made by us to demonstrate that one month after LASER ART was discontinued, viral rebound was detectable. All of this highlights the rationale for use of LASER ART over native ART. ART levels in plasma were undetectable during the period of measured viral rebound.

For elimination of proviral DNA, the CRISPR-Cas9 gene editing platform was chosen and a multiplex of gRNAs were created that caused cleavage of the viral genome at the highly conserved regions within the LTRs and the Gag gene. This strategy allowed for the removal of the large intervening DNA fragments across the viral genome and mitigated any chance for the emergence of virus escape mutants. In support of this notion, results from cell culture and animal adoptive infection studies showed the absence of replication competent HIV-1 in the spleen and bone marrow of animals with no rebound that could be attributed to virus escape. The choice for the use of AAV9 comes from earlier studies demonstrating the broad range tissue distribution of CRISPR-Cas9 in a mouse model (Pino S, et al. Methods Mol. Biol. 2010; 602:105-117). Accordingly, the results in this current study verified the bioavailability of the gene editing molecule in various organs of the NSG humanized mice. No off-target effects were detected in in vivo deep sequencing and bioinformatics analysis that may be caused by the CRISPR-Cas9 editing strategy. Nevertheless, naturally occurring cellular DNA variation was found in both untreated cells as well as in CRISPR-Cas9-treated cells. Examination of several potential target cellular genes performed on clonal cells expressing CRISPR-Cas9 by gene amplification and direct sequencing showed no mutations that may be caused by the presence of CRISPR-Cas9 in the cells.

Results from ddPCR showed 60% to 80% efficiency of viral DNA excision by CRISPR-Cas9. Of note, this approach quantified dual cleavage events that removed the DNA fragment spanning 5′LTR to 3′LTR, 5′LTR to gag, and gag to 3′LTR of the proviral genome. However, the occurrence of single site editing events that would permanently interrupt the viral DNA and potentially inactivate viral replication by introducing small InDel mutations at the cleavage sites are not included in this estimate. Therefore, viral activation and rebound may not be observed under the conditions whereby excision efficiency is less than 100%. Inclusion of quadruplex of gRNAs for targeting Gag, Pol and two separate sites within the LTRs may yield slightly higher efficiency of viral DNA excision. In recent studies, bioimaging, antiretroviral PK and sensitive tissue biodistribution studies were combined to facilitate ART delivery into cell and tissue viral reservoirs in both humanized mice and non-human primates. These combined diagnostic and therapeutic modalities, coined theranostics, are being developed to facilitate effective HIV-1 elimination strategies in an infected human host (Williams J., et al. Nanomedicine. 2013;8:1807-1813).

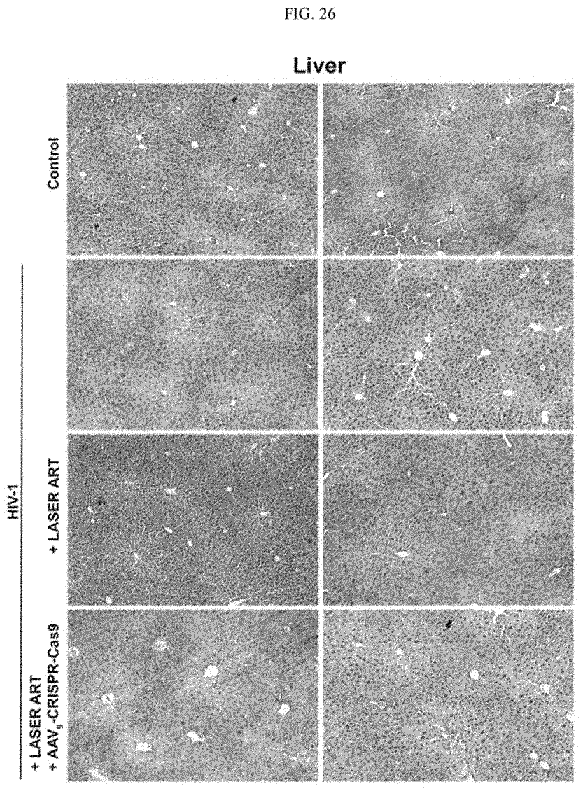

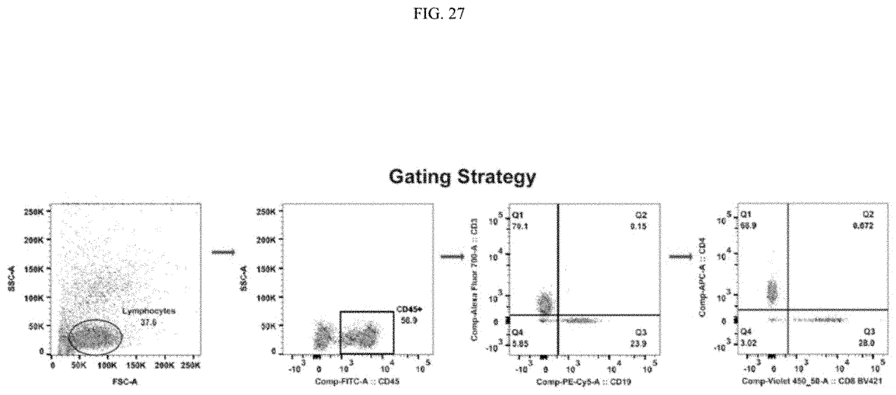

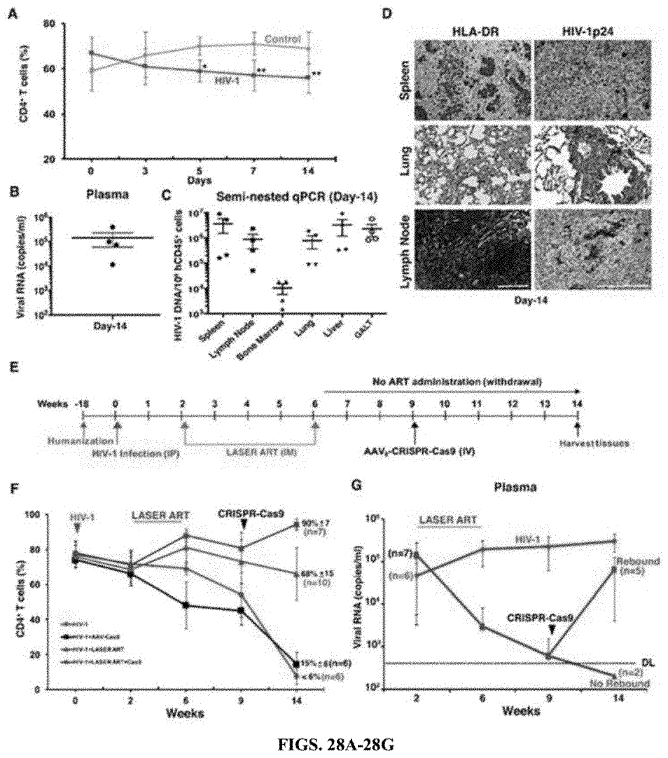

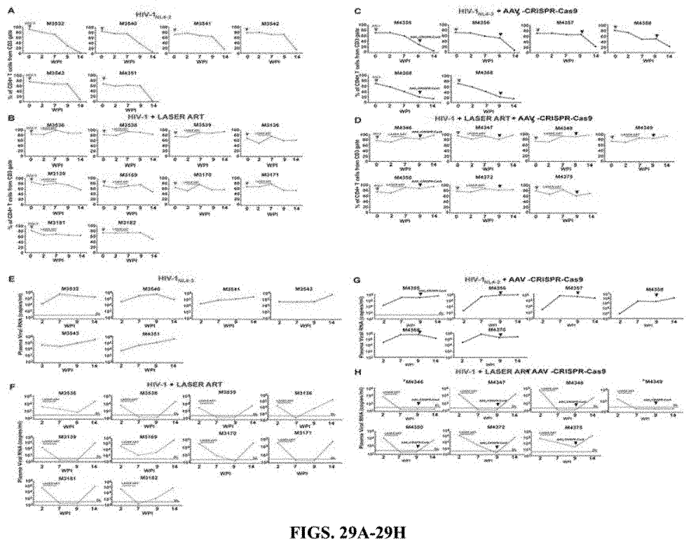

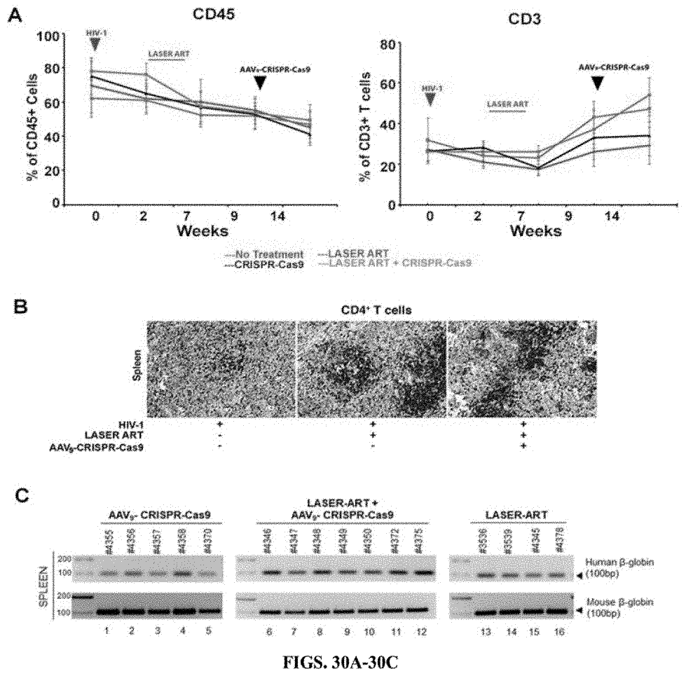

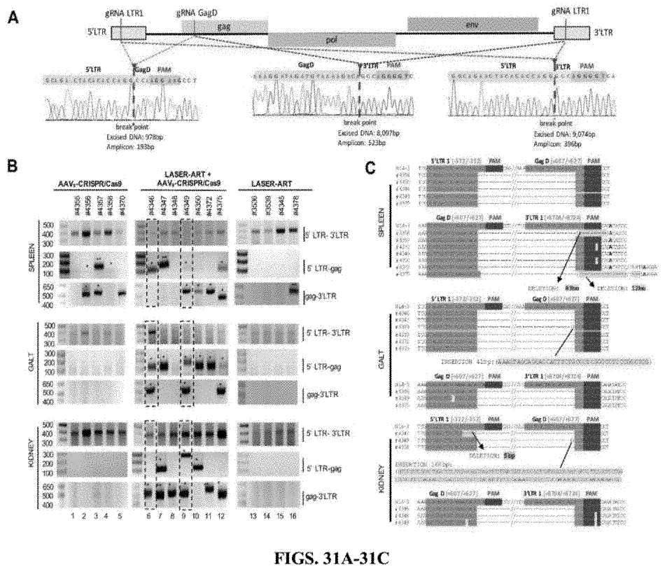

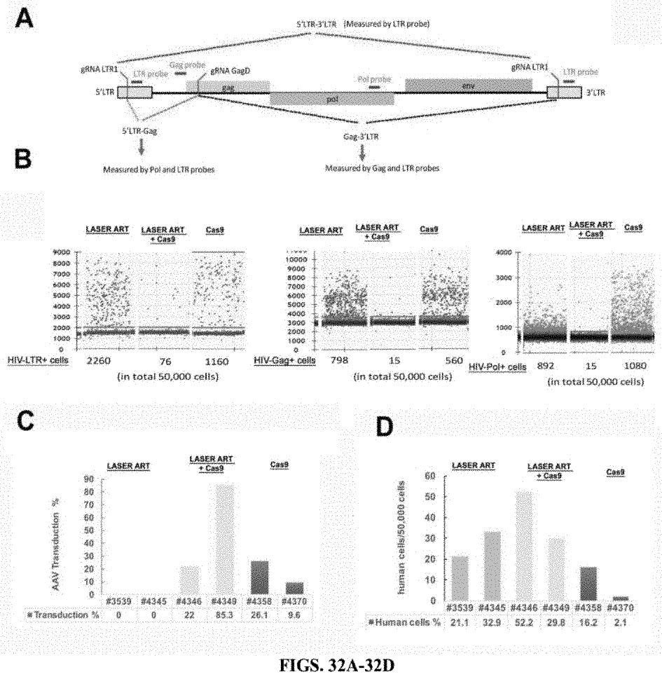

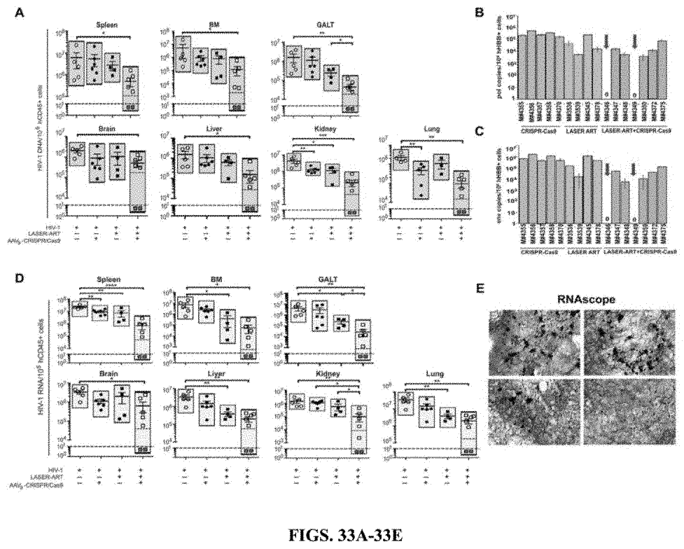

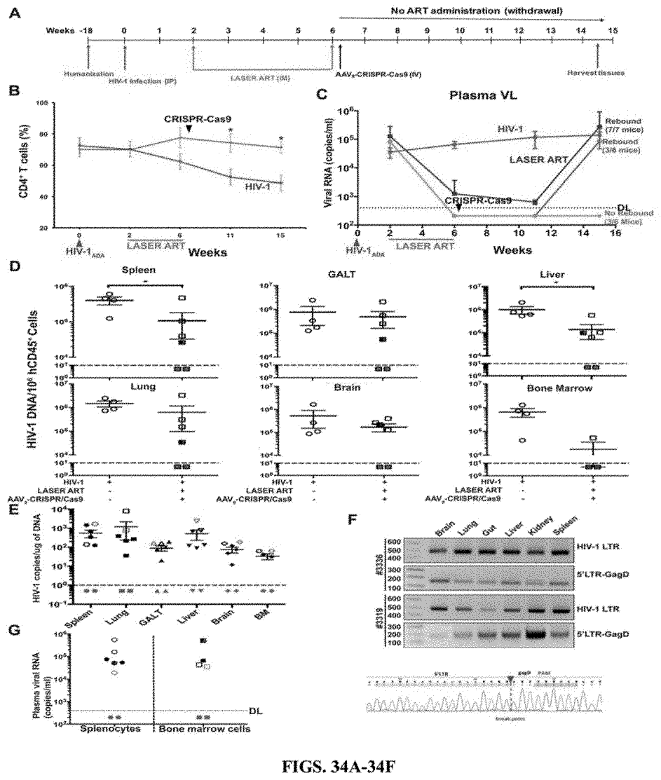

In conclusion, a broad range of highly sensitive tests to evaluate HIV-1 elimination by LASER ART and AAV9-delivered CRISPR-Cas9 treatments was employed. These included viral gene amplification, flow cytometry, adoptive viral transfers, on target and off target assays, and measures of viral rebound to demonstrate that combination therapies can safely lead to the elimination of HIV-1 infection. Results demonstrated that eradication of replication-competent HIV-1 present in infectious cell and tissue sites of infected animals can be achieved. Although reappearance of viremia in humans can be delayed, rebound occurs on average 2 to 4 weeks after ART interruption and 5 to 11 days in humanized mice. Despite the vigorous treatments offered, there was no evidence of outward untoward effects of any therapies (FIG. 26) including the persistence of human adult lymphocytes in mouse plasma and tissue. As such, these results offer readily defined and realistic pathways toward strategies for HIV-1 elimination.

### REFERENCES

1. G. Huffer et al., Long-term control of HIV by CCR5 Delta32/Delta32 stem-cell transplantation. The New England journal of medicine 360, 692-698 (2009).
2. W. Xu et al., Advancements in Developing Strategies for Sterilizing and Functional HIV Cures. BioMed research international 2017, 6096134 (2017).
3. A. Saez-Cirion et al., Post-treatment HIV-1 controllers with a long-term virological remission after the interruption of early initiated antiretroviral therapy ANRS VISCONTI Study. PLoS pathogens 9, e1003211 (2013).
4. J. Z. Li et al., The size of the expressed HIV reservoir predicts timing of viral rebound after treatment interruption. AIDS (London, England) 30, 343-353 (2016).
5. J. D. Siliciano, R. F. Siliciano, Recent developments in the effort to cure HIV infection: going beyond N=1. The Journal of clinical investigation 126, 409-414 (2016).
6. A. R. Martin, R. F. Siliciano, Progress Toward HIV Eradication: Case Reports, Current Efforts, and the Challenges Associated with Cure. Annual review of medicine 67, 215-228 (2016).
   D. Singh et al., Development and characterization of a long-acting nanoformulated abacavir prodrug. Nanomedicine (London, England) 11, 1913-1927 (2016).
8. D. Guo et al., Creation of a Long-Acting Nanoformulated 2′,3′-Dideoxy-3′-Thiacytidine. Journal of acquired immune deficiency syndromes (1999) 74, e75-e83 (2017).
9. B. Edagwa, et al, Long-acting slow effective release antiretroviral therapy. Expert opinion on drug delivery, 1-11 (2017).
10. P. K. Dash et al., Long-acting nanoformulated antiretroviral therapy elicits potent antiretroviral and neuroprotective responses in HIV-1-infected humanized mice. AIDS (London, England) 26, 2135-2144 (2012).
11. Hu et. al, RNA-directed gene editing specifically eradicates latent and prevents new HIV-1 infection. Proc.Natl.Acad.Sci. USA 111,11461-11466 (2014)
12. R. Kaminski et al., Elimination of HIV-1 Genomes from Human T-lymphoid Cells by CRISPR/Cas9 Gene Editing. Scientific reports 6, 22555 (2016).
13. R. Kaminski et al., Negative Feedback Regulation of HIV-1 by Gene Editing Strategy. Scientific reports 6, 31527 (2016).
14. M. K. White, W. Hu, K. Khalili, Gene Editing Approaches against Viral Infections and Strategy to Prevent Occurrence of Viral Escape. PLoS pathogens 12, e1005953 (2016).
15. R. Kaminski et al., Excision of HIV-1 DNA by gene editing: a proof-of-concept in vivo study. Gene therapy 23, 690-695 (2016).
16. C. Yin et al., In Vivo Excision of HIV-1 Provirus by saCas9 and Multiplex Single-Guide RNAs in Animal Models. Molecular therapy: the journal of the American Society of Gene Therapy 25, 1168-1186 (2017).
17. M. Arainga, et al, HIV-1 cellular and tissue replication patterns in infected humanized mice. Scientific reports 6, 23513 (2016).
18. M. Arainga et al., A mature macrophage is a principal HIV-1 cellular reservoir in humanized mice after treatment with long acting antiretroviral therapy. Retrovirology 14, 17 (2017).
19. R. C. Gallo, Shock and kill with caution. Science (New York, N.Y.) 354, 177-178 (2016).
20. S. N. Byrareddy et al., Sustained virologic control in SIV+macaques after antiretroviral and alpha4beta7 antibody therapy. Science (New York, N.Y.) 354, 197-202 (2016).
21. S. Gorantla et al., Human immunodeficiency virus type 1 pathobiology studied in humanized BALB/c-Rag2−/−gammac−/− mice. Journal of virology 81, 2700-2712 (2007).
22. S. Gorantla et al., Links between progressive HIV-1 infection of humanized mice and viral neuropathogenesis. Am J Pathol 177, 2938-2949 (2010).
23. J. M. Jacobson et al., Evidence that intermittent structured interruption, but not immunization with ALVAC-HIV vCP 1452, promotes control of HIV replication: the results of AIDS Clinical Trials Group. J. Infect Dis. 194, 623-632 (2006).
24. J. B. Honeycutt et al., HIH persistence in tissue macrophages of humanized myeloid-only mice during antiretroviral therapy. Nat. Med. 23, 638-643 (2017).
25. R. H. Kutner et al., Production, concentration and titration of pseudotyped HIV-1-based lentiviral vectors. Nature Protocols 4, 495-505 (2009).
26. O'Doherty U et al., Human Immunodeficiency Virus Type 1 spinoculation enhances infection through virus binding. Journal of Virology 74(21), 10074-10080 (2000).
27. P. K. Dash et al., Loss of neuronal integrity during progressive HIV-1 infection of humanized mice. J Neurosci 31, 3148-3157 (2011).
28. Westervelt, et al, Identification of a determinant within the human immunodeficiency virus 1 surface envelope glycoprotein critical for productive infection of primary monocytes. Proceedings of the National Academy of Sciences of the United States of America 88, 3097-3101 (1991).
29. G. Zhang et al., The mixed lineage kinase-3 inhibitor URMC-099 improves therapeutic outcomes for long-acting antiretroviral therapy. Nanomedicine: nanotechnology, biology, and medicine 12, 109-122 (2016).
30. A. S. Nowacek et al., NanoART synthesis, characterization, uptake, release and toxicology for human monocyte-macrophage drug delivery. Nanomedicine (London, England) 4, 903-917 (2009).
31. L. M. Agosto et al., HIV-1 integrates into resting CD4+ T cells even at low inoculums as demonstrated with an improved assay for HIV-1 integration. Virology 368, 60-72 (2007).
32. A. O. Pasternak et al., Highly sensitive methods based on seminested real-time reverse transcription-PCR for quantitation of human immunodeficiency virus type 1 unspliced and multiply spliced RNA and proviral DNA. Journal of clinical microbiology 46, 2206-2211 (2008).
33. M. K. Liszewski, et al, Detecting HIV-1 integration by repetitive-sampling Alu-gag PCR. Methods (San Diego, Calif.) 47, 254-260 (2009).
34. C. Deleage et al., Defining HIV and SIV Reservoirs in Lymphoid Tissues. Pathogens &
35. M. J. Buzon et al., Long-term antiretroviral treatment initiated at primary HIV-1 infection affects the size, composition, and decay kinetics of the reservoir of HIV-1-infected CD4 T cells. Journal of virology 88, 10056-10065 (2014).
36. M. J. Buzon et al., HIV-1 persistence in CD4+ T cells with stem cell-like properties. Nature medicine 20, 139-142 (2014).
37. G. M. Laird et al., Rapid quantification of the latent reservoir for HIV-1 using a viral outgrowth assay. PLoS pathogens 9, e1003398 (2013).

### Example 2

### Combination of CRISPR and LASER ART Eliminates Replication Competent Rebound in Humanized Mice

Advances in CRISPR-Cas9gene editing technology and its in vivo delivery by AAV9 vectors together with cell based nanotechnology for long-acting slow effective release antiretroviral therapy (LASER-ART), were used in NSG-CD34 humanized mice to facilitate eradication of HIV-1 in vivo.

Methods

CRISPR-Cas9 proviral DNA excision followed two months of treatment with long-acting slow effective release antiretroviral therapy (LASER-ART), rilpivirine, myristolyated dolutegravir, lamivudine, and abacavir in HIV-1 infected humanized mice. A series of virological, histological, and DNA and RNA assays were used to detect HIV-1 expression and replication in the animal tissues. Ultra deep, whole genome sequencing was employed to assess in vivo off-target effects.

Results

Results from three independent sets of studies showed restorations of CD4⁺ T cells due to ART treatment and complete eradication of replication competent virus by CRISPR in 39% of animals. Ultrasensitive nested and digital droplet PCR and RNA scope assays failed to detect HIV-1 in blood, spleen, lung, kidney, liver, gut-associated lymphoid tissue and brain. Excision of proviral DNA fragments spanning the LTRs and the Gag gene from the integrated proviral DNA was identified, while no off target effects were observed. The absence of viral rebound following cessation of ART with no progeny virus recovery after in vivo adoptive transfer of human immunocytes from dual-treated virus-free animals to uninfected humanized mice verified HIV-1 eradication by the combined treatment strategy. In contrast, HIV-1 was readily detected in all infected animals treated with LASER ART or CRISPR-Cas9 alone.

Conclusions

The sequential application of LASER ART and CRISPR-Cas9 therapies administered to HIV-1 infected humanized mice provides the first proof-of-concept that viral sterilization is possible.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

What is claimed:

1. A method of eradicating or eliminating a retrovirus in a subject, comprising administering to a patient a composition comprising a therapeutically effective amount of at least one antiretroviral agent and/or a composition comprising a therapeutically effective amount of at least one gene editing agent, thereby eradicating or eliminating the retrovirus in a subject.

2. The method of claim 1, wherein the antiretroviral agent is formulated as a long-acting slow effective release (LASER) antiretroviral agent.

3. The method of claim 2, wherein the at least one antiretroviral agent is nanoformulated.

4. The method of claim 3, wherein the at least one antiretroviral agent comprises: myristolyated dolutegravir, lamivudine, abacavir, rilpivirine or combinations thereof.

5. The method of claim 1, wherein the at least one antiretroviral agent is administered to the subject prior to administering the at least one gene editing agent.

6. The method of claim 1, wherein the at least one antiretroviral agent and at least one gene-editing agent are co-administered.

7. The method of claim 1, wherein the at least one antiretroviral agent and at least one gene-editing agent are administered sequentially.

8. The method of claim 1, wherein the at least one gene editing agent comprises: an isolated nucleic acid sequence encoding a Clustered Regularly Interspaced Short Palindromic Repeat (CRISPR)-associated endonuclease/Cas (CRISPR/Cas) and at least one guide RNA (gRNA), the gRNA being complementary to a target nucleic acid sequence in a retroviral genome.

9. The method of claim 8, wherein the CRISPR/Cas fusion protein comprises catalytically deficient Cas protein (dCas), orthologs, homologs, mutants variants or fragments thereof.

10. The method of claim 8, wherein the at least one gRNA includes at least a first gRNA that is complementary to a target sequence in the integrated retroviral DNA; and a second gRNA that is complementary to another target sequence in the integrated retroviral DNA, whereby the intervening sequences between the two gRNAs are removed.

11. The method of claim 8, wherein the isolated nucleic acid is included in at least one expression vector.

12. The method of claim of claim 11, wherein the expression vector comprises a lentiviral vector, an adenoviral vector, or an adeno-associated virus vector.

13. The method of claim 8, wherein the retrovirus is a human immunodeficiency virus (HIV).

14. The method of claim 13, wherein the target sequences comprise one or more nucleic acid sequences in HIV comprising: long terminal repeat (LTR) nucleic acid sequences, nucleic acid sequences encoding structural proteins, non-structural proteins or combinations thereof.

15. The method of claim 14, wherein the sequences encoding structural proteins comprise nucleic acid sequences encoding: Gag, Gag-Pol precursor, Pro (protease), Reverse Transcriptase (RT), integrase (In), Env or combinations thereof.

16. The method of claim 14, wherein the sequences encoding non-structural proteins comprise nucleic acid sequences encoding: regulatory proteins, accessory proteins or combinations thereof.

17. The method of claim 16, wherein regulatory proteins comprise: Tat, Rev or combinations thereof.

18. The method of claim 16, wherein accessory proteins comprise Nef, Vpr, Vpu, Vif or combinations thereof.

19. The method of claim 1, wherein a gRNA comprises at least one nucleic acid sequence set forth in Tables 1-5 or combinations of gRNAs.

20. The method of claim 1, optionally comprising a therapeutically effective amount of a non-nucleoside reverse transcriptase inhibitor (NNRTI), and/or a nucleoside reverse transcriptase inhibitor (NRTI) and/or a protease inhibitor.

21. The method of claim 20, wherein the NNRTI comprises: etravirine, efavirenz, nevirapine, rilpivirine, delavirdine, or nevirapine.

22. The method of claim 20, wherein the NRTI comprises lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof.

23. The method of claim 20, wherein a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

24. A pharmaceutical composition comprising a therapeutically effective amount of a nanoformulated long-acting slow effective release antiretroviral agent.

25. The pharmaceutical composition of claim 24, wherein the nanoformulated antiretroviral agent comprises: myristolyated dolutegravir, lamivudine, abacavir, rilpivirine or combinations thereof.

26. The pharmaceutical composition of claim 24, further comprising at least one an isolated nucleic acid sequence encoding a Clustered Regularly Interspaced Short Palindromic Repeat (CRISPR)-associated endonuclease; at least one isolated nucleic acid sequence encoding at least one guide RNA (gRNA) that is complementary to a target sequence in retroviral DNA; said isolated nucleic acid sequences being included in at least one expression vector.

27. The pharmaceutical composition of claim 26, wherein the integrated retroviral DNA is human immunodeficiency virus (HIV) DNA, and said at least one gRNA includes a first gRNA that is complementary to a first target sequence in the HIV DNA, and a second gRNA that is complementary to a second target sequence in the HIV DNA.
